SEQUENCE LISTING (99 sequences)

SEQ ID NO: 1 - RNA, 22 nt, Homo sapiens
gugaaauguuuaggaccacuag

SEQ ID NO: 2 - RNA, 22 nt, Homo sapiens
uagcuuaucagacugauguuga

SEQ ID NO: 3 - RNA, 21 nt, Homo sapiens
ggcaagaugcuggcauagcug

SEQ ID NO: 4 - RNA, 22 nt, Homo sapiens
uagcagcacguaaauauuggcg

SEQ ID NO: 5 - RNA, 22 nt, Homo sapiens
uagcagcacaucaugguuuaca

SEQ ID NO: 6 - RNA, 23 nt, Homo sapiens
ucccugagacccuuuaaccugug

SEQ ID NO: 7 - RNA, 22 nt, Homo sapiens
ucccugagacccuaacuuguga

SEQ ID NO: 8 - RNA, 22 nt, Homo sapiens
uaauacugccggguaaugaugg

SEQ ID NO: 9 - RNA, 21 nt, Homo sapiens
uggaauguaaagaaguaugua

SEQ ID NO: 10 - RNA, 23 nt, Homo sapiens
uacccuguagauccgaauuugug

SEQ ID NO: 11 - RNA, 22 nt, Homo sapiens
uacccuguagaaccgaauuugu

SEQ ID NO: 12 - RNA, 23 nt, Homo sapiens
caaagugcucauagugcagguag

SEQ ID NO: 13 - RNA, 21 nt, Homo sapiens
aucacauugccagggauuucc

SEQ ID NO: 14 - RNA, 21 nt, Homo sapiens
aucacauugccagggauuacc

SEQ ID NO: 15 - RNA, 23 nt, Homo sapiens
gugccuacugagcugauaucagu

SEQ ID NO: 16 - RNA, 22 nt, Homo sapiens
uucaaguaauccaggauaggcu

SEQ ID NO: 17 - RNA, 21 nt, Homo sapiens
uucaaguaauucaggauaggu

SEQ ID NO: 18 - RNA, 21 nt, Homo sapiens
uucacaguggcuaaguuccgc

SEQ ID NO: 19 - RNA, 22 nt, Homo sapiens
aaggagcucacagucuauugag

SEQ ID NO: 20 - RNA, 21 nt, Homo sapiens
uagcaccaucugaaaucgguu

SEQ ID NO: 21 - RNA, 22 nt, Homo sapiens
uagcaccauuugaaaucgguua

SEQ ID NO: 22 - RNA, 22 nt, Homo sapiens
uguaaacauccucgacuggaag

SEQ ID NO: 23 - RNA, 22 nt, Homo sapiens
cuuucagucggauguuugcagc

SEQ ID NO: 24 - RNA, 22 nt, Homo sapiens
uguaaacauccuacacucagcu

SEQ ID NO: 25 - RNA, 23 nt, Homo sapiens
uguaaacauccuacacucucagc

SEQ ID NO: 26 - RNA, 20 nt, Homo sapiens
uguaaacauccuugacugga

SEQ ID NO: 27 - RNA, 22 nt, Homo sapiens
cuuucagucggauguuuacagc

SEQ ID NO: 28 - RNA, 22 nt, Homo sapiens
uucaacggguauuuauugagca

SEQ ID NO: 29 - RNA, 22 nt, Homo sapiens
aacccguagauccgaucuugug

SEQ ID NO: 30 - RNA, 22 nt, Homo sapiens
cacccguagaaccgaccuugcg

SEQ ID NO: 31 - RNA, 22 nt, Homo sapiens
aacccguagauccgaacuugug

SEQ ID NO: 32 - RNA, 22 nt, Homo sapiens
uacaguacugugauaacugaag

SEQ ID NO: 33 - RNA, 22 nt, Homo sapiens
ucguaccgugaguaauaaugcg

SEQ ID NO: 34 - RNA, 22 nt, Homo sapiens
ucggauccgucugagcuuggcu

SEQ ID NO: 35 - RNA, 22 nt, Homo sapiens
cagugcaauguuaaaagggcau

SEQ ID NO: 36 - RNA, 21 nt, Homo sapiens
uugguccccuucaaccagcua

SEQ ID NO: 37 - RNA, 23 nt, Homo sapiens
uauggcuuuucauuccuauguga

SEQ ID NO: 38 - RNA, 18 nt, Homo sapiens
ucuacagugcacgugucu

SEQ ID NO: 39 - RNA, 23 nt, Homo sapiens
guccaguuuucccaggaaucccu

SEQ ID NO: 40 - RNA, 21 nt, Homo sapiens
uugguccccuucaaccagcua

SEQ ID NO: 41 - RNA, 21 nt, Homo sapiens
agugguuuuacccuaugguag

SEQ ID NO: 42 - RNA, 22 nt, Homo sapiens
ugagaugaagcacuguagcuca

SEQ ID NO: 43 - RNA, 22 nt, Homo sapiens
ucagugcacuacagaacuuugu

SEQ ID NO: 44 - RNA, 22 nt, Homo sapiens
ucagugcaucacagaacuuugu

SEQ ID NO: 45 - RNA, 23 nt, Homo sapiens
ucuggcuccgugucuucacuccc

SEQ ID NO: 46 - RNA, 21 nt, Homo sapiens
ucagugcaugacagaacuugg

SEQ ID NO: 47 - RNA, 23 nt, Homo sapiens
aacauucauuguugucggugggu

SEQ ID NO: 48 - RNA, 24 nt, Homo sapiens
uuuggcaaugguagaacucacacu

SEQ ID NO: 49 - RNA, 23 nt, Homo sapiens
caacggaaucccaaaagcagcug

SEQ ID NO: 50 - RNA, 21 nt, Homo sapiens
aacuggccuacaaagucccag

SEQ ID NO: 51 - RNA, 22 nt, Homo sapiens
aacuggcccucaaagucccgcu

SEQ ID NO: 52 - RNA, 21 nt, Homo sapiens
uagcagcacagaaauauuggc

SEQ ID NO: 53 - RNA, 21 nt, Homo sapiens
uagguaguuucauguuguugg

SEQ ID NO: 54 - RNA, 22 nt, Homo sapiens
uagguaguuuccuguuguuggg

SEQ ID NO: 55 - RNA, 22 nt, Homo sapiens
uucaccaccuucuccacccagc

SEQ ID NO: 56 - RNA, 23 nt, Homo sapiens
cccaguguucagacuaccuguuc

SEQ ID NO: 57 - RNA, 23 nt, Homo sapiens
cccaguguuuagacuaucuguuc

SEQ ID NO: 58 - RNA, 22 nt, Homo sapiens
uucccuuugucauccuaugccu

SEQ ID NO: 59 - RNA, 22 nt, Homo sapiens
cugugcgugugacagcggcuga

SEQ ID NO: 60 - RNA, 22 nt, Homo sapiens
uucccuuugucauccuucgccu

SEQ ID NO: 61 - RNA, 22 nt, Homo sapiens
acagcaggcacagacaggcagu

SEQ ID NO: 62 - RNA, 21 nt, Homo sapiens
uugugcuugaucuaaccaugu

SEQ ID NO: 63 - RNA, 23 nt, Homo sapiens
agcuacauugucugcuggguuuc

SEQ ID NO: 64 - RNA, 24 nt, Homo sapiens
agcuacaucuggcuacugggucuc

SEQ ID NO: 65 - RNA, 21 nt, Homo sapiens
ugucaguuugucaaauacccc

SEQ ID NO: 66 - RNA, 21 nt, Homo sapiens
caagucacuagugguuccguu

SEQ ID NO: 67 - RNA, 21 nt, Homo sapiens
agggcccccccucaauccugu

SEQ ID NO: 68 - RNA, 23 nt, Homo sapiens
cgcauccccuagggcauuggugu

SEQ ID NO: 69 - RNA, 20 nt, Homo sapiens
acugccccaggugcugcugg

SEQ ID NO: 70 - RNA, 22 nt, Homo sapiens
cuggcccucucugcccuuccgu

SEQ ID NO: 71 - RNA, 23 nt, Homo sapiens
ucaagagcaauaacgaaaaaugu

SEQ ID NO: 72 - RNA, 22 nt, Homo sapiens
gcugacuccuaguccagggcuc

SEQ ID NO: 73 - RNA, 22 nt, Homo sapiens
uaaugccccuaaaaauccuuau

SEQ ID NO: 74 - RNA, 22 nt, Homo sapiens
uuauaauacaaccugauaagug

SEQ ID NO: 75 - RNA, 22 nt, Homo sapiens
uuuguucguucggcucgcguga

SEQ ID NO: 76 - RNA, 22 nt, Homo sapiens
cuccugacuccagguccugugu

SEQ ID NO: 77 - RNA, 22 nt, Homo sapiens
gaaguuguucgugguggauucg

SEQ ID NO: 78 - RNA, 22 nt, Homo sapiens
agaucagaaggugauuguggcu

SEQ ID NO: 79 - RNA, 21 nt, Homo sapiens
uaguagaccguauagcguacg

SEQ ID NO: 80 - RNA, 22 nt, Homo sapiens
agcucggucugaggccccucag

SEQ ID NO: 81 - RNA, 22 nt, Homo sapiens
cagcagcaauucauguuuugaa

SEQ ID NO: 82 - RNA, 23 nt, Homo sapiens
aaugacacgaucacucccguuga

SEQ ID NO: 83 - RNA, 23 nt, Homo sapiens
aaaccguuaccauuacugaguuu

SEQ ID NO: 84 - RNA, 22 nt, Homo sapiens
aacuguuugcagaggaaacuga

SEQ ID NO: 85 - RNA, 22 nt, Homo sapiens
uccuguacugagcugccccgag

SEQ ID NO: 86 - RNA, 22 nt, Homo sapiens
aaucguacagggucauccacuu

SEQ ID NO: 87 - RNA, 21 nt, Homo sapiens
cagcagcacacugugguuugu

SEQ ID NO: 88 - RNA, 23 nt, Homo sapiens
ugauugguacgucuguggguaga

SEQ ID NO: 89 - RNA, 20 nt, Homo sapiens
auugacacuucugugaguag

SEQ ID NO: 90 - RNA, 22 nt, Homo sapiens
caugccuugaguguaggaccgu

SEQ ID NO: 91 - RNA, 21 nt, Homo sapiens
uccgagccugggucucccucu

SEQ ID NO: 92 - RNA, 22 nt, Homo sapiens
uacccauugcauaucggaguug

SEQ ID NO: 93 - RNA, 22 nt, Homo sapiens
ugagguaguagguuguauaguu

SEQ ID NO: 94 - RNA, 22 nt, Homo sapiens
ugagguaguagguugugugguu

SEQ ID NO: 95 - RNA, 22 nt, Homo sapiens
ugagguaguagguuguaugguu

SEQ ID NO: 96 - RNA, 21 nt, Homo sapiens
agagguaguagguugcauagu

SEQ ID NO: 97 - RNA, 21 nt, Homo sapiens
ugagguaggagguuguauagu

SEQ ID NO: 98 - RNA, 22 nt, Homo sapiens
ugagguaguagauuguauaguu

SEQ ID NO: 99 - RNA, 22 nt, Homo sapiens
ugagguaguaguuuguacaguu

(19) United States
(12) Patent Application Publication
(10) Pub. No.: US 2011/0107440 A1
(43) Pub. Date: May 5, 2011

(54) SKIN CANCER ASSOCIATED MICRORNAS

(76) Inventors: Andor Pivarcsi, Stockholm (SE); Eniko Sonkoly, Stockholm (SE)

(21) Appl. No.: 12/994,734

(22) PCT Filed: Jun. 4, 2009
(86) PCT No.: PCT/IB2009/005946

§ 371 Date: Nov. 24, 2010

Related U.S. Application Data
(60) Provisional application No. 61/130,970, filed on Jun. 4, 2008.

(51) Int. Cl. A61K 49/00; C12Q 1/68; A61K 31/7088; C07H 21/00; C40B 30/04; A61P 35/00

(52) U.S. Cl. 800/3; 435/6; 514/44 R; 514/44 A; 536/24.5; 506/9

(57) ABSTRACT

This invention relates to the finding that skin cancers, such as squamous cell carcinoma and basal cell carcinoma, are characterized by changes in the expression of specific microRNA molecules (miRNAs). These miRNAs may therefore be useful as biomarkers for skin cancers as well as therapeutic targets. Methods of diagnosis and treatment of skin cancers are provided, as well as methods of screening for therapeutic compounds.

This invention relates to microRNA molecules (miRNAs) which are associated with non-melanoma skin cancers, such as squamous cell carcinoma and basal cell carcinoma.

Basal cell and squamous cell carcinoma (BCC and SCC) of the skin represent the most common malignancies in the Caucasian population with a total of 1.3 million new cases in the year 2000 in the United States alone, posing a significant threat to public health (American Cancer Society 2000). In men, they are more frequent than prostate carcinoma, and in women, they outnumber breast carcinoma (Urosevic and Dummer 2002). While BCC has no known precursor lesions, SCC presents a progressive state of a pre-cancerous lesion called actinic keratosis (AK) (Pivarcsi et al. 2007). The total ambulatory care costs for AK, SCC and BCC combined exceeds $3 billion/year.

The incidence of non-melanoma skin cancers, including metastatic SCC, is increasing due to the aging of the western society and because of its enormously increased incidence among organ transplant recipients. The incidence of SCC in transplant recipients is 40 to 250 times that of the general population, whereas the incidence of BCC is 10 times greater in transplant patients. SCCs in transplant patients are much more aggressive and deadly and out of the 5.1% of transplant patients who die from skin cancer, 60% had SCC and 33% had melanoma, which represents a 10-fold increase in mortality from SCC in comparison with the general population.

Due to the increasing prevalence of SCC in the Caucasian population, its mortality also shows an increasing trend, especially among immune suppressed individuals. As with other cancers, the main cause of mortality is metastasis formation, most frequently into the lungs or the lymph nodes.

The present inventors have discovered that non-melanoma skin cancer cells are characterized by specific, non-random microRNA (miRNA) expression profiles which differ from the miRNA expression profile of healthy skin. This indicates that miRNAs represent a previously unreported, epigenetic mechanism in skin cancer pathogenesis and may be useful in the diagnosis and therapy of non-melanoma skin cancers.

One aspect of the invention provides a method of assessing non-melanoma skin cancer in an individual comprising;
determining the expression of one or more of the miRNAs selected from the group consisting of miRNAs shown in Table 1 in a sample obtained from the individual.

A miRNA is a ribonucleic acid molecule of about 19 to 23 nucleotides, usually 21 to 22 nucleotides. miRNA molecules are naturally produced by higher eukaryotic cells and reduce the expression of specific protein-coding genes by targeting cognate messenger RNA for translational repression, mRNA destabilisation or a combination of the two. miRNAs are transcribed from non-protein-coding genes in the form of long primary transcripts (pri-miRNA). Pri-miRNAs are processed by a dsRNA-specific nuclease in the cell nucleus into hairpin RNA molecules of 70-100 nucleotides (pre-miRNA). These hairpin RNA molecules are further processed in the cytosol by a second dsRNA specific nuclease to produce the mature 19 to 23 nucleotide miRNA (Ambros, 2003; Bartel and Chen, 2004; Czech 2006).

The sequences of mature miRNAs described herein are set out in Table 16. The sequences of miRNA genes, precursors and mature miRNAs are also described in Lim L P, et al Science. 299:1540 (2003) and are publicly available from the miRNA Registry (miRBase) which is maintained by the Wellcome Trust Sanger Institute, Hinxton, UK. The miRBase database is described in Griffiths-Jones S, et al Nucleic Acids Res. 2008 36:D154-D158; Griffiths-Jones S, NAR, 2004, 32, D109-D111 and Griffiths-Jones S et al NAR, 2006, 34, D140-D144) and is available online at http://microrna.sanger.ac.uk/. To date, 678 human miRNAs have been registered in mirBase 13.0 (March 2009).

In the art, miRNAs are generally referred to by name. An assigned miRNA name refers unambiguously to a miRNA of a specific sequence. The annotation of miRNAs is described in Ambros V. et al RNA, 2003, 9(3), 277-279 and in the Sanger Institute's miRNA Registry database (http://microrna.sanger.ac.uk/sequences/).

The sample may be a sample of skin cells, serum or plasma.

Methods of assessing an individual as described herein may be useful for the diagnosis or prognosis of a non-melanoma skin cancer in an individual. For example, altered expression of the one or more miRNAs in the sample relative to controls may be indicative of the presence, type, tumour stage, severity, or risk of metastasis of a non-melanoma skin cancer in an individual.

Methods of assessing an individual as described herein may be useful in the assessing the susceptibility or risk of an individual suffering from a non-melanoma skin cancer. For example, altered expression of miRNAs as described herein relative to controls may be indicative that the individual is susceptible to or has a high risk of suffering from a skin cancer relative to control members of the population or may be indicative of the onset of the skin cancer.

Methods of assessing non-melanoma skin cancer in an individual as described herein may also be useful in determining the recurrence of a non-melanoma skin cancer in an individual following cancer therapy.

The expression of one or more miRNAs selected from the group consisting of the miRNAs listed in Table 2 may be determined. Increased expression of the one or more miRNAs in the sample relative to controls may be indicative of the presence, type, tumor stage, severity, or risk of metastasis of a non-melanoma skin cancer in an individual. For example, an increase in expression relative to controls may be indicative that the individual has a non-melanoma skin cancer, for example squamous cell carcinoma or basal cell carcinoma.

For example, the expression of miR-21 and/or miR-31 may be determined in the sample.

The expression of one or more miRNAs selected from the group consisting of the miRNAs listed in Table 3 may be determined in the sample. For example, the expression of one or more miRNAs selected from the group consisting of miR-203, miR-125b, miR-15b, miR-16, miR-193a and a let-7 miRNA, such as let-7g, may be determined in the sample. Decreased expression of the one or more miRNAs in the sample relative to controls may be indicative of the presence, type, tumor stage, severity, or risk of metastasis of a non-melanoma skin cancer in an individual. For example, a decrease relative to controls may be indicative that the individual has a non-melanoma skin cancer.

Non-melanoma skin cancers may include benign, pre-malignant and malignant tumours of keratinocytes, which are the predominant type of cutaneous epithelial cells. Keratinocyte cancers include epidermal tumours such as basal cell carcinoma (BCC), squamous cell carcinoma (SCC) or a pre-malignant lesion thereof, hair follicle tumors, such as trichoblastoma, trichoepitelioma, pilomatrixoma, pilomatrixcarcinoma, trichoadenoma, trichofolliculoma; sweat gland tumors such as adnexcarcinoma, mucinous eccrin carcinoma, porocarcinoma; and premalignant lesions of the skin such as actinic keratosis, morbus Bowen, and erythroplasia Queyrat.

In some embodiments, a change in the expression of one or more miRNAs is specifically indicative of Squamous Cell Carcinoma (SCC). SCC is an aggressive keratinocyte carcinoma which commonly metastasizes, following local invasion and tissue destruction. SCC is characterised by the presence of epidermal differentiation and the absence of a well-demarcated tumour periphery. SCC is associated with pre-cancerous lesions, such as actinic keratosis (AK) and Bowen's disease. In some embodiments, the term SCC may also encompass pre-cancerous lesions of SCC.

An increase in expression of one or more miRNAs set out herein may be indicative of the presence, type, tumor stage, severity, or risk of metastasis of SCC. A method of assessing SCC in an individual may comprise the step of determining the expression of one or more miRNAs selected from the group consisting of the miRNAs listed in Table 4 in a sample of skin cells, plasma or serum obtained from the individual. For example, the expression of miR-21 and/or miR-31 and, optionally, one or more additional miRNAs from Table 4a and/or 4b may be determined.

An increase in expression of the one or more miRNAs in the sample relative to controls may be indicative of the presence, type, tumor stage, severity, or risk of metastasis of a non-melanoma skin cancer in an individual. For example, an increased in the sample relative to controls may be indicative of the presence of SCC in the individual.

In some embodiments, the expression of one or more miRNAs selected from the group consisting of the miRNAs listed in Tables 12a and/or 12b may be determined. Preferably, the expression of miRNAs with high analysis scores is determined. These miRNAs are up-regulated in SCC relative to BCC. Determining the expression of one or more miRNAs listed in Tables 12a and/or 12b may be useful, for example, in distinguishing SCC from BCC in an individual. An increase in expression of the one or more miRNAs listed in Tables 12a and/or 12b in the sample relative to controls may be indicative that the individual has SCC.

A decrease in expression of one or more miRNAs set out herein may be indicative of the presence, type, tumor stage, severity, or risk of metastasis of SCC. For example, a method of assessing SCC in an individual may comprise determining the expression of one or more miRNAs selected from the group consisting of the miRNAs listed in Table 9a and/or Table 9b in a sample of skin cells, serum or plasma obtained from the individual.

In some preferred embodiments, the expression of one or more miRNAs selected from the group consisting of the miRNAs listed in Table 5 may be determined. For example, the expression of miR-125b, miR-15 and/or a let-7 miRNA, such as let-7g, and, optionally, one or more additional miRNAs from Table 5, may be determined in the sample.

A decrease in expression of the one or more miRNAs in the sample relative to controls is indicative of the presence, type, tumor stage, severity, or risk of metastasis of SCC. For example, decreased expression may be indicative that the individual has SCC.

The expression of one or more miRNAs selected from the group consisting of one or more miRNAs listed in Table 11 may be determined. These miRNAs are down-regulated in SCC relative to BCC. Determining the expression of these miRNAs may therefore be useful in distinguishing SCC from BCC in an individual. A decrease in expression of the one or more miRNAs listed in Table 11a and/or 11b in the sample relative to controls is indicative that the individual has SCC.

In other embodiments, a change in the expression of one or more miRNAs set out herein is specifically indicative of Basal Cell Carcinoma (BCC). BCC is a malignant keratinocyte tumour which is characterised by a well-demarcated tumour periphery and the absence of epidermal differentiation. BCC lacks pre-cancerous lesions. Although rarely metastatic, BCC may cause local tissue destruction.

An increase in expression of one or more miRNAs set out herein may be indicative of BCC. A method of assessing BCC in an individual may comprise;
determining the expression of one or more miRNAs selected from the group consisting of the miRNAs listed in Tables 6a and/or 6b.

Increased expression of the one or more miRNAs relative to controls may be indicative of the presence, type, tumor stage, severity, or risk of metastasis of BCC in the individual. For example, increased expression relative to controls may be indicative that the individual has BCC.

The expression of one or more miRNAs selected from the group consisting of one or more miRNAs listed in Table 11 may be determined. These miRNAs are up-regulated in BCC relative to SCC. Determining the expression of these miRNAs may therefore be useful in distinguishing BCC from SCC in an individual. An increase in expression of the one or more miRNAs listed in Tables 11a and/or 11b in the sample relative to controls is indicative that the individual has BCC.

A decrease in expression of one or more miRNAs set out herein may be indicative of BCC. A method of assessing BCC in an individual may comprise:
determining the expression of one or more miRNAs selected from the group consisting of the miRNAs listed in Table 10a and/or table 10b.

In some preferred embodiments, the expression of one or more miRNAs selected from the group consisting of the miRNAs listed in Table 7 may be determined. For example, the expression of miR-203, miR-15b, miR-16 and/or miR-193a may be determined, and, optionally, one or more additional miRNAs from Table 7.

A decrease in expression of the one or more miRNAs in the sample cells relative to controls is indicative of the presence, type, tumor stage, severity, or risk of metastasis of BCC in the individual. For example, decreased expression may be indicative that the individual has BCC.

The expression of one or more miRNAs selected from the group consisting of one or more miRNAs listed in Table 12 may be determined. These miRNAs are down-regulated in BCC relative to SCC. Determining the expression of these miRNAs may therefore be useful in distinguishing BCC from SCC in an individual. A decrease in expression of the one or more miRNAs listed in Tables 12a and/or 12b in the sample relative to controls may be indicative that the individual has BCC.

The methods described above may comprise determining the expression of one or more, two or more, three or more, four or more, five or more, six or more, seven or more, eight or more, nine or more, ten or more, fifteen or more or twenty or more of the listed miRNAs.

The data herein shows the analysis scores for each miRNA which are indicative of the degree of association of the miRNA with non-melanoma skin cancer. Preferably, the expression of miRNAs with the highest analysis scores is determined. For example, a method may comprise determining the expression of the miRNAs shown in the appropriate table below with the 1, 2, 3, 4, 5, 6, 7, 8, 9 or 10 highest analysis scores. A method may comprise determining the expression of 1, 2, 3, 4, 5, 6, 7, 8, 9 or 10 miRNAs selected from the group consisting of the miRNAs in the appropriate table below with the ten highest analysis scores. Optionally, the expression one or more additional miRNAs listed in the Table may be determined.

Certain miRNAs described herein are members of closely related families of miRNAs. miRNA families are groupings of miRNAs that share a common conserved seed region spanning nucleotides 2-7 (Lewis et al. Cell 2005, 120 15-20). The expression of one member of family of miRNAs may be indicative of the expression of other members of the same miRNA family. An increase or decrease in expression of one member of family of miRNAs may therefore be indicative that the expression of other members of the same miRNA family is also increased or decreased.

In some embodiments, a method described herein may comprise determining the expression of a first member of a miRNA family and inferring the expression of other members of the family from the amount of expression determined.

miRNA families include the let-7, miR-30, miR-125, miR-10, and miR-99 miRNA families.

The Let-7 miRNA family includes hsa-let-7a, hsa-let-7b, hsa-let-7c, hsa-let-7d, hsa-let-7e, hsa-let-7f, hsa-let-7g, hsa-let-71 and miR-98.

The miR-30 miRNA family includes miR-30a, miR-30b, miR-30c, miR-30d and miR-30e.

The miR-125 miRNA family includes miR-125a and miR-125b.

The miR-10 miRNA family includes miR-10a and miR-10b.

The miR-99 miRNA family includes miR-99a and miR-99b.

Expression of miRNAs selected from one, two, three, four or more miRNA families may be determined in order to diagnose non-melanoma skin cancer as described herein. For example, expression of miRNAs selected from one, two, three, four or all of the group consisting of let-7 miRNAs, miR-30 miRNAs, miR-125 miRNAs, miR-10 miRNAs and miR-99 miRNAs may determined.

The skilled person is readily able to employ suitable controls for use in the methods described herein. Suitable controls include cells, preferably keratinocytes, from healthy (i.e. non-lesional) skin which is not affected by the skin cancer. For example, cells from normal, healthy, non-sun-exposed skin from individuals without chronic skin disease or skin cancer. Control cells may be obtained from the same individual as the test sample cells, or a different individual, for example a healthy individual not suffering from or susceptible to skin cancer.

In some embodiments, an individual being assessed for a non-melanoma skin cancer as described herein may be immunosuppressed and may, for example, be the recipient of an organ transplant.

A suitable sample of skin cells may be taken from a lesion or other site on the skin of the individual which displays one or more symptoms of a skin cancer, such as abnormal keratinocyte proliferation or differentiation. In some embodiments, RNA may be isolated from the skin cells using methods well known in the art (see, e.g., Lagos-Quintana et al, Science 294:853-858 (2001); Grad et al, Mol Cell 11: 1253-1263 (2003); Mourelatos et al, Genes Dev 16:720-728 (2002); Lagos-Quintana et al, Curr Biol 12:735-739 (2002); Lagos-Quintana et al, RNA 9:175-179 (2003)). In other embodiments, miRNA expression may be determined directly, for example using in situ hybridisation.

The expression of a miRNA in a cell may be determined by measuring the amount of miRNA precursor or, more preferably the amount of mature miRNA, which is present in the cells.

The amount of miRNA in a cell may be conveniently measured by any convenient technique, including, for example, quantitative PCR, bead-based flow cytometry, microarrays, such as Taqman™ human miRNA array (Taqman low density array), northern blotting, dot blotting, RNase protection assays, primer extension analysis, miRNA specific in situ hybridization, and Invader™ assays. Suitable techniques are described in Liu et al. (2004); Thomson et al. (2004); Babak et al. (2004), Chen, Ridzon et al. (2005); Castoldi, Schmidt et al. (2006), Kim et al (2006); Kloosterman et al, Nature Methods, 3 (1), 27-29 (2006). Suitable reagents for miRNA specific in situ hybridization are commercially available (e.g. Exiqon A/S, Denmark).

miRNA expression may be determined in serum or plasma (“circulating” miRNAs). For example, RNA may be extracted from plasma/serum using standard techniques and miRNA expression measured by real time PCR.

miRNA levels may be measured in lymph nodes to detect the presence of metastasis.

In some embodiments, the expression of one or more miRNAs in a sample may be determined by microarray techniques. Microarrays generally comprise nucleic acid probes of different sequences immobilised in a predetermined arrangement on a solid support. Because different nucleic acid probes are immobilised at different locations on the support, the binding of a label which is observed at a particular location is indicative of specific binding to the nucleic acid probe immobilised at that location.

Microarrays may be synthesised using conventional techniques by synthesising nucleic acid probes and then attaching the probes to the support in a site-specific fashion, or by synthesising the nucleic acid probes in situ at predetermined locations on the support. Microarrays for use in the detection of human miRNAs are also commercially available (e.g. TaqMan® Human microRNA Array v1.0; Applied Biosystems, CA USA).

Conveniently, a Locked Nucleic Acid (LNA)-based miRNA microarray may be employed. Typically, total RNA is isolated from the sample skin cells, labelled and hybridized onto a microarray containing LNA (Locked Nucleic Acid)-modified probes for each known miRNA. The high affinity LNA technology provides the LNA Array with high sensitivity, high specificity and Tm-normalized probes. LNA microarrays are available commercially (e.g. miRCURY™, Exiqon).

In use, a microarray is contacted with a sample under conditions that promote specific binding of miRNAs in the sample to one or more of the immobilised nucleic acid molecules on the microarray. The miRNAs in the sample bind to one or more different locations on the microarray, via the nucleic acid molecules immobilised at those locations to produce a particular binding pattern. This binding pattern can then be detected by any convenient technique. For example, all nucleic acid molecules, including miRNA molecules, in the sample may be labelled with a suitable label, typically a fluorescent label, and the locations at which label is present on the microarray following exposure to the sample can be observed. Since the immobilised nucleic acid sequences have different predetermined locations on the microarray, the observed binding pattern is indicative of the presence and/or concentration of a particular miRNA in the sample. Techniques for detecting binding to microarrays are well known in the art (see for example, U.S. Pat. No. 5,763,870, U.S. Pat. No. 5,945,679 and U.S. Pat. No. 5,721,435).

A method of determining the expression of one or more miRNAs may, for example, comprise: a) contacting a sample with a microarray comprising immobilised probes for said one or more miRNAs under conditions sufficient for specific binding to occur between the miRNA and its corresponding immobilised probe; and b) interrogating the microarray to determined the presence or amount of binding of one or more miRNAs in the sample.

In some embodiments, the expression of one or more miRNAs in a sample may be determined by bead-based flow cytometry methods such as FlexmiR™ (Exiqon A/S, Copenhagen) (Lu et al Nature 2005 435 834-838). This involves marking individual beads with fluorescence tags, each representing a single miRNA, and coupling the beads to probes that are complementary to miRNAs of interest. miRNAs are ligated to 5′ and 3′ adaptors, reverse-transcribed, amplified by PCR using a common biotinylated primer, hybridized to the capture beads, and stained with a suitable reagent such as streptavidin-phycoerythrin. The beads are then analyzed using a flow cytometer capable of measuring bead color (denoting miRNA identity) and phycoerythrin intensity (denoting miRNA abundance). Because hybridization takes place in solution, bead-based flow cytometry methods may allow more specific detection of closely related miRNAs than microarray techniques.

In some embodiments, the expression of one or more miRNAs in a sample may be determined by miRNA-specific quantitative real-time PCR. For this, total RNA is isolated from the skin biopsy, reverse transcribed using miRNA-specific stem-loop primers, and then amplified by real-time PCR, for example using TaqMan® probes. The assays target only mature microRNAs, not their precursors, ensuring biologically relevant results. Techniques for real-time PCR are well known in the art (Livak et al PCR Methods Appl (1995) 4 357-362) and reagents for use in such techniques are commercially available (e.g. Applied Biosystems, CA USA).

Following assessment of a non-melanoma skin cancer, such as SCC or BCC, by a method described herein, the individual may be treated for the condition.

A method of treating a skin cancer as described herein may comprise;
assessing an individual for a non-melanoma skin cancer using a method described above and;
administering a therapy for the skin cancer.

Other aspects of the invention provide an anti-cancer agent for use in a method of treatment of non-melanoma skin cancer in an individual which comprises assessing a non-melanoma skin cancer in the individual using a method described above and the use of an anti-cancer agent in the manufacture of a medicament for use in a method of treatment of non-melanoma skin cancer in an individual which comprises assessing a non-melanoma skin cancer in the individual using a method described above.

Therapies for skin cancer include surgical techniques, such as curettage, electrodessication, cryosurgery, surgical excision and Mohs micrographic surgery, or non-surgical techniques, such as radiotherapy, topical and injectable chemotherapy, for example with anti-cancer agents such as 5-fluorouracil, capecitabine, celecoxib, retinoids such as acitretin, isotretinoin, tazarotene, imiquimod, or IFNalpha, and photodynamic therapy, for example with 5-aminolevulinate.

Methods of assessing a non-melanoma skin cancer as described herein may also be useful in determining the responsiveness of an individual to a therapy for the non-melanoma skin cancer, such as BCC or SCC. A method of assessing the efficacy of a therapy for a non-melanoma skin cancer in an individual or the responsiveness of an individual to a therapy for a non-melanoma skin cancer may comprise:
determining the expression of one or more of the miRNAs set out above in one or more cells obtained from an individual subjected to a regimen of treatment with the non-melanoma skin cancer.

A control tissue sample may be obtained before the regimen of therapy for the non-melanoma skin cancer is initiated. A change, for example, an increase or decrease in expression of one or more of the miRNAs set out above after initiation of the therapy regimen may be indicative that the regimen normalises miRNA levels in cells and is therefore efficacious for the treatment of the individual.

The absence of any change in the expression of the one or more of the miRNAs set out above after initiation of the regimen of therapy may be indicative that the regimen is not efficacious for the treatment of the individual.

The expression of the one or more of the miRNAs may be measured in samples obtained at one or more, two or more, or three or more time points during or after the treatment. The amount of change in the expression of the one or more of the miRNAs may be indicative of the level of responsiveness of the individual to the regimen.

Suitable therapies for a non-melanoma skin cancer are described above.

A treatment regimen is a predetermined scheme or program which defines the parameters of the treatment to which the individual is to be subjected. For example, the regimen may set out the dosage, the mode of administration and the timetable or schedule of administration of the cancer therapy with which the individual is to be treated.

An appropriate regimen of treatment with a cancer therapy can vary from patient to patient. Determining the appropriate dosage, mode and schedule of administration will generally involve the balancing of the level of therapeutic benefit against any risk or deleterious side effects of the treatments. For example, the initial dosage level and schedule will depend on a variety of factors including, but not limited to, the activity of the particular cancer therapy, the chosen route of administration, the time of administration, the rate of excretion of the compound, the duration of the treatment, other drugs, compounds, and/or materials used in combination, and the age, sex, weight, condition, general health, and prior medical history of the individual. The parameters of the regimen may be optimised for an individual using the methods described below.

The initial treatment regimen will ultimately be at the discretion of the physician, although generally the dosage and other parameters will be selected in order to achieve therapeutic benefit as assessed using the methods described herein, without causing substantial harmful or deleterious side-effects.

In the absence of any change in the expression of the one or more miRNAs, the regimen may be altered, for example by increasing the dosage, frequency of administration and/or duration of treatment, and the responsiveness of the individual to the altered regimen determined. This may be repeated until a change in the cancer therapy is observed. A treatment regimen which alters the expression of the one or more miRNAs may be identified.

In some embodiments, a treatment regimen which produces a change in the expression of the one or more miRNAs may be altered, for example, by increasing the dosage, frequency of administration and/or duration of treatment, and the responsiveness of the individual to the altered regimen determined. This may be repeated until no further change in the expression of the one or more miRNAs is observed. A treatment regimen which produces a maximal change in the expression of the one or more miRNAs with acceptable toxicity levels may be identified.

In some embodiments, following identification of a treatment regimen which changes the expression of the one or more miRNAs or produces a maximal change in the expression of the one or more miRNAs, the safety, tolerability and/or pharmacokinetic effects of the regimen may be assessed in one or more individuals.

The progress of a cancer therapy regimen may be monitored in an individual using the methods described herein, for example to ensure that the pharmacological effect is sustained in the individual throughout the duration of the treatment. A method for monitoring the treatment of a non-melanoma skin cancer in individual with a cancer therapy may comprise:
(a) subjecting the individual to a regimen of cancer therapy; and
(b) monitoring in one or more cells of the individual the expression of the one or more miRNAs during said treatment.

The expression of the one or more miRNAs may be monitored by periodically obtaining samples from the individual and measuring the expression of the one or more miRNAs in the samples obtained.

A change in the expression of the one or more miRNAs in response to the regimen is indicative that the regimen is effective for therapy in the individual. The change may be sustained over the duration of the regimen, for example, because miRNA levels remain above or below a predetermined value or within a predetermined range of values throughout the treatment.

A regimen which is found to be not fully effective may be altered, for example by altering the dosage or schedule, to restore the change in the expression of the one or more miRNAs; for example, by restoring levels of the one or more miRNAs to above or below a predetermined value or within a predetermined range of values.

Other aspects of the invention relate to methods of treatment of skin cancer in an individual.

A method of treatment of a non-melanoma skin cancer, such as BCC or SCC, in an individual may comprise;
increasing or reducing the expression or activity of one or more miRNAs selected from the group consisting of the miRNAs listed in Table 1 in skin cancer cells of the individual.

For example, the expression or activity of one or more miRNAs selected from the group consisting of the miRNAs listed in Table 2 in skin cancer cells of the individual may be reduced.

In some embodiments, a method of treatment of SCC may comprise;
reducing the expression or activity of one or more miRNAs selected from the group consisting of the miRNAs listed in Table 4a or 4b in skin cancer cells of the individual.

For example, the expression or activity of miR-21 and/or miR-31 may be reduced.

In some embodiments, a method of treatment of BCC may comprise;
reducing the expression or activity of one or more miRNAs selected from the group consisting of the miRNAs listed in Tables 6a and/or 6b in skin cancer cells of the individual.

For example, the expression or activity of miR-424 and/or miR-514 may be reduced.

The expression or activity of a target miRNA may be reduced by decreasing in total amount of the target miRNA in the cell or by decreasing the amount of the target miRNA which is present in the cell in an active form.

In some embodiments, the expression or activity of the target miRNA may be reduced by administering a therapeutically effective amount of a miRNA inhibitor to an individual in need thereof.

An inhibitor of a target miRNA is a compound which reduces or represses the activity or expression of the target miRNA. Preferably, the inhibitor has no effect or substantially no effect on non-target miRNAs. Suitable inhibitors may be readily designed by the skilled person from the sequence of the target miRNA. Sequences of target miRNAs are available from the miRNA Registry and are set out in Table 16.

Suitable inhibitors may include single or double stranded oligonucleotides which are able to bind to mature miRNA or its precursor forms and inhibit the activity of mature miRNA, prevent or inhibit its production or increase its rate of depletion. Suitable oligonucleotides may be oligodeoxyribonucleotides, oligoribonucleotides or modified oligonucleotides as described below

In some embodiments, the activity of a mature miRNA may be inhibited by the binding of a single stranded oligonucleotide which has a sequence which is sufficiently complementary to the sequence of the miRNA to hybridise to the target miRNA by Watson-Crick base-pairing. The use of such ‘antisense’ oligonucleotides is well-established in the art.

Oligonucleotides may be generated in vitro or ex vivo for administration or anti-sense RNA may be generated in vivo within cells in which inhibition is desired. Thus, double-stranded DNA may be placed under the control of a promoter in a “reverse orientation” such that transcription of the anti-sense strand of the DNA yields RNA which is complementary to the precursor miRNA. The complementary anti-sense RNA sequence may then bind with the target miRNA, inhibiting its cellular activity (see for example, Applied Antisense Oligonucleotide Technology C A. Stein (1998) Wiley & Sons).

A suitable oligonucleotide for inhibition of an miRNA may have about 10 to 30 nucleotides, preferably about 20 nucleotides e.g. 14-23 nucleotides, for example about 15, 16 or 17.

The construction of anti-sense sequences and their use is well known in the art and is described for example in Peyman and Ulman, Chemical Reviews, 90:543-584, (1990) and Crooke, Ann. Rev. Pharmacol. Toxicol. 32:329-376, (1992).

Nucleotides comprise a base portion, generally a heterocyclic base such as a purine or pyrimidine, which is covalently linked to a sugar group, typically a pentofuranosyl sugar, which further comprises a phosphate group. The phosphate group is generally linked to the 2′, 3′ or 5′ hydroxyl moiety of the sugar. The phosphate groups covalently link adjacent nucleotides to one another to form an oligonucleotide. Within the oligonucleotide structure, the phosphate groups are commonly referred to as forming the internucleotide backbone of the oligonucleotide. The normal linkage or backbone of RNA and DNA is a 3′ to 5′ phosphodiester linkage

Single-stranded oligonucleotides for the inhibition of miRNA activity may be chemically modified. Modified oligonucleotides are described in more detail below.

Examples of modified oligonucleotides which may be used to inhibit target miRNA molecules include LNA Knockdown probes, (Orom, Kauppinen et al. 2006), 2′-O-methyl modified RNA oligonucleotides (Cheng, Byrom et al. 2005), and “antagomirs” (Krutzfeldt, Rajewsky et al. 2005 Mattes et al 2007).

Antagomirs are chemically modified, single-stranded RNA analogues conjugated to cholesterol. An antagomir typically comprises at least 19 nucleotides which are complementary to the sequence of a target miRNA which allow hybridisation between the antagomir and the target miRNA, thereby inhibiting the activity of the miRNA target. Antagomirs can discriminate between single nucleotide mismatches of the targeted miRNA and have been shown to silence specific miRNAs in vivo (Krutzfeldt, Rajewsky et al. 2005). Antagomirs have also been shown to efficiently target miRNAs when injected locally into the mouse cortex (Krutzfeldt, Kuwajima et al. 2007).

Other useful inhibitors include oligonucleotides which cause inactivation or cleavage of mature miRNA or its precursor forms. Suitable oligonucleotides may be chemically modified, or have enzyme activity, which causes cleavage of a nucleic acid at a specific site
thus influencing activity of miRNAs. Examples include ribozymes, EDTA-tethered oligonucleotides, or covalently bound oligonucleotides, such as a psoralen or other cross-linking reagent-bound oligonucleotides. Background references for ribozymes include Kashani-Sabet and Scanlon, 1995, Cancer Gene Therapy, 2(3): 213-223, and Mercola and Cohen, 1995, Cancer Gene Therapy, 2(1), 47-59.

In some embodiments, the activity of a mature miRNA may be inhibited using a double-stranded oligonucleotide which comprises a sequence which is complementary to a target miRNA. A suitable double-stranded oligonucleotide may comprise about 10 to 30 nucleotides, preferably about 20 nucleotides e.g. 18-23 nucleotides. Techniques for inhibiting target miRNAs using double-stranded inhibitory oligonucleotides are known in the art (Soutschek, J. et al Nature 432, 173-178 (2004), Vermeulen, Robertson et al. 2007 and US20050182005).

Other useful inhibitors include double- or single-stranded DNA or double- or single-stranded RNA “aptamers” that bind to specific targets via interactions other than Watson-Crick base pairing. Suitable oligonucleotides (e.g., RNA oligonucleotides) that bind a specific miRNA can be generated using the techniques of SELEX (Tuerk, 1997, Methods Mol Biol 67, 2190). In this technique, a very large pool (10⁶-10⁹) of random sequence nucleic acids is bound to the target using conditions that cause a large amount of discrimination between molecules with high affinity and low affinity for binding the target. The bound molecules are separated from unbound, and the bound molecules are amplified by virtue of a specific nucleic acid sequence included at their termini and suitable amplification reagents. This process is reiterated several times until a relatively small number of molecules remain that possess high binding affinity for the target. These molecules can then be tested for their ability to modulate miRNA activity as described herein.

A modified oligonucleotide may contain one or more modified backbone linkages. Backbone linkages in a modified oligonucleotide may include, for example, non-phosphodiester linkages, such as phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogues of these, and those having inverted polarity wherein the adjacent pairs of nucleoside units are linked 3′-5′ to 5′-3′ or 2′-5′ to 5′-2′. Modified oligonucleotides may comprise linkages which lack phosphate groups and may comprise short chain alkyl or cycloalkyl internucleoside linkages, mixed heteroatom and alkyl or cycloalkyl internucleoside linkages, or one or more short chain heteroatomic or heterocyclic internucleoside linkages, for example morpholino; siloxane; sulfide, sulfoxide, sulfone; formacetyl; thioformacetyl; methylene formacetyl; thioformacetyl; alkene containing; sulfamate; methyleneimino; methylenehydrazino; sulfonate; sulfonamide; amide; or other linkages comprising N, O, S and/or CH₂ groups.

Suitable modified oligonucleotides may comprise phosphorothioate backbones or heteroatom backbones, and in particular —CH₂—NH—O—CH₂—, —CH₂—N(CH.sub.3)-O—CH₂—, —CH.sub.2-O—N(CH₃)—CH₂—, CH₂—N(CH₃)—N(CH₃)—CH₂— and —O—N(CH₃)—CH₂—CH₂—[wherein the native phosphodiester backbone is represented as —O—P—O—CH₂—].

Modified oligonucleotides may also contain one or more substituted sugar moieties. Suitable sugar moieties may comprise one of the following at the 2′ position: OH; F; O—, S—, or N-alkyl; O—, S—, or N-alkenyl; O—, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly suitable are O[(CH₂)ₙO]ₘCH₃, O[(CH₂)ₙO]ₘOCH₃, O[(CH₂)ₙO]ₘNH₂, O[(CH₂)ₙ]ₘCH₃, O[(CH₂)ₙO]ₘONH₂ and O[(CH₂)ₙO]ₘON(CH₂)ₙCH₃)]₂, where n and m are from 1 to about 10.

Modified sugar moieties may comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF, OCF, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Suitable modifications include 2′-methoxyethoxy (2′-O—CH₂CH₂OCH₃, also known as 2′-O-(2-methoxyethyl) or 2′-MOE) (Martin et al., Helv. Chim. Acta, 1995, 78, 486 504) i.e. an alkoxyalkoxy group, 2′-dimethylaminooxyethoxy, i.e., a O(CH₂)₂ON(CH₃)₂ group, also known as 2′-DMAOE, 2′-methoxy (2′-O—CH₃), 2′-aminopropoxy (2′-OCH₂CH₂CH₂ NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide.

Modified oligonucleotides may also contain one or more sugar mimetics instead of a pentofuranosyl sugar. Suitable sugar mimetics include cyclobutyl moieties, azido-ribose, carbocyclic sugar analogues a-anomeric sugars; epimeric sugars such as arabinose, xyloses or lyxoses, pyranose sugars, furanose sugars, and sedoheptulose.

Modified oligonucleotides may also include base modifications or substitutions. Modified nucleotide bases can be used instead of or in addition to the naturally occurring bases i.e. the purine bases adenine (A) and guanine (G), and the pyrimidine bases thymine (T), cytosine (C) and uracil (U). For example, modified bases may increase the stability of the molecule. Modified bases known in the art include alkylated purines and pyrimidines, acylated purines and pyrimidines, and other heterocycles. These classes of pyrimidines and purines are known in the art and include pseudoisocytosine, N4,N4-ethanocytosine, 8-hydroxy-N6-methyladenine, 4-acetylcytosine, 5-(carboxyhydroxylmethyl) uracil, 5 fluorouracil, 5-bromouracil, 5-carboxymethylaminomethyl-2-thiouracil, 5-carboxymethylaminomethyl uracil, dihydrouracil, inosine, N6-isopentyl-adenine, 1-methyladenine, 1-methylpseudouracil, 1-methylguanine, 2,2-dimethylguanine, 2-methyladenine, 2-methylguanine, 3-methylcytosine, 5-methylcytosine, N6-methyladenine, 7-methylguanine, 5-methylaminomethyl uracil, 5-methoxy amino methyl-2-thiouracil, -D-mannosylqueosine, 5-methoxycarbonylmethyluracil, 5methoxyuracil, 2 methylthio-N6-isopentenyladenine, uracil-5-oxyacetic acid methyl ester, psueouracil, 2-thiocytosine, 5-methyl-2 thiouracil, 2-thiouracil, 4-thiouracil, 5-methyluracil, N-uracil-5-oxyacetic acid methylester, uracil 5-oxyacetic acid, queosine, 2-thiocytosine, 5-propyluracil, 5-propylcytosine, 5-ethyluracil, 5ethylcytosine, 5-butyluracil, 5-pentyluracil, 5-pentylcytosine, and 2,6,diaminopurine, methylpsuedouracil, 1-methylguanine and 1-methylcytosine.

In some embodiments, both the sugar and the backbone linkage of one or more, preferably all of the nucleotides in a modified oligonucleotide may be replaced with non-natural groups. The bases are maintained for hybridization with the target miRNA. Suitable modified oligonucleotides may include peptide nucleic acids (PNA). In PNA, the oligonucleotide sugar-backbone is replaced with an amide containing backbone, in particular an aminoethylglycine backbone.

The bases are retained and are bound directly or indirectly to aza-nitrogen atoms of the amide portion of the backbone.

Modified oligonucleotides may be chemically linked to one or more moieties or groups which enhance the activity, cellular distribution or cellular uptake of the oligonucleotide. Suitable moieties include lipid moieties such as cholesterol, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethyl-ammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety.

miRNA inhibitors may be transferred into the cell using a variety of techniques well known in the art. For example, oligonucleotide inhibitors can be delivered into the cytoplasm without specific modification. Alternatively, they may be delivered by the use of liposomes which fuse with the cellular membrane or are endocytosed, i.e. by employing ligands such as antibodies which are attached to the liposome or directly to the oligonucleotide and which bind to surface membrane protein receptors of the cell, resulting in endocytosis. Alternatively, the cells may be permeabilized to enhance transport of the oligonucleotides into the cell, without injuring the host cells or a DNA binding protein, e.g. HBGF-1, which transports oligonucleotides into a cell may be employed.

A method of treatment of a skin cancer in an individual may comprise;
increasing the amount or activity of one or more miRNAs selected from the group consisting of the miRNAs listed in Table 3 in skin cancer cells of the individual.

In some embodiments, a method of treatment of SCC in an individual may comprise;
increasing the amount or activity of one or more miRNAs selected from the group consisting of the miRNAs listed in Table 9a and/or Table 9b in skin cancer cells of the individual.

For example, a method may comprise increasing the activity or expression of 1, 2, 3, 4, 5, 6, 7, 8, or 9 or more miRNAs from the group consisting of the ten highest scoring miRNAs shown in Table 9b and, optionally, one or more additional miRNAs listed in Tables 9a and/or 9b.

Preferably, the amount or activity of one or more miRNAs selected from the group consisting of the miRNAs listed in Table 5 may be increased in skin cancer cells of the individual. For example, the amount or activity of miR-125b, miR-15, and/or a let-7 family miRNA, and optionally one or more additional miRNAs listed in Table 5 may be increased.

In some embodiments, a method of treatment of BCC in an individual may comprise;
increasing the amount or activity of one or more miRNAs selected from the group consisting of the miRNAs listed in Tables 10a and/or 10b in skin cancer cells of the individual.

For example, a method may comprise increasing the activity or expression of 1, 2, 3, 4, 5, 6, 7, 8, or 9 or more miRNAs from the group consisting of the ten highest scoring miRNAs shown in Table 10b and, optionally, one or more additional miRNAs listed in Tables 10a and/or 10b.

Preferably, the amount or activity of one or more miRNAs selected from the group consisting of the miRNAs listed in Table 7 may be increased in skin cancer cells of the individual. For example, the amount or activity of miR-203, miR-15b, miR-16 and/or miR-193a and optionally one or more additional miRNAs listed in table 7 may be increased.

The expression or activity of a target miRNA may be increased by administering to an individual in need thereof a therapeutically effective amount of;
(i) the target miRNA or a precursor thereof,
(ii) a nucleic acid encoding the target miRNA or a precursor thereof,
(iii) an analogue, derivative or modified form of the target miRNA which retains activity.

Nucleic acid sequences encoding a target miRNA or a target miRNA precursor may be comprised within a vector. Suitable vectors can be chosen or constructed, containing appropriate regulatory sequences which will drive transcription in the target cell, including promoter sequences, terminator fragments, polyadenylation sequences, enhancer sequences, marker genes and other sequences as appropriate. A vector may comprise a selectable marker to facilitate selection of the transgenes under an appropriate promoter. For further details see, for example, Molecular Cloning: a Laboratory Manual: 3rd edition, Sambrook & Russell, 2001, Cold Spring Harbor Laboratory Press.

Many known techniques and protocols for manipulation of nucleic acid, for example in preparation of nucleic acid constructs, mutagenesis, sequencing, introduction of DNA into cells and gene expression, and analysis of proteins, are described in detail in Protocols in Molecular Biology, Second Edition, Ausubel et al. eds. John Wiley & Sons, 1992.

A nucleic acid vector may be introduced into a host cell, for example a lesional skin cell. Suitable techniques for transporting the constructor vector into the cell are well known in the art and include calcium phosphate transfection, DEAE-Dextran, electroporation, liposome-mediated transfection and transduction using retrovirus or other virus, e.g. vaccinia or lentivirus.

The particular choice of a transformation technology will be determined by its efficiency to transform the particular host cells employed as well as the experience and preference of the operator with a particular methodology of choice.

An analogue, derivative or modified form of a miRNA retains the biological activity of the mature miRNA (i.e. a miRNA agonist) and may be an oligoribonucleotide or oligodeoxyribonucleotide with one or more modifications which improve the stability, transport or other pharmacological properties. Suitable modifications include modifications to the backbone linkages, bases or sugar moieties of one or more of the constituent nucleotides and are described in more detail above.

The term “treatment” in the context of treating a skin cancer, pertains generally to treatment and therapy, whether of a human or an animal (e.g. in veterinary applications), in which some desired therapeutic effect is achieved, for example, the inhibition of the progress of the disorder, and includes a reduction in the rate of progress, a halt in the rate of progress, amelioration of the disorder, and cure of the disorder. Treatment as a prophylactic measure (i.e. prophylaxis) is also included.

While it is possible for an active compound such as an miRNA agonist or antagonist as described above, to be administered alone, it is preferable to present it as a pharmaceutical composition (e.g., formulation) comprising at least one active compound, as defined above, together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents.

A composition may comprise multiple active compounds as described above (i.e. miRNA agonists or antagonists) to increase or decrease the amount or activity of multiple miRNA targets in a skin cancer cell.

Pharmaceutical compositions comprising a miRNA agonist or antagonist as defined above, for example, admixed or formulated together with one or more pharmaceutically acceptable carriers, excipients, buffers, adjuvants, stabilisers, or other materials, as described herein, may be used in the methods described herein.

The term “pharmaceutically acceptable” as used herein pertains to compounds, materials, compositions, and/or dosage forms which are, within the scope of sound medical judgement, suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Suitable carriers, excipients, etc. can be found in standard pharmaceutical texts, for example, Remington's Pharmaceutical Sciences, 18th edition, Mack Publishing Company, Easton, Pa., 1990.

The formulations may conveniently be presented in unit dosage form and may be prepared by any methods well-known in the art of pharmacy. Such methods include the step of bringing the active compound into association with a carrier which may constitute one or more accessory ingredients. In general, the formulations are prepared by uniformly and intimately bringing into association the active compound with liquid carriers or finely divided solid carriers or both, and then if necessary shaping the product.

Formulations may be in the form of liquids, solutions, suspensions, emulsions, elixirs, syrups, tablets, lozenges, granules, powders, capsules, cachets, pills, ampoules, suppositories, pessaries, ointments, gels, pastes, creams, sprays, mists, foams, lotions, oils, boluses, electuaries, or aerosols.

The miRNA agonist or antagonist (s) or pharmaceutical composition comprising the miRNA agonist or antagonist (s) may be administered to a subject by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); parenteral, for example, by injection.

In preferred embodiments, an active compound is administered directly at the site of action by topical administration to lesional skin cells.

Formulations suitable for topical administration (e.g. transdermal, intranasal, ocular, buccal, and sublingual) may be formulated as an ointment, cream, suspension, lotion, powder, solution, past, gel, spray, aerosol, or oil. Alternatively, a formulation may comprise a patch or a dressing such as a bandage or adhesive plaster impregnated with active compounds and optionally one or more excipients or diluents.

In some embodiments, miRNA agonist(s) or antagonist(s) as described herein may be administered in combination with other skin cancer therapies. Skin cancer therapies are described in more detail above.

Other aspects of the invention relate to screening for compounds useful in the treatment of skin cancers.

A method of screening for a compound useful in the treatment of a skin cancer, such as SCC or BCC, may comprise;
contacting a cell with a test compound and;
determining the expression of one or more miRNAs selected from the group consisting of the miRNAs listed in Table 1 relative to controls,
wherein an increase or decrease in expression in the presence of the test compound is indicative that the compound is useful in the treatment of an skin cancer.

In some embodiments, expression of one or more microRNAs selected from the group consisting of one or more of the miRNAs selected from the group consisting of the miRNAs listed in Table 2 may be determined in the cell,
wherein a decrease in expression in the presence of the test compound relative to its absence is indicative that the compound is useful in the treatment of an skin cancer.

In some embodiments, expression of one or more microRNAs selected from the group consisting of the miRNAs listed in Table 3 may be determined in the cell,
wherein an increase in expression of the one or more microRNAs in the presence of the test compound relative to its absence is indicative that the compound is useful in the treatment of a skin cancer.

A method of screening for a compound useful in the treatment of SCC may comprise;
contacting a cell with a test compound and;
determining the expression of one or more miRNAs selected from the group consisting of the miRNAs listed in Table 4a or 4b in the cell,
wherein a decrease in expression in the presence of the test compound relative to its absence is indicative that the compound is useful in the treatment of SCC.

A method of screening for a compound useful in the treatment of SCC may comprise
contacting a cell with a test compound and;
determining the expression of one or more miRNAs selected from the group consisting of the miRNAs listed in Table 9a or 9b in the cell,
wherein an increase in expression in the presence of the test compound relative to its absence is indicative that the compound is useful in the treatment of SCC.

For example, a method may comprise determining the expression of 1, 2, 3, 4, 5, 6, 7, 8, or 9 or more miRNAs from the group consisting of the ten highest scoring miRNAs shown in Table 9b and, optionally, one or more additional miRNAs listed in Tables 9a and/or 9b.

Preferably, the one or more miRNAs are selected from the group consisting of the miRNAs listed in Table 5. For example, the expression of miR-125b, miR-15, and/or a let-7 family miRNA may be determined.

A method of screening for a compound useful in the treatment of BCC may comprise;
contacting a cell with a test compound and;
determining the expression of one or more microRNAs selected from the group consisting of one or more miRNAs selected from the group consisting of the miRNAs listed in Table 6a and/or 6b,
wherein a decrease in expression in the presence of the test compound relative to its absence is indicative that the compound is useful in the treatment of BCC.

For example, the expression or activity of miR-424 and/or miR-514 may be determined.

A method of screening for a compound useful in the treatment of BCC may comprise;
determining the expression of one or more miRNAs selected from the group consisting of one or more miRNAs selected from the group consisting of the miRNAs listed in Table 10a and/or 10b in the cell,
wherein an increase in expression in the presence of the test compound relative to its absence is indicative that the compound is useful in the treatment of treatment of BCC.

For example, a method may comprise determining the expression of 1, 2, 3, 4, 5, 6, 7, 8, or 9 or more miRNAs from the group consisting of the ten highest scoring miRNAs shown in Table 10b and, optionally, one or more additional miRNAs listed in Tables 10a and/or 10b.

Preferably, one or more miRNAs is selected from the group consisting of the miRNAs listed in Table 7. For example, the amount or activity of miR-203, miR-15b, miR-16 and/or miR-193a and optionally one or more additional miRNAs listed in table 7 may be determined.

Techniques for determining the amount of expression of a target miRNA in a cell are described in more detail above.

The cell is contacted with the test compound in vitro and may be an isolated cell, for example a cell from a cultured cell line or may be comprised in or obtained from a tissue sample which is obtained from an individual.

Suitable cells for use in the present methods may be higher eukaryotic cells, preferably mammalian cells, such as human cells. The cell may be a human skin cell, for example a keratinocyte. In some embodiments, the cell may be a skin cancer cell, for example a skin cancer cell from a biopsy or a primary tissue culture or a skin cancer cell from a cultured cell line.

The precise format for performing the methods described herein may be varied by those of skill in the art using routine skill and knowledge.

Compounds which may be screened using the methods described herein may be natural or synthetic chemical compounds used in drug screening programmes. Extracts of plants, microbes or other organisms which contain several characterised or uncharacterised components may also be used.

Combinatorial library technology provides an efficient way of testing a potentially vast number of different compounds for ability to modulate an interaction. Such libraries and their use are known in the art, for all manner of natural products, small molecules and peptides, among others. The use of peptide libraries may be preferred in certain circumstances.

In some embodiments, the test compound may be an analogue, variant or derivative of a target miRNA as described above.

The amount of test compound or compound which may be added to a method of the invention will normally be determined by serial dilution experiments. Typically, from about 0.001 nM to 1 mM or more of putative inhibitor compound may be used, for example from 0.01 nM to 100 μM, e.g. 0.1 to 50 μM, such as about 10 μM.

In some embodiments, a method may comprise identifying the test compound as a miRNA inhibitor or antagonist as described above. Such a compound may, for example, be useful in reducing the expression and/or activity of the target miRNA, for example in the treatment of a skin cancer, as described herein.

In other embodiments, a method may comprise identifying the test compound as an agonist (i.e. a promoter or enhancer) of a miRNA described above. Such a compound may, for example, be useful in increasing the expression and/or activity of the target miRNA, for example in the treatment of skin cancer, as described herein.

A test compound identified using one or more initial screens as having ability to modulate the expression and/or activity of one or more target miRNAs, may be assessed further using one or more secondary screens. A secondary screen may, for example, involve testing for a biological function such as an effect on skin lesions in an animal model of a skin cancer.

The test compound may be isolated and/or purified or alternatively, it may be synthesised using conventional techniques of recombinant expression or chemical synthesis. Furthermore, it may be manufactured and/or used in preparation, i.e. manufacture or formulation, of a composition such as a medicament, pharmaceutical composition or drug. These may be administered to individuals for the treatment of a skin cancer. Methods of the invention may thus comprise formulating the test compound in a pharmaceutical composition with a pharmaceutically acceptable excipient, vehicle or carrier for therapeutic application, as discussed further below.

Following identification of a compound which inhibits the expression or activity of a target miRNA described herein and which may therefore be useful in treating a skin cancer, a method may further comprise modifying the compound to optimise the pharmaceutical properties thereof.

The modification of a ‘lead’ compound identified as biologically active is a known approach to the development of pharmaceuticals and may be desirable where the active compound is difficult or expensive to synthesise or where it is unsuitable for a particular method of administration, e.g. peptides are not well suited as active agents for oral compositions as they tend to be quickly degraded by proteases in the alimentary canal. Modification of a known active compound (for example, to produce a mimetic) may be used to avoid randomly screening large number of molecules for a target property.

Modification of a ‘lead’ compound to optimise its pharmaceutical properties commonly comprises several steps. Firstly, the particular parts of the compound that are critical and/or important in determining the target property are determined. In the case of a peptide, this can be done by systematically varying the amino acid residues in the peptide, e.g. by substituting each residue in turn. These parts or residues constituting the active region of the compound are known as its “pharmacophore”.

Once the pharmacophore has been found, its structure is modelled according its physical properties, e.g. stereochemistry, bonding, size and/or charge, using data from a range of sources, e.g. spectroscopic techniques, X-ray diffraction data and NMR.

Computational analysis, similarity mapping (which models the charge and/or volume of a pharmacophore, rather than the bonding between atoms) and other techniques can be used in this modelling process.

In a variant of this approach, the three-dimensional structure of the compound which modulates the expression and/or activity of a target miRNA described herein is modelled. This can be especially useful where the compound changes conformation, allowing the model to take account of this in the optimisation of the lead compound.

A template molecule is then selected, onto which chemical groups that mimic the pharmacophore can be grafted. The template molecule and the chemical groups grafted on to it can conveniently be selected so that the modified compound is easy to synthesise, is likely to be pharmacologically acceptable, and does not degrade in vivo, while retaining the biological activity of the lead compound. The modified compounds found by this approach can then be screened to see whether they have the target property, or to what extent they exhibit it. Modified compounds include mimetics of the lead compound.

Further optimisation or modification can then be carried out to arrive at one or more final compounds for in vivo or clinical testing.

As described above, a compound identified and/or obtained using the present methods may be formulated into a pharmaceutical composition.

Pharmaceutical compositions are described in more detail above.

Various further aspects and embodiments of the present invention will be apparent to those skilled in the art in view of the present disclosure.

All documents mentioned in this specification and the miRNA registry entries for all the miRNAs mentioned are incorporated herein by reference in their entirety.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

Certain aspects and embodiments of the invention will now be illustrated by way of example and with reference to the figures and tables described below.

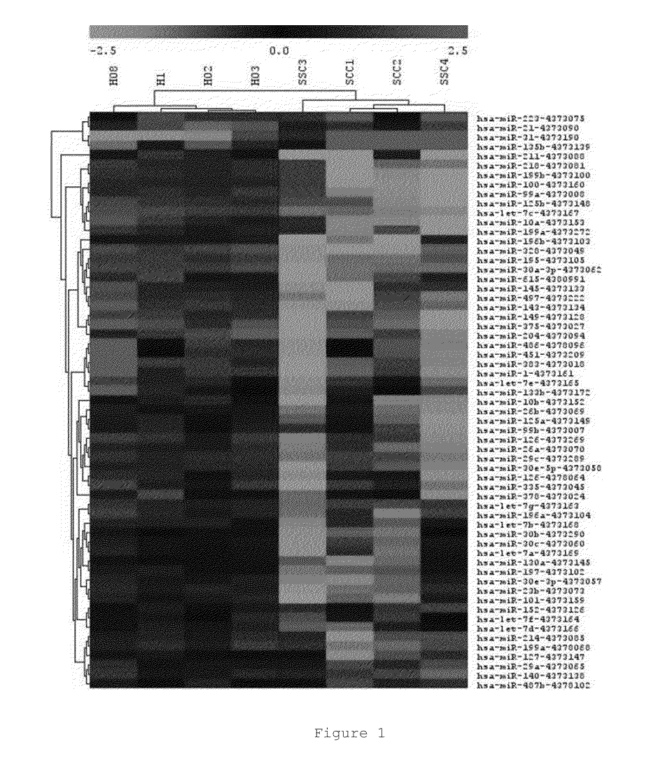

FIG. 1 shows a heatmap showing the results of unsupervised hierarchical clustering (Euclidian distance, average linkage) on a subset of 62 genes that were differentially expressed (FDR<2.5%) between healthy skin and SCC as determined by SAM analysis. Heatmap colors represent relative miRNA expression. A median expression value equal to 1 was designated black; dark grey, increased expression; light grey, reduced expression. Note that the color scale is logarithmic (i.e. 2 means 4-fold change, 0 means no change).

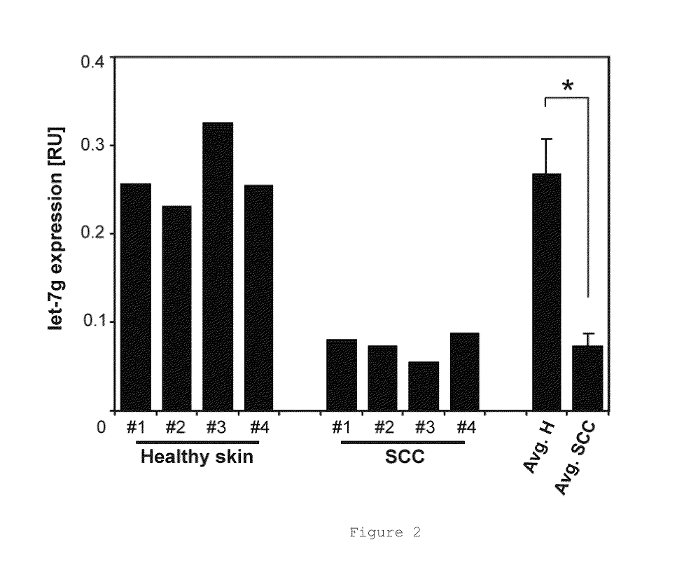

FIG. 2 shows the suppression of let-7g in SCC. P<0.001.

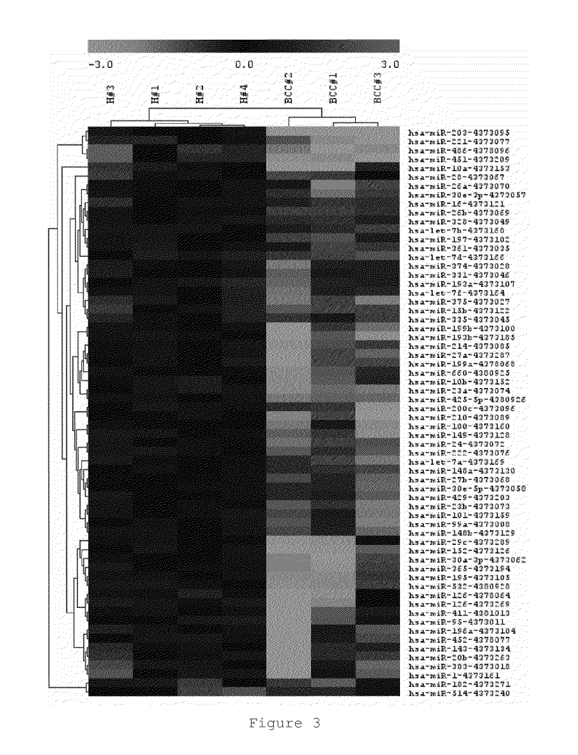

FIG. 3 shows a heatmap showing the results of unsupervised hierarchical clustering (Euclidian distance, average linkage) on a subset of 64 genes that were differentially expressed (FDR<2.5%) between healthy skin and BCC as determined by SAM analysis. Heatmap colors represent relative miRNA expression. A median expression value equal to 1 was designated black; dark grey, increased expression; light grey, reduced expression. Note that the color scale is logarithmic (i.e. 2 means 4-fold change, 0 means no change).

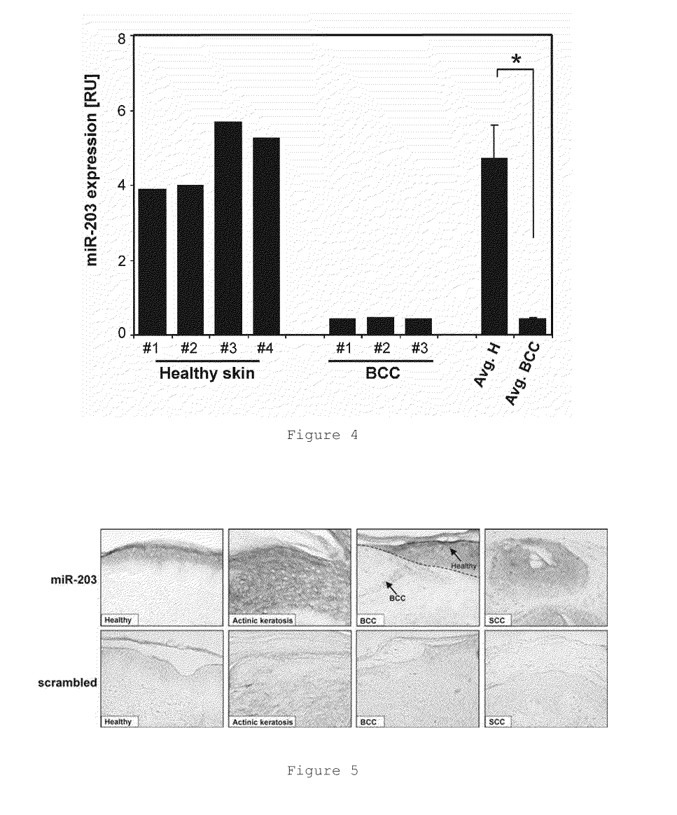

FIG. 4 shows the suppression of miR-203 in BCC. P<0.01.

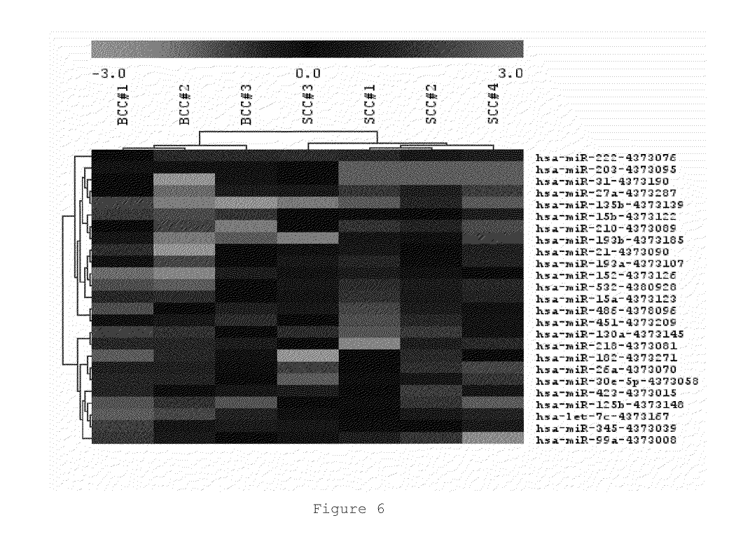

FIG. 5 shows the results of in situ hybridizations for miR-203 in samples of healthy skin, BCC, actinic keratosis, SCC. Specific LNA probes for miR-203 and scrambled probes as controls were used.

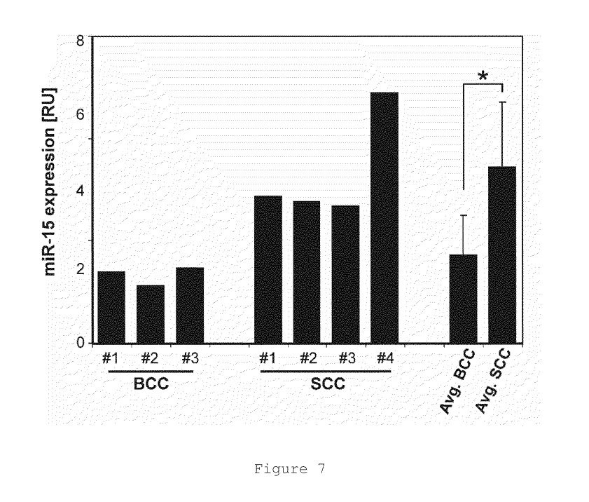

FIG. 6 shows a heatmap showing the results of unsupervised hierarchical clustering (Euclidian distance, average linkage) on a subset of 25 miRNAs that were differentially expressed (FDR<2.5%) between SCC and BCC as determined by SAM analysis. Heatmap colors represent relative miRNA expression. A median expression value equal to 1 was designated black; dark grey, increased expression; light grey, reduced expression. Note that the color scale is logarithmic (i.e. 2 means 4-fold change, 0 means no change).

FIG. 7 shows the increased expression of miR-15 in SCC P<0.05.

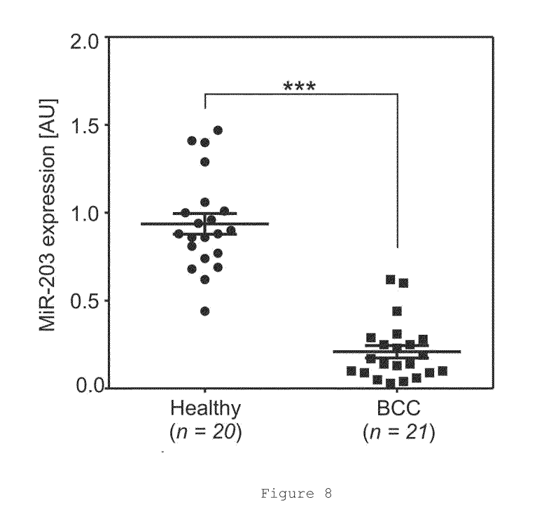

FIG. 8 shows the down-regulation of miR-203 in human BCCs. *** P<0.00001. Expression of the functionally active, mature form of miR-203 in healthy human skin (n=20) and in basal carcinoma (n=21) was measured by quantitative real-time PCR.

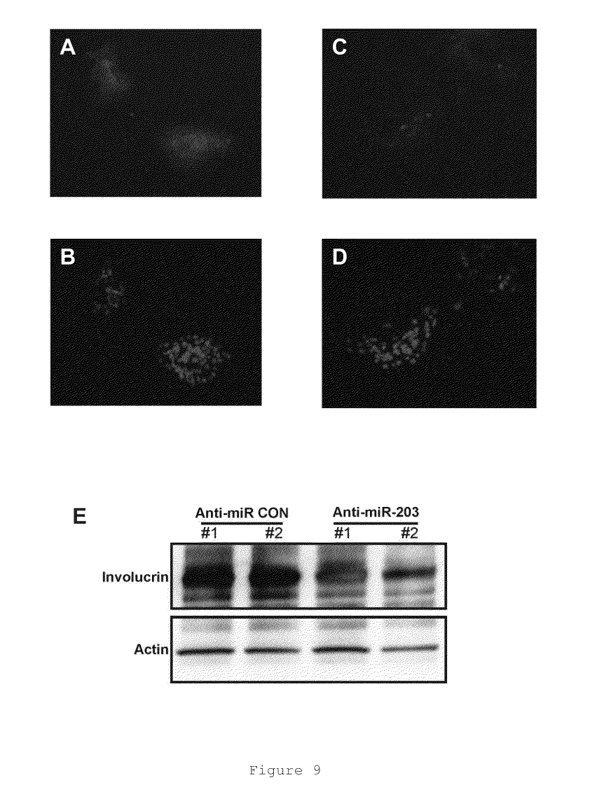

FIG. 9 shows regulation of keratinocyte differentiation by miR-203. Primary human keratinocytes were transiently transfected with (A&B) a scrambled inhibitor (anti-miR-CON) or (C&D) a specific miR-203 inhibitor (anti-miR-203) and treated with 1.5 mM calcium for 72 hours. (A&C) The expression of the keratinocyte-specific differentiation marker involucrin was visualized by immunofluorescent staining 72 hours after calcium-treatment. (B&D) Cell nuclei were visualized by DAPI. Magnification: 200×. (E) Primary human keratinocytes were transiently transfected with a specific miR-203 inhibitor (anti-miR-203) or scrambled inhibitor (anti-miR-CON) and treated with 1.5 mM calcium. The expression of involucrin was measured by Western blotting.

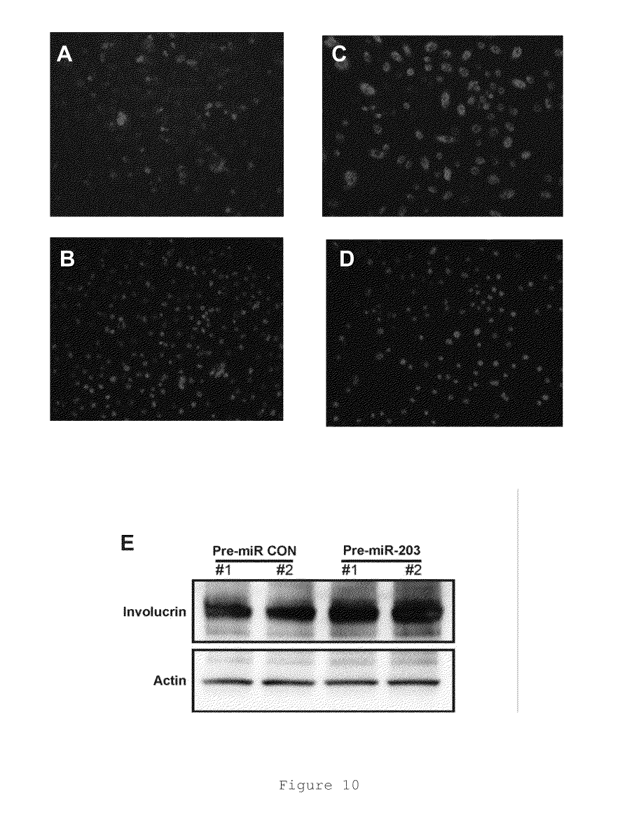

FIG. 10 shows regulation of keratinocyte differentiation by miR-203. Normal human keratinocytes cultured in low-calcium medium were transfected with (A&B) scrambled oligos as negative control (pre-miR-CON) or (C&D) a synthetic precursor molecule for miR-203 (pre-miR-203). (A&C) The expression of involucrin was visualized by immunofluorescent staining in the keratinocytes 96 hours after transfection. (B&D) Cell nuclei were visualized by DAPI. Magnification: 200×. (E) Western blotting was used to analyze the expression of involucrin in the keratinocytes 96 hours after transfection with pre-miR-203 or pre-miR-CON.

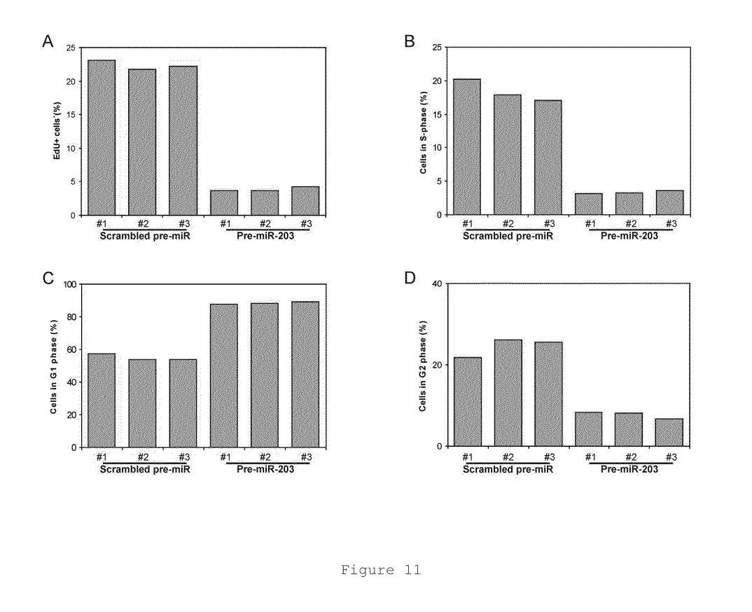

FIG. 11 shows the effect of overexpression of miR-203 on keratinocyte proliferation. Normal human keratinocytes cultured in low-calcium medium were transfected with a synthetic precursor molecule for miR-203 (pre-miR-203) or scrambled oligos as negative control (Scrambled pre-miR). Cell cycle analysis was performed by flow cytometry using EdU-assay to give (A) the percentage of cells that underwent cell division (percentage of EdU+ cells), (B) the percentage of cells in the S-phase of the cell cycle, (C) the percentage of cells in the G1-phase of the cell cycle, and (D) the percentage of cells in the G2-phase of the cell cycle.

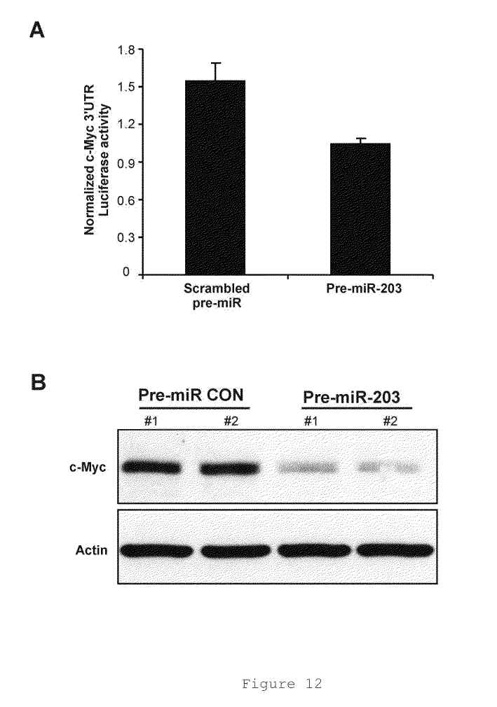

FIG. 12 shows the regulation of c-Myc oncogene by miR-203. Reporter gene analysis was carried out using c-Myc 3′ UTR constructs. (A) Cells were co-transfected with synthetic miR-203 (pre-miR-203) or scrambled miRNAs (Scrambled pre-miR) and pMIR-MYC 3′UTR reporter construct and pRenilla construct. Luciferase activity was normalized to Renilla luciferase activity. (B) Cell lysate was prepared from keratinocytes cotransfected with synthetic miR-203 (pre-miR-203) or scrambled miRNA (Scrambled pre-miR), followed by Western blot probing with the anti-myc antibody 72 hours after transfection, or with anti-actin antibody using the same blot to confirm equal loading.

Table 1 shows miRNA genes significantly up or down-regulated in SCC or BCC relative to healthy skin.

Table 2 shows miRNA genes significantly up-regulated in SCC or BCC relative to healthy skin.

Table 3 shows miRNA genes which are significantly down-regulated in either SCC or BCC relative to healthy skin.

Table 4a shows miRNA genes which are significantly up-regulated in only SCC relative to healthy skin.

Table 4b shows analysis scores for miRNA genes which are significantly up-regulated in only SCC relative to healthy skin.

Table 5 shows miRNA genes which are significantly down-regulated in only SCC relative to healthy skin.

Table 6a shows miRNA genes which are significantly up-regulated in only BCC relative to healthy skin.

Table 6b shows analysis scores for miRNA genes which are significantly up-regulated in only BCC relative to healthy skin.

Table 7 shows miRNA genes which are significantly down-regulated in only BCC relative to healthy skin.

Table 8 shows miRNA genes significantly down-regulated in both SCC and BCC relative to healthy skin.

Table 9a shows miRNA genes which are significantly down-regulated in SCC relative to healthy skin

Table 9b shows analysis scores for miRNA genes which are significantly down-regulated in SCC relative to healthy skin

Table 10a shows miRNA genes which are significantly down-regulated in BCC relative to healthy skin.

Table 10b shows analysis scores for miRNA genes which are significantly down-regulated in BCC relative to healthy skin.

Table 11a shows miRNA genes which are significantly down-regulated in SCC relative to BCC.

Table 11b shows analysis scores for miRNA genes which are significantly down-regulated in SCC relative to BCC.

Table 12a shows miRNA genes which are significantly up-regulated in SCC relative to BCC.

Table 12b shows miRNA genes which are significantly up-regulated in SCC relative to BCC.

Table 13 shows a summary of miRNA genes whose expression is altered in BCC or SCC relative to healthy skin. Bold text is used when more than one member of a microRNA family is significantly regulated. Highlighted field indicates microRNAs which are suppressed in both Squamous and Basal Cell carcinomas.

Table 14 shows the results of miRNA expression analysis in healthy individuals and SCC patients.

Table 15 shows the results of miRNA expression analysis in BCC and SCC patients.

Table 16 shows the sequences and miRBase database identifiers for the miRNAs described herein.

### Experiments

Comprehensive, genome-wide analysis of miRNA expression was performed in healthy skin (n=4) and SCC (n=4). To this end, we analyzed the expression of human miRNAs (n=365) using the Early Access TaqMan® Human MicroRNA Array v1.0. Significance analysis of microarrays (SAM) revealed that there were 62 differentially expressed miRNAs in SCC relative to healthy skin at a 2.5% false discovery rate (FDR), with a median fold change 4.3.

Most miRNAs with significantly change expression were suppressed in tumours (FIG. 1). Moreover, unsupervised hierarchical clustering based on miRNA expression clearly separated SCC tumour samples from healthy skin and provided indication that altered expression of miRNA has a role in the pathogenesis of SCC.

One of the top down-regulated miRNAs in SCC was let-7g (FIG. 2). Comparison of let-7g expression in the human SCC cell line A431 and primary keratinocytes showed that let-7g is down-regulated in A431 cells, confirming the microarray results and indicating that A431 can be used as a model for studying microRNA functions in SCC.

64 differentially expressed miRNAs in BCC relative to healthy skin were identified. Moreover, unsupervised hierarchical clustering based on miRNA expression clearly separated BCC tumor samples from healthy skin and provided indication that altered expression of miRNA has a role in the pathogenesis of BCC (FIG. 3). One of the top down-regulated miRNAs in SCC was miR-203 (FIG. 4).

In situ hybridizations were performed on samples of healthy skin, BCC, actinic keratosis and SCC using specific LNA probes for miR-203. Scrambled probes were used as controls. miR-203 was shown to be down-regulated in BCC but not in SCC (or AK) compared to healthy skin (FIG. 5).

25 miRNAs that were differentially expressed between BCC and SCC were identified. Moreover, unsupervised hierarchical clustering based on miRNA expression clearly separated BCC tumour samples from SCC (FIG. 6). One of the top miRNAs whose expression was increased in SCC relative to BCC was miR-15 (FIG. 7).

Quantitative real-time PCR using a larger number of samples (healthy, n=20; BCC, n=21) confirmed that expression of the functionally active, mature form of miR-203 is down regulated in human BCC compared to healthy human skin (FIG. 8).

Transfection of primary human keratinocytes with a specific miR-203 inibitor (anti-miR-203) decreased the expression of the keratinocyte-specific differentiation marker involucrin compared to transfection with a control scrambled inhibitor (anti-miR-203-CON) (FIG. 9). Therefore, inhibition of miR-203 in keratinocytes inhibits cell differentiation.

On the other hand, transfection of normal human keratinocytes with a synthetic precursor molecule for miR-203 (pre-miR-203) increased the expression of involucrin compared to transfection with scrambled oligos as a negative control (pre-miR-CON) (FIG. 10). Therefore, overexpression of miR-203 induces cell differentiation.

Transient overexpression of miR-203 by transfection of normal human keratinocytes with pre-miR-203 also decreased the percentage of cells that underwent cell division, i.e. the percentage of EdU+ cells (FIG. 11A), the percentage of cells in the S-phase of the cell cycle (FIG. 11B), and the percentage of cells in the G2-phase of the cell cycle (FIG. 11D) compared to cells that were transefcted with scrambled oligos (scrambled pre-miR). However, transient overexpression of miR203 increased the percentage of cells in the G1-phase compared to cells transfected with scrambled pre miR) (FIG. 11C). Therefore, over-expression of miR-203 suppresses cell proliferation by blocking the transition from G1 to the S-phase of the cell cycle.

Overexpression of miR-203 by tranfection with synthetic miR-203 (pre-miR-203) also resulted in a decrease in cMyc 3-UTR-luciferase activity following co-transfection with a pMIR-MYC 3′UTR reporter construct (FIG. 12A). This increase in cMyc expression was confirmed by western blotting (FIG. 12B). These data indicate that miR-203 negatively regulates c-Myc oncogene.

These results provide indication that miR-203 acts as a tumor suppressor gene in keratinocytes and promotes differentiation and suppresses cell proliferation through—at least partially—suppressing the c-myc oncogene. MiR-203 probably has other targets in keratinocytes including Cyclin G1, MAPK9, PKC beta 1.

### REFERENCES

Ambros, V. (2004). Nature 431(7006): 350-5.
American Cancer Society, I. (2000). “American Cancer Society. Cancer facts and figures 2000.
Bartel, D. P. (2004) Cell 116(2): 281-97.
Babak et al., RNA, 10:1813-1819 (2004)
Castoldi, M., S. Schmidt, et al. (2006) Rna 15: 15.
Chen, C. et al. (2005). Nucleic Acids Res 33(20): e179.
Cheng, A. M. et al. (2005). Nucleic Acids Res 33(4): 1290-7.
Croker, B. A. et al. (2003). Nat Immunol 4(6): 540-5.
Czech, M. P. (2006). N Engl J Med 354(11): 1194-5.
Krutzfeldt, J. et al. (2007). Nucleic Acids Res.
Krutzfeldt, J. et al. (2005). Nature 438(7068): 685-9.
Kim et al (2006 TRENDS in Genetics 22 .3 165-173
Liu et al. Proc. Nat'l Acad. Sci. USA, 101: 9740-9744 (2004)
Lowes, M. A. et al. (2007). Nature 445(7130): 866-73.
Mattes et al Am. J. Respir. Cell Mol. Biol. 36 2007, 8-12
Orom, U. A. et al. (2006). Gene 372: 137-41.
Pivarcsi A et al (2007) PNAS USA 104(48) 19055-60.
Thomson et al., Nature Methods, 1:1-7 (2004)
Tusher, V. G. et al. (2001). PNAS USA 98(9): 5116-21.
Urosevic M et al (2002) Cancer 94 (2) 477-485.
Vermeulen, A., B. Robertson, et al. (2007). RNA 13(5): 723-30.
Yi, R., D. O'Carroll, et al. (2006). Nat Genet. 38(3): 356-62.
   Tables of miRNAs

TABLE 1
miRNA genes significantly up or down-regulated
in SCC or BCC relative to healthy skin

| miRNA | miRNA | miRNA | miRNA |
|---|---|---|---|
| (hsa-miR) | (hsa-miR) | (hsa-miR) | (hsa-miR) |
| miR-203 | miR-30e-3p | miR-196b | miR-423 |
| miR-21 | miR-95 | miR-197 | miR-424 |
| miR-31 | miR-99a | miR-199a | miR-425-5p |
| miR-16 | miR-99b | miR-199b | miR-451 |
| miR-15b | miR-100 | miR-204 | miR-452 |
| miR-125a | miR-101 | miR-210 | miR-486 |
| miR-125b | miR-126 | miR-211 | miR-487b |
| miR-200c | miR-127 | miR-214 | miR-497 |
| miR-1 | miR-130a | miR-218 | miR-509 |
| miR-10a | miR-133b | miR-221 | miR-514 |
| miR-10b | miR-135b | miR-222 | miR-532 |
| miR-20b | miR-139 | miR-223 | miR-615 |
| miR-23a | miR-145 | miR-224 | miR-660 |
| miR-23b | miR-140 | miR-296 | let-7a |
| miR-24 | miR-143 | miR-324-5p | let-7b |
| miR-26a | miR-148a | miR-324-3p | let-7c |
| miR-26b | miR-148b | miR-328 | let-7d |
| miR-27a | miR-149 | miR-335 | let-7e |
| miR-28 | miR-152 | miR-345 | let-7f |
| miR-29a | miR-181d | miR-365 | let-7g |
| miR-29c | miR-182 | miR-374 | |
| miR-30a-5p | miR-191 | miR-375 | |
| miR-30a-3p | miR-193a | miR-378 | |
| miR-30b | miR-193b | miR-382 | |
| miR-30c | miR-195 | miR-383 | |
| miR-30e-5p | miR-196a | miR-411 | |

TABLE 2
miRNA genes significantly up-regulated
in SCC or BCC relative to healthy skin

miRNA (hsa-miR)
hsa-miR-21
hsa-miR-31
hsa-miR-182
hsa-miR-135b
hsa-miR-223
hsa-miR-296
hsa-miR-424
hsa-miR-514

TABLE 3
miRNA genes which are significantly down-regulated
in either SCC or BCC relative to healthy skin

| miRNA | miRNA | miRNA | miRNA |
|---|---|---|---|
| (hsa-miR) | (hsa-miR) | (hsa-miR) | (hsa-miR) |
| miR-203 | miR-99a | miR-204 | miR-487b |
| miR-16 | miR-99b | miR-210 | miR-497 |
| miR-15b | miR-100 | miR-211 | miR-509 |
| miR-125a | miR-101 | miR-214 | miR-532 |
| miR-125b | miR-126 | miR-218 | miR-615 |
| miR-200c | miR-127 | miR-221 | miR-660 |
| miR-1 | miR-130a | miR-222 | let-7a |
| miR-10a | miR-133b | miR-224 | let-7b |
| miR-10b | miR-139 | miR-296 | let-7c |
| miR-20b | miR-145 | miR-324-5p | let-7d |
| miR-23a | miR-140 | miR-324-3p | let-7e |
| miR-23b | miR-143 | miR-328 | let-7f |
| miR-24 | miR-148a | miR-335 | let-7g |
| miR-26a | miR-148b | miR-345 | |
| miR-26b | miR-149 | miR-365 | |
| miR-27a | miR-152 | miR-374 | |
| miR-28 | miR-181d | miR-375 | |
| miR-29a | miR-191 | miR-378 | |
| miR-29c | miR-193a | miR-382 | |
| miR-30a-5p | miR-193b | miR-383 | |
| miR-30a-3p | miR-195 | miR-411 | |
| miR-30b | miR-196a | miR-423 | |
| miR-30c | miR-196b | miR-425-5p | |
| miR-30e-5p | miR-197 | miR-451 | |
| miR-30e-3p | miR-199a | miR-452 | |
| miR-95 | miR-199b | miR-486 | |

TABLE 4a
miRNA genes which are significantly up-regulated
in only SCC relative to healthy skin

miRNA (hsa-miR)
hsa-miR-21
hsa-miR-31
hsa-miR-135b
hsa-miR-223

TABLE 4b

| | | | Denominator | | |
|---|---|---|---|---|---|
| Gene Name | Score(d) | Numerator(r) | (s + s0) | Fold Change | q-value(%) |
| hsa-miR-31-4373190 | 4.209754212 | 6.22549975 | 1.47882737 | 135.1658714 | 0 |
| hsa-miR-21-4373090 | 3.701842892 | 1.913958 | 0.517028425 | 3.800883272 | 0 |
| hsa-miR-135b-4373139 | 3.144949108 | 4.2760355 | 1.359651731 | 32.24687282 | 0 |
| hsa-miR-223-4373075 | 2.648122746 | 2.0317475 | 0.767240681 | 4.639191807 | 2.43902439 |

TABLE 5
miRNA genes which are significantly down-regulated
in only SCC relative to healthy skin

| miRNA | miRNA | miRNA | miRNA |
|---|---|---|---|
| (hsa-miR) | (hsa-miR) | (hsa-miR) | (hsa-miR) |
| miR-125a | miR-99b | miR-191 | miR-375 |
| miR-125b | miR-100 | miR-196b | miR-378 |
| miR-1 | miR-101 | miR-197 | miR-423 |
| miR-26a | miR-127 | miR-199a | miR-487b |
| miR-26b | miR-130a | miR-211 | miR-497 |
| miR-29a | miR-133b | miR-218 | miR-509 |
| miR-29c | miR-139 | miR-296 | miR-615 |
| miR-30a-5p | miR-145 | miR-324-5p | let-7b |
| miR-30b | miR-140 | miR-324-3p | let-7c |
| miR-30c | miR-148a | miR-328 | let-7e |
| miR-30e-5p | miR-148b | miR-345 | let-7g |
| miR-99a | miR-181d | miR-374 | |

TABLE 6a
miRNA genes which are significantly up-regulated
in only BCC relative to healthy skin
miRNA (hsa-miR)

hsa-miR-182
hsa-miR-296
hsa-miR-424
hsa-miR-514

TABLE 6b

| | | | Denominator | | |
|---|---|---|---|---|---|
| Gene Name | Score(d) | Numerator(r) | (s + s0) | Fold Change | q-value(%) |
| hsa-miR-424-4373201 | 2.536580739 | 3.718801477 | 1.466068641 | 5.372747583 | 5.989304813 |
| hsa-miR-514-4373240 | 2.391938008 | 1.163003554 | 0.4862181 | 2.950275613 | 5.989304813 |
| hsa-miR-182-4373271 | 2.180047659 | 5.190783637 | 2.381041358 | 6.297387492 | 6.586620926 |
| hsa-miR-296-4373066 | 2.040775165 | 1.224057306 | 0.599800178 | 2.235830841 | 6.586620926 |

TABLE 7
miRNA genes which are significantly down-regulated
in only BCC relative to healthy skin

| miRNA | miRNA | miRNA | miRNA |
|---|---|---|---|
| (hsa-miR) | (hsa-miR) | (hsa-miR) | (hsa-miR) |
| hsa-miR-203 | hsa-miR-28 | hsa-miR-210 | hsa-miR-425-5p |
| hsa-miR-15b | hsa-miR-95 | hsa-miR-221 | hsa-miR-452 |
| hsa-miR-200c | hsa-miR-148b | hsa-miR-222 | hsa-miR-532 |
| hsa-miR-24 | hsa-miR-193a | hsa-miR-224 | hsa-miR-660 |
| hsa-miR-27a | hsa-miR-193b | hsa-miR-382 | |

TABLE 8
miRNA genes significantly down-regulated in
both SCC and BCC relative to healthy skin

| miRNA | miRNA | miRNA | miRNA |
|---|---|---|---|
| (hsa-miR) | (hsa-miR) | (hsa-miR) | (hsa-miR) |
| hsa-miR-16 | hsa-miR-30e-3p | hsa-miR-199b | hsa-miR-451 |
| hsa-miR-1 | hsa-miR-126 | hsa-miR-204 | hsa-miR-486 |
| hsa-miR-10a | hsa-miR-143 | hsa-miR-214 | hsa-let-7a |
| hsa-miR-10b | hsa-miR-149 | hsa-miR-335 | hsa-let-7d |
| hsa-miR-20b | hsa-miR-152 | hsa-miR-365 | hsa-let-7f |
| hsa-miR-23a | hsa-miR-195 | hsa-miR-375 | |
| hsa-miR-23b | hsa-miR-196a | hsa-miR-383 | |
| hsa-miR-30a-3p | hsa-miR-199a | hsa-miR-411 | |

TABLE 9a
miRNA genes which are significantly down-
regulated in SCC relative to healthy skin

| miRNA | miRNA | miRNA |
|---|---|---|
| (hsa-miR) | (hsa-miR) | (hsa-miR) |
| miR-16 | miR-145 | miR-374 |
| miR-125a | miR-140 | miR-375 |
| miR-125b | miR-143 | miR-378 |
| miR-1 | miR-148a | miR-383 |
| miR-10a | miR-148b | miR-411 |
| miR-10b | miR-149 | miR-423 |
| miR-23a | miR-152 | miR-451 |
| miR-23b | miR-181d | miR-486 |
| miR-26a | miR-191 | miR-487b |
| miR-26b | miR-195 | miR-497 |
| miR-29a | miR-196a | miR-509 |
| miR-29c | miR-196b | miR-615 |
| miR-30a-5p | miR-197 | let-7a |
| miR-30a-3p | miR-199a | let-7b |
| miR-30b | miR-199b | let-7c |
| miR-30c | miR-204 | let-7d |
| miR-30e-3p | miR-211 | let-7e |
| miR-99a | miR-214 | let-7f |
| miR-99b | miR-218 | let-7g |
| miR-100 | miR-296 | |
| miR-101 | miR-324-5p | |
| miR-126 | miR-324-3p | |
| miR-127 | miR-328 | |
| miR-130a | miR-335 | |
| miR-133b | miR-345 | |
| miR-139 | miR-365 | |

TABLE 9b

| | | | Denominator | | |
|---|---|---|---|---|---|
| Gene Name | Score(d) | Numerator(r) | (s + s0) | Fold Change | q-value(%) |
| hsa-let-7c-4373167 | −7.864586029 | −3.16102125 | 0.401931041 | 0.112318663 | 0 |
| hsa-miR-328-4373049 | −6.548350021 | −3.730236 | 0.569645176 | 0.080450545 | 0 |
| hsa-miR-195-4373105 | −5.977354275 | −2.8690635 | 0.479988866 | 0.141564819 | 0 |
| hsa-miR-30a-3p-4373062 | −5.14880454 | −3.370167 | 0.654553299 | 0.106370071 | 0 |
| hsa-miR-29c-4373289 | −5.134786537 | −2.270283 | 0.442137757 | 0.211823817 | 0 |
| hsa-miR-143-4373134 | −4.883578805 | −3.145944 | 0.644188233 | 0.113537391 | 0 |
| hsa-miR-125b-4373148 | −4.692083491 | −2.88632775 | 0.615148421 | 0.145211838 | 0 |
| hsa-miR-375-4373027 | −4.684346477 | −3.9534375 | 0.843967781 | 0.074075896 | 0 |
| hsa-miR-497-4373222 | −4.62064819 | −2.79907025 | 0.605774371 | 0.150722557 | 0 |
| hsa-let-7g-4373163 | −4.488647134 | −1.877719 | 0.418326267 | 0.274095314 | 0 |
| hsa-miR-199b-4373100 | −4.391719145 | −3.153642 | 0.718088269 | 0.133495018 | 0 |
| hsa-miR-26a-4373070 | −4.2301475 | −2.17039975 | 0.513078977 | 0.232777625 | 0 |
| hsa-miR-199a-4378068 | −4.197085334 | −2.080725 | 0.495754752 | 0.238656173 | 0 |
| hsa-miR-30e-5p-4373058 | −4.056961865 | −2.40537 | 0.592899337 | 0.205123708 | 0 |
| hsa-miR-204-4373094 | −4.056512765 | −3.3409545 | 0.82360261 | 0.097631378 | 0 |
| hsa-miR-10a-4373153 | −3.957807362 | −2.96116225 | 0.748182511 | 0.135607772 | 0 |
| hsa-miR-383-4373018 | −3.856242828 | −3.2499455 | 0.842775117 | 0.101440439 | 0 |
| hsa-miR-126-4373269 | −3.793241239 | −2.40465175 | 0.633930615 | 0.201097431 | 0 |
| hsa-miR-99a-4373008 | −3.776293258 | −3.16842925 | 0.839031567 | 0.137112841 | 0 |
| hsa-miR-196a-4373104 | −3.683551472 | −2.11225075 | 0.573427782 | 0.237511685 | 0 |
| hsa-miR-218-4373081 | −3.682151441 | −2.73806825 | 0.743605551 | 0.171851864 | 0 |
| hsa-miR-1-4373161 | −3.665921156 | −3.32747675 | 0.907678209 | 0.089423825 | 0 |
| hsa-miR-23b-4373073 | −3.660578292 | −2.1759615 | 0.594431078 | 0.239788681 | 0 |
| hsa-miR-100-4373160 | −3.623026386 | −3.17690425 | 0.876864784 | 0.145226645 | 0 |
| hsa-miR-149-4373128 | −3.439568726 | −3.463511 | 1.006960836 | 0.124551739 | 0 |
| hsa-miR-140-4373138 | −3.388284171 | −1.66080925 | 0.490162326 | 0.316255576 | 0 |
| hsa-miR-30e-3p-4373057 | −3.317296339 | −1.96354625 | 0.59191162 | 0.27892635 | 0 |
| hsa-miR-145-4373133 | −3.224169069 | −2.5333995 | 0.785752684 | 0.188958023 | 0 |
| hsa-miR-199a-4373272 | −3.215076872 | −2.48272225 | 0.772212407 | 0.208626836 | 0 |
| hsa-miR-214-4373085 | −3.147654118 | −1.94285275 | 0.617238323 | 0.280745365 | 0 |
| hsa-miR-335-4373045 | −3.097920839 | −2.331674 | 0.752657709 | 0.215940616 | 0 |
| hsa-miR-26b-4373069 | −2.986941554 | −1.7994395 | 0.602435457 | 0.31563849 | 0 |
| hsa-miR-196b-4373103 | −2.980225292 | −2.499541 | 0.83870874 | 0.230536431 | 0 |
| hsa-miR-101-4373159 | −2.951710521 | −2.106685 | 0.713716669 | 0.268173838 | 0 |
| hsa-miR-615-4380991 | −2.891699009 | −2.336841 | 0.808120414 | 0.220626344 | 0 |
| hsa-miR-10b-4373152 | −2.770543559 | −2.15124975 | 0.776472091 | 0.26901666 | 0 |
| hsa-let-7b-4373168 | −2.657347057 | −1.61640475 | 0.608277623 | 0.350211754 | 0 |
| hsa-let-7e-4373165 | −2.635055034 | −1.97627075 | 0.749992211 | 0.272349509 | 0 |
| hsa-miR-29a-4373065 | −2.604747541 | −1.260445 | 0.483902943 | 0.417546237 | 0 |
| hsa-miR-197-4373102 | −2.592332868 | −1.44040225 | 0.555639389 | 0.389711725 | 0 |
| hsa-miR-125a-4373149 | −2.576675024 | −1.79445275 | 0.696421835 | 0.32657844 | 0 |
| hsa-miR-451-4373209 | −2.547563863 | −2.617757 | 1.027553043 | 0.144745193 | 0 |
| hsa-miR-126-4378064 | −2.475313363 | −1.9538525 | 0.789335415 | 0.303165452 | 0 |
| hsa-miR-486-4378096 | −2.470195116 | −2.58078 | 1.044767672 | 0.14704238 | 0 |
| hsa-miR-30c-4373060 | −2.417184427 | −1.5927435 | 0.658925104 | 0.369797314 | 0 |
| hsa-miR-211-4373088 | −2.404963696 | −3.970781 | 1.651077314 | 0.27541922 | 0 |
| hsa-miR-130a-4373145 | −2.238298189 | −1.33192825 | 0.595062917 | 0.439008492 | 0 |
| hsa-miR-152-4373126 | −2.169982729 | −0.9561755 | 0.440637378 | 0.518490061 | 0 |
| hsa-let-7a-4373169 | −2.163200648 | −1.5529005 | 0.717871688 | 0.386607836 | 0 |
| hsa-miR-99b-4373007 | −2.153408076 | −1.52540125 | 0.708366086 | 0.385630962 | 0 |
| hsa-miR-30b-4373290 | −2.120574194 | −1.43881275 | 0.67850149 | 0.426484624 | 0 |
| hsa-miR-133b-4373172 | −1.945959275 | −1.864587 | 0.958183979 | 0.233427687 | 0 |
| hsa-miR-487b-4378102 | −1.934307607 | −0.94720025 | 0.489684395 | 0.519309316 | 0 |
| hsa-miR-127-4373147 | −1.823766075 | −1.211194 | 0.66411697 | 0.480334265 | 0.834403081 |
| hsa-let-7d-4373166 | −1.781543543 | −1.14202625 | 0.641031904 | 0.482299854 | 0.834403081 |
| hsa-miR-378-4373024 | −1.708860701 | −1.4808445 | 0.866568293 | 0.378622586 | 1.53422502 |
| hsa-let-7f-4373164 | −1.703474992 | −0.911063 | 0.534826167 | 0.53893846 | 1.53422502 |
| hsa-miR-296-4373066 | −1.693695937 | −0.8704945 | 0.513961498 | 0.570565103 | 1.53422502 |
| hsa-miR-148a-4373130 | −1.655188822 | −1.41308875 | 0.853732657 | 0.468821251 | 1.53422502 |
| hsa-miR-324-5p-4373052 | −1.610146847 | −0.7673655 | 0.476581066 | 0.613316924 | 1.53422502 |
| hsa-miR-23a-4373074 | −1.569846207 | −1.2229465 | 0.779023126 | 0.506618243 | 1.53422502 |
| hsa-miR-423-4373015 | −1.56348159 | −0.921182 | 0.589186343 | 0.510271478 | 1.53422502 |
| hsa-miR-139-4373176 | −1.444426176 | −1.162457 | 0.804788101 | 0.485047373 | 1.53422502 |
| hsa-miR-148b-4373129 | −1.409134799 | −1.151377 | 0.817080808 | 0.561276439 | 1.53422502 |
| hsa-miR-324-3p-4373053 | −1.394316206 | −0.6213325 | 0.445618073 | 0.665785363 | 1.53422502 |
| hsa-miR-365-4373194 | −1.375146815 | −1.0541705 | 0.766587602 | 0.578136422 | 1.53422502 |
| hsa-miR-181d-4373180 | −1.368853992 | −0.58172875 | 0.424975018 | 0.685381279 | 1.53422502 |
| hsa-miR-345-4373039 | −1.353905145 | −0.77918825 | 0.575511699 | 0.541422 | 2.43902439 |
| hsa-miR-30a-5p-4373061 | −1.350243587 | −1.269987 | 0.940561401 | 0.622074273 | 2.43902439 |
| hsa-miR-191-4373109 | −1.312645271 | −0.632335 | 0.481725729 | 0.626626371 | 2.43902439 |
| hsa-miR-509-4373234 | −1.288457147 | −1.5718555 | 1.21995171 | 0.577483738 | 2.43902439 |
| hsa-miR-411-4381013 | −1.280429448 | −1.231068 | 0.961449302 | 0.657330809 | 2.43902439 |
| hsa-miR-374-4373028 | −1.280409313 | −0.585717 | 0.457445126 | 0.661134163 | 2.43902439 |
| hsa-miR-16-4373121 | −1.268219121 | −0.7340025 | 0.578766309 | 0.579026447 | 2.43902439 |

TABLE 10a
miRNA genes which are significantly down-
regulated in BCC relative to healthy skin

| miRNA | miRNA | miRNA | miRNA |
|---|---|---|---|
| (hsa-miR) | (hsa-miR) | (hsa-miR) | (hsa-miR) |
| miR-203 | miR-30e-3p | miR-210 | miR-486 |
| miR-16 | miR-95 | miR-214 | miR-532 |
| miR-15b | miR-126 | miR-221 | miR-660 |
| miR-200c | miR-143 | miR-222 | let-7a |
| miR-1 | miR-148b | miR-224 | let-7d |
| miR-10a | miR-149 | miR-335 | let-7f |
| miR-10b | miR-152 | miR-365 | |
| miR-20b | miR-193a | miR-375 | |
| miR-23a | miR-193b | miR-382 | |
| miR-23b | miR-195 | miR-383 | |
| miR-24 | miR-196a | miR-411 | |
| miR-27a | miR-199a | miR-425-5p | |
| miR-28 | miR-199b | miR-451 | |
| miR-30a-3p | miR-204 | miR-452 | |

TABLE 10b

| | | | Denominator | | |
|---|---|---|---|---|---|
| Gene ID | Score(d) | Numerator(r) | (s + s0) | Fold Change | q-value(%) |
| hsa-miR-425-5p-4380926 | −3.166028764 | −0.676353298 | 0.213628286 | 0.265594143 | 0 |
| hsa-miR-203-4373095 | −2.653364243 | −0.931827728 | 0.351187264 | 0.10929975 | 0 |
| hsa-miR-20b-4373263 | −2.010469423 | −1.057802732 | 0.526147138 | 0.282254968 | 0 |
| hsa-miR-452-4378077 | −1.995707082 | −0.586158676 | 0.293709774 | 0.335793218 | 0 |
| hsa-miR-15b-4373122 | −1.97956322 | −1.198774798 | 0.605575405 | 0.267763364 | 0 |
| hsa-miR-210-4373089 | −1.798220787 | −0.595936075 | 0.331403173 | 0.301119521 | 0 |
| hsa-miR-532-4380928 | −1.795959394 | −0.53501979 | 0.297901941 | 0.263525912 | 0 |
| hsa-miR-660-4380925 | −1.749523705 | −0.579362313 | 0.331154308 | 0.395916761 | 0 |
| hsa-miR-451-4373209 | −1.729517143 | −2.826916916 | 1.634512226 | 0.052575299 | 0 |
| hsa-miR-30a-3p-4373062 | −1.727285389 | −0.634948084 | 0.367598827 | 0.280625643 | 0 |
| hsa-miR-16-4373121 | −1.698689298 | −0.834197196 | 0.491082858 | 0.422344598 | 0 |
| hsa-miR-411-4381013 | −1.691756323 | −0.487154019 | 0.287957558 | 0.36945646 | 0 |
| hsa-miR-221-4373077 | −1.648808447 | −0.515619271 | 0.312722362 | 0.169919213 | 0 |
| hsa-miR-204-4373094 | −1.627785958 | −0.967324097 | 0.594257551 | 0.304177903 | 0 |
| hsa-miR-95-4373011 | −1.623497625 | −0.698026787 | 0.429952454 | 0.389659468 | 0 |
| hsa-miR-214-4373085 | −1.603521038 | −0.554374792 | 0.345723429 | 0.350044893 | 0 |
| hsa-miR-486-4378096 | −1.595637277 | −2.889385148 | 1.810803238 | 0.063327411 | 0 |
| hsa-miR-152-4373126 | −1.571614047 | −0.516848396 | 0.328864709 | 0.212870755 | 0 |
| hsa-miR-199a-4378068 | −1.544843176 | −0.517442314 | 0.334948118 | 0.329550675 | 0 |
| hsa-miR-365-4373194 | −1.539620533 | −0.65450951 | 0.425110925 | 0.306346112 | 0 |
| hsa-miR-375-4373027 | −1.517838151 | −0.656945098 | 0.432816304 | 0.23979268 | 0 |
| hsa-miR-199b-4373100 | −1.491647115 | −0.529128327 | 0.35472755 | 0.330711071 | 0 |
| hsa-miR-193b-4373185 | −1.477128841 | −0.513343801 | 0.347528115 | 0.230556455 | 0 |
| hsa-miR-224-4373187 | −1.412237136 | −0.579333578 | 0.410224008 | 0.423977736 | 0 |
| hsa-miR-195-4373105 | −1.407154873 | −0.499674671 | 0.355095719 | 0.335736075 | 0 |
| hsa-miR-126-4373269 | −1.390788343 | −0.530434549 | 0.381391282 | 0.356945027 | 2.237762238 |
| hsa-miR-200c-4373096 | −1.375041779 | −0.411822576 | 0.299498228 | 0.400763135 | 2.237762238 |
| hsa-miR-193a-4373107 | −1.364417473 | −0.540256652 | 0.3959614 | 0.44992577 | 2.237762238 |
| hsa-miR-24-4373072 | −1.339252627 | −0.530185845 | 0.395881878 | 0.426359734 | 2.237762238 |
| hsa-miR-27a-4373287 | −1.337011373 | −0.470116324 | 0.351617296 | 0.343143763 | 2.237762238 |
| hsa-miR-23a-4373074 | −1.334136145 | −0.520496456 | 0.390137437 | 0.256752604 | 2.237762238 |
| hsa-miR-222-4373076 | −1.317510057 | −0.399365 | 0.303121026 | 0.422563757 | 2.237762238 |
| hsa-let-7f-4373164 | −1.280366164 | −0.450962811 | 0.352213939 | 0.471732946 | 2.237762238 |
| hsa-miR-382-4373019 | −1.266969341 | −0.592300307 | 0.467493796 | 0.480626044 | 2.237762238 |
| hsa-miR-30e-3p-4373057 | −1.253253848 | −0.461411192 | 0.368170576 | 0.484756415 | 2.237762238 |
| hsa-miR-148b-4373129 | −1.246219853 | −0.452622974 | 0.363196729 | 0.450822458 | 2.237762238 |
| hsa-miR-335-4373045 | −1.228217989 | −0.566789348 | 0.461472925 | 0.473993207 | 2.237762238 |
| hsa-miR-23b-4373073 | −1.19213927 | −0.48650822 | 0.408096799 | 0.450189262 | 2.237762238 |
| hsa-let-7d-4373166 | −1.188761502 | −0.410034291 | 0.344925614 | 0.389777944 | 2.237762238 |
| hsa-miR-1-4373161 | −1.12222727 | −1.356527755 | 1.208781671 | 0.243902769 | 2.237762238 |
| hsa-miR-126-4378064 | −1.121459845 | −0.435987412 | 0.388767742 | 0.453812297 | 2.237762238 |
| hsa-let-7a-4373169 | −1.105477291 | −0.453485371 | 0.410216813 | 0.475161803 | 2.785299807 |
| hsa-miR-10a-4373153 | −1.10230045 | −0.538776125 | 0.488774295 | 0.230545369 | 2.785299807 |
| hsa-miR-143-4373134 | −1.075818134 | −0.588554689 | 0.547076379 | 0.412745778 | 2.785299807 |
| hsa-miR-10b-4373152 | −1.073274319 | −0.41930661 | 0.390679813 | 0.403828494 | 2.785299807 |
| hsa-miR-383-4373018 | −1.06071769 | −0.749951898 | 0.70702309 | 0.268770674 | 2.785299807 |
| hsa-miR-149-4373128 | −1.050476322 | −0.412953278 | 0.393110506 | 0.377281065 | 2.785299807 |
| hsa-miR-28-4373067 | −0.969148746 | −0.598175784 | 0.617217725 | 0.47956847 | 3.636363636 |
| hsa-miR-196a-4373104 | −0.769504493 | −0.392042095 | 0.509473432 | 0.438876197 | 5.788497217 |

TABLE 11a
miRNA genes which are significantly
down-regulated in SCC relative to BCC
miRNA (hsa-miR)

miR-125b
miR-26a
miR-30e-5p
miR-99a
miR-130a
miR-182
miR-218
miR-328
miR-345
miR-423
let-7c

TABLE 11b

| | | | Denominator | | |
|---|---|---|---|---|---|
| Gene ID | Score(d) | Numerator(r) | (s + s0) | Fold Change | q-value(%) |
| hsa-miR-125b-4373148 | −3.253959225 | −2.72441925 | 0.837262873 | 0.157437133 | 0 |
| hsa-let-7c-4373167 | −2.736939005 | −2.198882833 | 0.803409513 | 0.194758941 | 0 |
| hsa-miR-218-4373081 | −2.028941601 | −1.897041167 | 0.934990522 | 0.308528677 | 7.768744354 |
| hsa-miR-26a-4373070 | −1.952393546 | −1.41275175 | 0.723599887 | 0.383102746 | 7.768744354 |
| hsa-miR-130a-4373145 | −1.859476281 | −1.811339833 | 0.9741129 | 0.275802105 | 7.768744354 |
| hsa-miR-345-4373039 | −1.798547385 | −1.20922275 | 0.672332995 | 0.405689369 | 7.768744354 |
| hsa-miR-30e-5p-4373058 | −1.575868168 | −1.333369 | 0.846117097 | 0.410484742 | 15.89061345 |
| hsa-miR-423-4373015 | −1.488619482 | −1.0209375 | 0.685828388 | 0.48770026 | 15.89061345 |
| hsa-miR-182-4373271 | −1.475148921 | −2.410696667 | 1.634205627 | 0.196786457 | 15.89061345 |
| hsa-miR-99a-4373008 | −1.470178748 | −1.677466583 | 1.140994988 | 0.350954757 | 15.89061345 |

TABLE 12a
miRNA genes which are significantly
up-regulated in SCC relative to BCC

| miRNA | miRNA |
|---|---|
| (hsa-miR) | (hsa-miR) |
| hsa-miR-203 | hsa-miR-152 |
| hsa-miR-15a | hsa-miR-193a |
| hsa-miR-15b | hsa-miR-210 |
| hsa-miR-21 | hsa-miR-221 |
| hsa-miR-31 | hsa-miR-222 |
| hsa-miR-27a | hsa-miR-532 |
| hsa-miR-135b | |

TABLE 12b

| | | | Denominator | | |
|---|---|---|---|---|---|
| Gene ID | Score(d) | Numerator(r) | (s + s0) | Fold Change | q-value(%) |
| hsa-miR-31-4373190 | 2.25529691 | 4.745951167 | 2.104357589 | 31.74311621 | 0 |
| hsa-miR-152-4373126 | 2.184268782 | 1.79771725 | 0.82302932 | 3.071002974 | 0 |
| hsa-miR-15b-4373122 | 2.119629689 | 1.26929525 | 0.598828775 | 2.477264343 | 0 |
| hsa-miR-210-4373089 | 2.089221258 | 2.187363833 | 1.046975673 | 4.477180626 | 0 |
| hsa-miR-203-4373095 | 2.026863636 | 3.013483167 | 1.486771539 | 12.63315279 | 0 |
| hsa-miR-532-4380928 | 1.805036537 | 1.662951833 | 0.921284306 | 3.079567073 | 5.826558266 |
| hsa-miR-193b-4373185 | 1.62014256 | 1.734541167 | 1.07061021 | 3.565720622 | 9.988385598 |
| hsa-miR-27a-4373287 | 1.615092542 | 1.693728917 | 1.048688464 | 3.330650101 | 9.988385598 |
| hsa-miR-135b-4373139 | 1.536761138 | 2.69711525 | 1.755064716 | 9.377159108 | 9.988385598 |
| hsa-miR-21-4373090 | 1.480282002 | 1.95885 | 1.323295155 | 2.562131106 | 15.89061345 |
| hsa-miR-451-4373209 | 1.441349533 | 1.358935417 | 0.942821561 | 3.026573148 | 15.89061345 |
| hsa-miR-193a-4373107 | 1.397890321 | 1.079773833 | 0.772431011 | 2.081058846 | 15.89061345 |
| hsa-miR-15a-4373123 | 1.315233343 | 1.116879 | 0.849186957 | 2.346645096 | 22.37398374 |
| hsa-miR-222-4373076 | 1.290084396 | 1.121133833 | 0.86903914 | 2.364757527 | 22.37398374 |
| hsa-miR-486-4378096 | 1.278009602 | 1.272299417 | 0.99553197 | 2.720417097 | 22.37398374 |

TABLE 13
summary of miRNA genes whose expression is altered in BCC or SCC relative to healthy skin.

| Genes significantly | Genes significantly | Genes significantly | Genes significantly | Genes significantly | Genes significantly |
|---|---|---|---|---|---|
| down-regulated | down-regulated | up-regulated | up-regulated | down-regulated | up-regulated |
| in Squamous Cell | in Basal Cell | in Squamous Cell | in Basal Cell | in Squamous Cell | in Squamous Cell |
| Carcinoma vs. | Carcinoma vs. | Carcinoma vs. | Carcinoma vs. | Carcinoma vs. Basal | Carcinoma vs. Basal |
| Healthy skin | Healthy skin | Healthy skin | Healthy skin | Cell Carcinoma | Cell Carcinoma |
| hsa-let-7c | hsa-miR-425-5p | hsa-miR-21 | hsa-miR-424 | hsa-miR-125b-4373148 | hsa-miR-203-4373095 |
| hsa-miR-328 | hsa-miR-203 | hsa-miR-31 | hsa-miR-514 | hsa-let-7c-4373167 | hsa-miR-21-4373090 |
| hsa-miR-195 | hsa-miR-20b | hsa-miR-135b | hsa-miR-182 | hsa-miR-218-4373081 | hsa-miR-31-4373190 |
| hsa-miR-30a-3p | hsa-miR-452 | hsa-miR-223 | hsa-miR-296 | hsa-miR-26a-4373070 | hsa-miR-15b-4373122 |
| hsa-miR-29c | hsa-miR-15b | | | hsa-miR-130a-4373145 | hsa-miR-152-4373126 |
| hsa-miR-143 | hsa-miR-210 | | | hsa-miR-345-4373039 | hsa-miR-210-4373089 |
| hsa-miR-125b | hsa-miR-532 | | | hsa-miR-30e-5p-4373058 | hsa-miR-532-4380928 |
| hsa-miR-375 | hsa-miR-660 | | | hsa-miR-423-4373015 | hsa-miR-193b-4373185 |
| hsa-miR-497 | hsa-miR-451 | | | hsa-miR-182-4373271 | hsa-miR-27a-4373287 |
| hsa-let-7g | hsa-miR-30a-3p | | | hsa-miR-99a-4373008 | hsa-miR-135b-4373139 |
| hsa-miR-199b | hsa-miR-16 | | | | hsa-miR-451-4373209 |
| hsa-miR-26a | hsa-miR-411 | | | | hsa-miR-193a-4373107 |
| hsa-miR-199a | hsa-miR-221 | | | | hsa-miR-15a-4373123 |
| hsa-miR-30e-5p | hsa-miR-204 | | | | hsa-miR-222-4373076 |
| hsa-miR-204 | hsa-miR-95 | | | | hsa-miR-486-4378096 |
| hsa-miR-10a | hsa-miR-214 | | | | |
| hsa-miR-383 | hsa-miR-486 | | | | |
| hsa-miR-126 | hsa-miR-152 | | | | |
| hsa-miR-99a | hsa-miR-199a | | | | |
| hsa-miR-196a | hsa-miR-365 | | | | |
| hsa-miR-218 | hsa-miR-375 | | | | |
| hsa-miR-1 | hsa-miR-199b | | | | |
| hsa-miR-23b | hsa-miR-193b | | | | |
| hsa-miR-100 | hsa-miR-224 | | | | |
| hsa-miR-149 | hsa-miR-195 | | | | |
| hsa-miR-140 | hsa-miR-126 | | | | |
| hsa-miR-30e-3p | hsa-miR-200c | | | | |
| hsa-miR-145 | hsa-miR-193a | | | | |
| hsa-miR-199a | hsa-miR-24 | | | | |
| hsa-miR-214 | hsa-miR-27a | | | | |
| hsa-miR-335 | hsa-miR-23a | | | | |
| hsa-miR-26b | hsa-miR-222 | | | | |
| hsa-miR-196b | hsa-let-7f | | | | |
| hsa-miR-101 | hsa-miR-382 | | | | |
| hsa-miR-615 | hsa-miR-30e-3p | | | | |
| hsa-miR-10b | hsa-miR-148b | | | | |
| hsa-let-7b | hsa-miR-335 | | | | |
| hsa-let-7e | hsa-miR-23b | | | | |
| hsa-miR-29a | hsa-let-7d | | | | |
| hsa-miR-197 | hsa-miR-1 | | | | |
| hsa-miR-125a | hsa-miR-126 | | | | |
| hsa-miR-451 | hsa-let-7a | | | | |
| hsa-miR-126 | hsa-miR-10a | | | | |
| hsa-miR-486 | hsa-miR-143 | | | | |
| hsa-miR-30c | hsa-miR-10b | | | | |
| hsa-miR-211 | hsa-miR-383 | | | | |
| hsa-miR-130a | hsa-miR-149 | | | | |
| hsa-miR-152 | hsa-miR-28 | | | | |
| hsa-let-7a | hsa-miR-196a | | | | |
| hsa-miR-99b | | | | | |
| hsa-miR-30b | | | | | |
| hsa-miR-133b | | | | | |
| hsa-miR-487b | | | | | |
| hsa-miR-127 | | | | | |
| hsa-let-7d | | | | | |
| hsa-miR-378 | | | | | |
| hsa-let-7f | | | | | |
| hsa-miR-296 | | | | | |
| hsa-miR-148a | | | | | |
| hsa-miR-324-5p | | | | | |
| hsa-miR-23a | | | | | |
| hsa-miR-423 | | | | | |
| hsa-miR-139 | | | | | |
| hsa-miR-148b | | | | | |
| hsa-miR-324-3p- | | | | | |
| hsa-miR-365 | | | | | |
| hsa-miR-181d | | | | | |
| hsa-miR-345 | | | | | |
| hsa-miR-30a-5p | | | | | |
| hsa-miR-191 | | | | | |
| hsa-miR-509 | | | | | |
| hsa-miR-411 | | | | | |
| hsa-miR-374 | | | | | |
| hsa-miR-16 | | | | | |

TABLE 14

| | H1 | H02 | H08 | H03 | SCC1 | SCC2 | SSC3 | SSC4 | Avg H | Avg SCC | SD H | SD SCC |
|---|---|---|---|---|---|---|---|---|---|---|---|---|
| hsa-miR-26a | 4.75 | 4.41 | 5.33 | 6.012 | 1.91 | 1.09 | 0.916 | 0.854 | 5.1242375 | 1.192808 | 0.704582354 | 0.486535 |
| hsa-miR-203 | 3.9 | 3.99 | 5.68 | 5.254 | 5.24 | 5.68 | 0.509 | 11.11 | 4.7078138 | 5.634807 | 0.896985502 | 4.337753 |
| hsa-miR-16 | 2.52 | 3.4 | 6.45 | 3.315 | 2.92 | 1.62 | 1.954 | 2.58 | 3.9203442 | 2.269983 | 1.732799779 | 0.590038 |
| hsa-miR-200c | 2.27 | 1.89 | 1.5 | 1.7 | 1.74 | 1.2 | 0.132 | 2.627 | 1.8373568 | 1.425163 | 0.327048431 | 1.043216 |
| hsa-miR-24 | 2 | 2.3 | 2.88 | 2.126 | 1.79 | 1.36 | 0.518 | 2.564 | 2.3280314 | 1.557106 | 0.389958446 | 0.854285 |
| hsa-miR-126 | 1.75 | 1.07 | 1.95 | 1.725 | 0.75 | 0.78 | 0.223 | 0.214 | 1.6247554 | 0.49257 | 0.380166357 | 0.316555 |
| hsa-let-7b | 1.33 | 1.37 | 1.39 | 0.94 | 0.54 | 0.34 | 0.222 | 0.658 | 1.2563236 | 0.439979 | 0.212228732 | 0.195402 |
| hsa-miR-27a | 1.05 | 0.83 | 0.92 | 1.066 | 1.14 | 0.71 | 0.297 | 1.302 | 0.9682022 | 0.862374 | 0.112759266 | 0.450575 |
| hsa-miR-19b | 0.9 | 0.91 | 1.33 | 0.868 | 1.55 | 0.64 | 0.251 | 1.177 | 1.0011556 | 0.904596 | 0.217898916 | 0.575082 |
| hsa-miR-125b | 0.87 | 0.81 | 1.15 | 0.742 | 0.17 | 0.1 | 0.181 | 0.065 | 0.8924456 | 0.129594 | 0.181391671 | 0.057453 |
| hsa-miR-26b | 0.72 | 0.81 | 0.97 | 0.804 | 0.47 | 0.22 | 0.204 | 0.147 | 0.8254483 | 0.260543 | 0.106489375 | 0.145379 |
| hsa-miR-125a | 0.71 | 0.45 | 0.6 | 0.579 | 0.35 | 0.16 | 0.173 | 0.078 | 0.5863608 | 0.191493 | 0.105363244 | 0.114566 |
| hsa-miR-20a- | 0.64 | 0.52 | 0.73 | 0.52 | 1.16 | 0.39 | 0.134 | 0.751 | 0.6044524 | 0.609862 | 0.10121908 | 0.447341 |
| hsa-miR-222- | 0.59 | 0.45 | 0.54 | 0.384 | 0.53 | 0.35 | 0.593 | 0.164 | 0.4900915 | 0.408631 | 0.092249627 | 0.192511 |
| hsa-miR-30b- | 0.59 | 0.67 | 0.77 | 0.661 | 0.24 | 0.2 | 0.136 | 0.572 | 0.672143 | 0.286659 | 0.074806687 | 0.195142 |
| hsa-miR-594- | 0.55 | 0.24 | 0.59 | 0.208 | 0.63 | 0.18 | 0.23 | 0.254 | 0.3969752 | 0.325426 | 0.201472107 | 0.207232 |
| hsa-miR-92- | 0.53 | 0.47 | 0.77 | 0.511 | 0.53 | 0.28 | 0.082 | 0.459 | 0.5672892 | 0.338457 | 0.134170077 | 0.200012 |
| hsa-miR-30c | 0.51 | 0.5 | 0.67 | 0.562 | 0.23 | 0.16 | 0.089 | 0.345 | 0.5601228 | 0.207132 | 0.077430462 | 0.108928 |
| hsa-miR-195 | 0.48 | 0.4 | 0.48 | 0.486 | 0.07 | 0.07 | 0.04 | 0.086 | 0.4622869 | 0.065444 | 0.044055025 | 0.019245 |
| hsa-miR-205 | 0.43 | 0.44 | 0.35 | 0.449 | 0.85 | 0.5 | 0.03 | 0.251 | 0.4158661 | 0.40549 | 0.045228012 | 0.34942 |
| hsa-miR-223 | 0.39 | 0.5 | 0.76 | 0.509 | 3.75 | 0.86 | 1.855 | 3.554 | 0.5401077 | 2.505663 | 0.153273678 | 1.389858 |
| hsa-miR-30a-5p | 0.32 | 0.33 | 0.37 | 0.317 | 0.12 | 0.09 | 0.059 | 0.561 | 0.3345059 | 0.208088 | 0.026953351 | 0.236785 |
| hsa-miR-142-3p | 0.3 | 0.27 | 0.39 | 0.27 | 0.51 | 0.41 | 0.425 | 0.2 | 0.3072671 | 0.385508 | 0.055097546 | 0.130326 |
| hsa-miR-214 | 0.29 | 0.24 | 0.26 | 0.344 | 0.04 | 0.08 | 0.118 | 0.083 | 0.2845472 | 0.079885 | 0.04511699 | 0.033859 |
| hsa-let-7g | 0.26 | 0.23 | 0.33 | 0.254 | 0.08 | 0.07 | 0.054 | 0.087 | 0.2665743 | 0.073067 | 0.041445099 | 0.014314 |
| hsa-miR-29a | 0.25 | 0.29 | 0.45 | 0.292 | 0.11 | 0.15 | 0.175 | 0.105 | 0.3211539 | 0.134097 | 0.084613354 | 0.033846 |
| hsa-miR-100 | 0.23 | 0.22 | 0.26 | 0.23 | 0.02 | 0.03 | 0.074 | 0.008 | 0.2359621 | 0.034268 | 0.014399982 | 0.028104 |
| hsa-let-7a | 0.23 | 0.21 | 0.34 | 0.24 | 0.12 | 0.07 | 0.04 | 0.167 | 0.2567522 | 0.099262 | 0.057133756 | 0.057027 |
| hsa-let-7c | 0.22 | 0.19 | 0.24 | 0.178 | 0.03 | 0.02 | 0.026 | 0.018 | 0.2088348 | 0.023456 | 0.030281461 | 0.004203 |
| hsa-miR-11 | 0.2 | 0.23 | 0.4 | 0.237 | 0.19 | 0.14 | 0.151 | 0.187 | 0.2636465 | 0.165208 | 0.089907752 | 0.02529 |
| hsa-miR-141 | 0.19 | 0.22 | 0.14 | 0.21 | 0.18 | 0.09 | 0.009 | 0.35 | 0.1897081 | 0.155712 | 0.035568497 | 0.146248 |
| hsa-miR-152 | 0.18 | 0.13 | 0.13 | 0.169 | 0.11 | 0.07 | 0.076 | 0.063 | 0.1527784 | 0.079214 | 0.028391699 | 0.018502 |
| hsa-miR-199a | 0.18 | 0.13 | 0.14 | 0.217 | 0.03 | 0.03 | 0.054 | 0.043 | 0.1669616 | 0.039846 | 0.039438973 | 0.011392 |
| hsa-miR-126- | 0.18 | 0.12 | 0.22 | 0.196 | 0.05 | 0.05 | 0.024 | 0.02 | 0.177573 | 0.035709 | 0.041882331 | 0.01648 |
| hsa-miR-146a | 0.17 | 0.19 | 0.22 | 0.165 | 0.12 | 0.15 | 0.194 | 0.05 | 0.1858848 | 0.128732 | 0.02355032 | 0.061064 |
| hsa-miR-320 | 0.17 | 0.13 | 0.16 | 0.139 | 0.13 | 0.06 | 0.055 | 0.173 | 0.1508332 | 0.103613 | 0.018403605 | 0.056069 |
| hsa-miR-93- | 0.17 | 0.18 | 0.32 | 0.16 | 0.37 | 0.17 | 0.073 | 0.536 | 0.206939 | 0.288598 | 0.073293605 | 0.207369 |
| hsa-miR-140 | 0.14 | 0.14 | 0.23 | 0.19 | 0.06 | 0.05 | 0.071 | 0.039 | 0.1749123 | 0.055317 | 0.044478497 | 0.013497 |
| hsa-miR-30d | 0.13 | 0.14 | 0.18 | 0.111 | 0.05 | 0.04 | 0.032 | 0.269 | 0.1390663 | 0.097219 | 0.0283595 | 0.114745 |
| hsa-miR-21 | 0.13 | 0.1 | 0.18 | 0.117 | 0.54 | 0.36 | 0.411 | 0.702 | 0.132345 | 0.503028 | 0.035841063 | 0.153431 |
| hsa-miR-19a | 0.13 | 0.11 | 0.21 | 0.128 | 0.24 | 0.09 | 0.049 | 0.181 | 0.1429393 | 0.141231 | 0.047895754 | 0.085989 |
| hsa-miR-99a | 0.12 | 0.1 | 0.1 | 0.137 | 0.02 | 0.01 | 0.03 | 0.004 | 0.1156646 | 0.015859 | 0.017319151 | 0.010615 |
| hsa-miR-484 | 0.11 | 0.1 | 0.2 | 0.075 | 0.13 | 0.08 | 0.083 | 0.088 | 0.1206556 | 0.096378 | 0.053897429 | 0.025773 |
| hsa-miR-27b | 0.11 | 0.12 | 0.12 | 0.127 | 0.11 | 0.09 | 0.016 | 0.073 | 0.117458 | 0.071549 | 0.006859151 | 0.039183 |
| hsa-miR-148a | 0.11 | 0.09 | 0.09 | 0.102 | 0.05 | 0.04 | 0.011 | 0.084 | 0.0973537 | 0.045641 | 0.010243792 | 0.030232 |
| hsa-miR-146b | 0.11 | 0.07 | 0.11 | 0.085 | 0.09 | 0.09 | 0.156 | 0.05 | 0.0921916 | 0.097388 | 0.016330437 | 0.043968 |
| hsa-miR-374 | 0.1 | 0.09 | 0.16 | 0.128 | 0.1 | 0.08 | 0.073 | 0.062 | 0.1192335 | 0.078829 | 0.029328762 | 0.015559 |
| hsa-miR-196a | 0.09 | 0.07 | 0.13 | 0.084 | 0.03 | 0.01 | 0.017 | 0.026 | 0.0930437 | 0.022099 | 0.026597886 | 0.008341 |
| hsa-miR-365 | 0.09 | 0.1 | 0.14 | 0.103 | 0.05 | 0.05 | 0.022 | 0.128 | 0.1064536 | 0.061545 | 0.020534142 | 0.045843 |
| hsa-miR-331 | 0.09 | 0.09 | 0.15 | 0.105 | 0.08 | 0.06 | 0.05 | 0.113 | 0.1074516 | 0.075402 | 0.031570142 | 0.029053 |
| hsa-miR-103 | 0.09 | 0.08 | 0.09 | 0.083 | 0.08 | 0.06 | 0.054 | 0.108 | 0.0857051 | 0.076491 | 0.005452356 | 0.024521 |
| hsa-miR-149 | 0.08 | 0.06 | 0.09 | 0.069 | 0.02 | 0.01 | 0.002 | 0.005 | 0.0765543 | 0.009535 | 0.014803659 | 0.007209 |
| hsa-miR-200a | 0.08 | 0.09 | 0.07 | 0.085 | 0.13 | 0.04 | 0.005 | 0.067 | 0.0815229 | 0.060653 | 0.007409258 | 0.050422 |
| hsa-miR-15b | 0.08 | 0.1 | 0.23 | 0.138 | 0.07 | 0.07 | 0.067 | 0.122 | 0.135688 | 0.082517 | 0.068338085 | 0.026622 |
| hsa-miR-106b | 0.07 | 0.08 | 0.11 | 0.071 | 0.1 | 0.05 | 0.044 | 0.216 | 0.08377 | 0.103575 | 0.019327367 | 0.07992 |
| hsa-miR-342 | 0.07 | 0.05 | 0.06 | 0.053 | 0.04 | 0.05 | 0.073 | 0.061 | 0.0605704 | 0.056324 | 0.008152531 | 0.013984 |
| hsa-miR-29c- | 0.07 | 0.08 | 0.09 | 0.084 | 0.02 | 0.02 | 0.014 | 0.013 | 0.0807212 | 0.017099 | 0.008991856 | 0.004485 |
| hsa-miR-210 | 0.06 | 0.07 | 0.06 | 0.06 | 0.08 | 0.06 | 0.028 | 0.121 | 0.0630688 | 0.070843 | 0.006272834 | 0.039462 |
| hsa-miR-181b | 0.06 | 0.07 | 0.07 | 0.058 | 0.03 | 0.05 | 0.062 | 0.014 | 0.0633682 | 0.039233 | 0.006198417 | 0.021125 |
| hsa-miR-130a | 0.06 | 0.06 | 0.06 | 0.065 | 0.02 | 0.02 | 0.024 | 0.05 | 0.0622473 | 0.027327 | 0.00298144 | 0.01523 |
| hsa-miR-193a | 0.06 | 0.07 | 0.04 | 0.093 | 0.07 | 0.03 | 0.047 | 0.062 | 0.0645489 | 0.051538 | 0.021336478 | 0.016442 |
| hsa-miR-30e-3p | 0.06 | 0.05 | 0.07 | 0.065 | 0.01 | 0.02 | 0.012 | 0.03 | 0.0607728 | 0.016951 | 0.008693796 | 0.009007 |
| hsa-miR-211 | 0.05 | 0.04 | 0.02 | 0.028 | 0 | 0.04 | 9E−04 | 6E−04 | 0.0352824 | 0.009717 | 0.015292168 | 0.017801 |
| hsa-miR-565 | 0.05 | 0.03 | #### | 0.039 | 0.13 | 0.05 | 0.07 | 0.082 | 6189330.8 | 0.082438 | 12378661.57 | 0.034462 |
| hsa-miR-10a | 0.05 | 0.04 | 0.09 | 0.032 | 0.01 | 0.01 | 0.014 | 0.004 | 0.0525208 | 0.007122 | 0.02415109 | 0.004471 |
| hsa-miR-218 | 0.05 | 0.05 | 0.07 | 0.058 | 0 | 0.01 | 0.016 | 0.01 | 0.0577019 | 0.009916 | 0.009676266 | 0.005178 |
| hsa-miR-196b | 0.05 | 0.05 | 0.05 | 0.056 | 0.01 | 0.01 | 0.004 | 0.026 | 0.051396 | 0.011849 | 0.003056634 | 0.010009 |
| hsa-miR-25 | 0.05 | 0.04 | 0.08 | 0.053 | 0.04 | 0.03 | 0.016 | 0.076 | 0.0546019 | 0.039612 | 0.015028258 | 0.025525 |
| hsa-miR-197 | 0.05 | 0.03 | 0.04 | 0.035 | 0.01 | 0.01 | 0.01 | 0.027 | 0.0407159 | 0.015867 | 0.007395751 | 0.00734 |
| hsa-miR-186 | 0.05 | 0.05 | 0.06 | 0.043 | 0.05 | 0.03 | 0.018 | 0.051 | 0.0493316 | 0.035565 | 0.008312587 | 0.016359 |
| hsa-miR-486 | 0.05 | 0.13 | 0.41 | 0.096 | 0.05 | 0.02 | 0.011 | 0.015 | 0.1707324 | 0.025105 | 0.161509355 | 0.01851 |
| hsa-miR-23b | 0.05 | 0.06 | 0.06 | 0.05 | 0.01 | 0.01 | 0.006 | 0.021 | 0.0536569 | 0.012866 | 0.006366681 | 0.005992 |
| hsa-miR-30e-5p | 0.04 | 0.05 | 0.04 | 0.053 | 0.02 | 0.01 | 0.005 | 0.008 | 0.0478499 | 0.009815 | 0.004298949 | 0.004494 |
| hsa-miR-30a-3p | 0.04 | 0.04 | 0.05 | 0.052 | 0.01 | 0.01 | 0.002 | 0.006 | 0.0456989 | 0.004861 | 0.005162905 | 0.002007 |
| hsa-miR-221 | 0.04 | 0.02 | 0.04 | 0.022 | 0.06 | 0.03 | 0.048 | 0.013 | 0.0301647 | 0.036856 | 0.011154349 | 0.019255 |
| hsa-miR-127 | 0.04 | 0.03 | 0.03 | 0.029 | 0.01 | 0.01 | 0.026 | 0.018 | 0.0330246 | 0.015863 | 0.005068605 | 0.008144 |
| hsa-miR-532 | 0.04 | 0.03 | 0.04 | 0.025 | 0.03 | 0.02 | 0.01 | 0.028 | 0.0318134 | 0.022091 | 0.006131871 | 0.009092 |
| hsa-miR-101- | 0.04 | 0.04 | 0.05 | 0.051 | 0.01 | 0.01 | 0.005 | 0.023 | 0.0449562 | 0.012056 | 0.006679154 | 0.007602 |
| hsa-miR-660 | 0.03 | 0.03 | 0.04 | 0.035 | 0.04 | 0.02 | 0.012 | 0.047 | 0.0367417 | 0.028286 | 0.005272383 | 0.015562 |
| hsa-miR-200b | 0.03 | 0.03 | 0.02 | 0.029 | 0.08 | 0.03 | 0.002 | 0.036 | 0.0268527 | 0.037751 | 0.004902617 | 0.034259 |
| hsa-miR-151 | 0.03 | 0.03 | 0.04 | 0.029 | 0.05 | 0.02 | 0.021 | 0.03 | 0.0313743 | 0.030788 | 0.002737503 | 0.012037 |
| hsa-miR-181d | 0.03 | 0.03 | 0.03 | 0.034 | 0.02 | 0.02 | 0.031 | 0.018 | 0.0321231 | 0.022017 | 0.001937511 | 0.006246 |
| hsa-let-7f- | 0.03 | 0.02 | 0.03 | 0.04 | 0.02 | 0.01 | 0.011 | 0.021 | 0.0293816 | 0.015835 | 0.00797339 | 0.005057 |
| hsa-miR-451- | 0.03 | 0.06 | 0.23 | 0.06 | 0.03 | 0.01 | 0.006 | 0.008 | 0.09474 | 0.013713 | 0.090944711 | 0.009948 |
| hsa-miR-22 | 0.03 | 0.02 | 0.04 | 0.029 | 0.05 | 0.02 | 0.026 | 0.031 | 0.0302298 | 0.031596 | 0.008456277 | 0.009703 |
| hsa-miR-199b | 0.03 | 0.02 | 0.03 | 0.027 | 0 | 0 | 0.007 | 0.002 | 0.0262807 | 0.003508 | 0.002691929 | 0.002548 |
| hsa-miR-145 | 0.03 | 0.02 | 0.05 | 0.022 | 0 | 0.01 | 0.003 | 0.009 | 0.0312441 | 0.005904 | 0.014097731 | 0.003486 |
| hsa-miR-142-5p | 0.02 | 0.03 | 0.04 | 0.02 | 0.04 | 0.04 | 0.032 | 0.018 | 0.0278251 | 0.032531 | 0.009809706 | 0.010944 |
| hsa-miR-28 | 0.02 | 0.02 | 0.06 | 0.018 | 0.01 | 0.02 | 0.014 | 0.02 | 0.0294194 | 0.017108 | 0.017433755 | 0.004061 |
| hsa-miR-328 | 0.02 | 0.03 | 0.03 | 0.031 | 0 | 0 | 0.002 | 0.004 | 0.0264453 | 0.002128 | 0.004433753 | 0.001037 |
| hsa-miR-324-3p | 0.02 | 0.02 | 0.02 | 0.018 | 0.01 | 0.01 | 0.008 | 0.015 | 0.0176866 | 0.011776 | 0.001694444 | 0.002934 |
| hsa-miR-155 | 0.02 | 0.01 | 0.02 | 0.024 | 0.03 | 0.06 | 0.147 | 0.022 | 0.0184015 | 0.065388 | 0.005795681 | 0.057157 |
| hsa-miR-193b | 0.02 | 0.02 | 0.02 | 0.013 | 0.01 | 0.01 | 0.001 | 0.022 | 0.0162646 | 0.009792 | 0.002365448 | 0.008675 |
| hsa-miR-23a | 0.02 | 0.02 | 0.01 | 0.017 | 0.01 | 0.01 | 0.003 | 0.016 | 0.0174867 | 0.008859 | 0.003698307 | 0.005816 |
| hsa-miR-429- | 0.02 | 0.02 | 0.03 | 0.021 | 0.03 | 0.01 | 1E−03 | 0.017 | 0.0212474 | 0.015173 | 0.004124634 | 0.013326 |
| hsa-miR-182 | 0.02 | 0.01 | 0.02 | 0.023 | 0.03 | 0.02 | 0.002 | 0.032 | 0.0176997 | 0.021413 | 0.004435586 | 0.014049 |
| hsa-miR-17-5p | 0.02 | 0.02 | 0.03 | 0.017 | 0.04 | 0.02 | 0.006 | 0.055 | 0.0193441 | 0.030401 | 0.004967214 | 0.022942 |
| hsa-miR-15a | 0.02 | 0.01 | 0.02 | 0.012 | 0.03 | 0.01 | 0.014 | 0.022 | 0.013905 | 0.018007 | 0.003674598 | 0.009005 |
| hsa-miR-10b | 0.01 | 0.02 | 0.01 | 0.01 | 0.01 | 0 | 0.001 | 0.002 | 0.0135583 | 0.003647 | 0.003631982 | 0.002923 |
| hsa-miR-361 | 0.01 | 0.01 | 0.01 | 0.011 | 0.01 | 0.01 | 0.004 | 0.011 | 0.011812 | 0.00836 | 0.002273907 | 0.003808 |
| hsa-miR-99b | 0.01 | 0.01 | 0.01 | 0.012 | 0.01 | 0 | 0.005 | 0.002 | 0.0115018 | 0.004435 | 0.002488956 | 0.002217 |
| hsa-let-7d | 0.01 | 0.01 | 0.01 | 0.014 | 0 | 0.01 | 0.004 | 0.009 | 0.0117875 | 0.005685 | 0.003045928 | 0.002839 |
| hsa-miR-192 | 0.01 | 0.01 | 0.03 | 0.016 | 0.01 | 0.01 | 0.004 | 0.024 | 0.0173373 | 0.013043 | 0.007001945 | 0.008277 |
| hsa-miR-425-5p | 0.01 | 0.01 | 0.01 | 0.012 | 0.02 | 0.02 | 0.007 | 0.02 | 0.0116477 | 0.015607 | 0.00119741 | 0.00566 |
| hsa-miR-411 | 0.01 | 0.01 | 0.01 | 0.011 | 0 | 0 | 0.02 | 0.004 | 0.0106869 | 0.007025 | 0.001387851 | 0.008367 |
| hsa-miR-375 | 0.01 | 0.01 | 0.02 | 0.014 | 0 | 0 | 3E−04 | 6E−04 | 0.0134088 | 0.000993 | 0.005027878 | 0.000647 |
| hsa-miR-497 | 0.01 | 0.01 | 0.02 | 0.01 | 0 | 0 | 0.002 | 0.002 | 0.0127398 | 0.00192 | 0.003484014 | 0.00081 |
| hsa-miR-615 | 0.01 | 0 | 0.01 | 0.005 | 0 | 0 | 5E−04 | 0.003 | 0.0071296 | 0.001573 | 0.002746525 | 0.000897 |
| hsa-miR-143 | 0.01 | 0.01 | 0.02 | 0.01 | 0 | 0 | 1E−03 | 0.002 | 0.0135078 | 0.001534 | 0.005933537 | 0.000634 |
| hsa-miR-335 | 0.01 | 0.01 | 0.02 | 0.015 | 0 | 0 | 0.001 | 0.002 | 0.0131641 | 0.002843 | 0.004608353 | 0.001509 |
| hsa-miR-423- | 0.01 | 0.01 | 0.02 | 0.008 | 0.01 | 0 | 0.006 | 0.007 | 0.0119713 | 0.006109 | 0.005459078 | 0.001705 |
| hsa-miR-199a | 0.01 | 0.01 | 0.01 | 0.009 | 0 | 0 | 0.003 | 7E−04 | 0.0096259 | 0.002008 | 0.002108871 | 0.001211 |
| hsa-miR-31- | 0.01 | 0.01 | 0.01 | 0.02 | 3.59 | 0.96 | 0.079 | 1.994 | 0.0122522 | 1.656078 | 0.005572768 | 1.507327 |
| hsa-miR-130b | 0.01 | 0.01 | 0 | 0.007 | 0 | 0.01 | 0.021 | 0.022 | 0.0068805 | 0.013875 | 0.002142689 | 0.008768 |
| hsa-miR-32- | 0.01 | 0.01 | 0.02 | 0.005 | 0.01 | 0 | 0.003 | 0.021 | 0.009056 | 0.010262 | 0.005084926 | 0.008586 |
| hsa-miR-95 | 0.01 | 0.01 | 0.01 | 0.014 | 0.01 | 0.01 | 0.002 | 0.118 | 0.0109222 | 0.03417 | 0.002895313 | 0.055638 |
| hsa-miR-98 | 0.01 | 0 | 0.02 | 0.01 | 0.01 | 0.01 | 0.003 | 0.005 | 0.00921 | 0.005089 | 0.005183547 | 0.001457 |
| hsa-miR-378 | 0.01 | 0 | 0 | 0.003 | 0 | 0 | 9E−04 | 6E−04 | 0.0038949 | 0.001475 | 0.002297609 | 0.000908 |
| hsa-miR-452 | 0.01 | 0.01 | 0 | 0.006 | 0.01 | 0.01 | 0.001 | 0.009 | 0.0061211 | 0.005751 | 0.001125916 | 0.003431 |
| hsa-miR-301 | 0.01 | 0.01 | 0 | 0.006 | 0.01 | 0 | 0.013 | 0.012 | 0.0061923 | 0.010411 | 0.00112663 | 0.005123 |
| hsa-miR-345 | 0.01 | 0.01 | 0.02 | 0.01 | 0.01 | 0.01 | 0.005 | 0.004 | 0.0097899 | 0.0053 | 0.004724299 | 0.000722 |
| hsa-miR-509 | 0.01 | 0 | 0 | 0.003 | 0 | 0.01 | 3E−04 | 6E−04 | 0.003518 | 0.002032 | 0.001935535 | 0.002793 |
| hsa-miR-324-5p | 0.01 | 0.01 | 0.01 | 0.005 | 0 | 0 | 0.003 | 0.005 | 0.0054803 | 0.003361 | 0.000299114 | 0.001217 |
| hsa-miR-20b | 0.01 | 0.01 | 0.02 | 0.008 | 0.01 | 0 | 0.003 | 0.006 | 0.0092291 | 0.005258 | 0.004476479 | 0.002264 |
| hsa-miR-148b | 0.01 | 0 | 0.01 | 0.006 | 0 | 0 | 9E−04 | 0.006 | 0.0054425 | 0.003055 | 0.000673997 | 0.002329 |
| hsa-miR-383 | 0.01 | 0.01 | 0.02 | 0.004 | 0 | 0 | 3E−04 | 6E−04 | 0.007672 | 0.000778 | 0.005396995 | 0.000394 |
| hsa-miR-204 | 0.01 | 0.01 | 0 | 0.015 | 0 | 0 | 3E−04 | 6E−04 | 0.0079714 | 0.000778 | 0.004950612 | 0.000394 |
| hsa-miR-362 | 0.01 | 0 | 0 | 0.006 | 0.01 | 0.01 | 0.003 | 0.007 | 0.0048144 | 0.005619 | 0.001261768 | 0.001824 |
| hsa-miR-379 | 0.01 | 0 | 0 | 0.005 | 0 | 0 | 0.003 | 0.002 | 0.0038905 | 0.00198 | 0.001829029 | 0.000779 |
| hsa-miR-376a | 0.01 | 0 | 0 | 0.006 | 0 | 0 | 0.015 | 6E−04 | 0.0041387 | 0.004475 | 0.001876682 | 0.006922 |
| hsa-miR-132 | 0 | 0.01 | 0.01 | 0.009 | 0 | 0.01 | 0.003 | 0.032 | 0.0066528 | 0.013259 | 0.001727037 | 0.013599 |
| hsa-miR-224 | 0 | 0 | 0.01 | 0.003 | 0.02 | 0.01 | 0.002 | 0.033 | 0.0040701 | 0.015088 | 0.002048442 | 0.014608 |
| hsa-miR-1 | 0 | 0.01 | 0.02 | 0.006 | 0 | 0 | 4E−04 | 6E−04 | 0.0094986 | 0.000849 | 0.008301308 | 0.000467 |
| hsa-miR-508 | 0 | 0 | 0 | 7E−04 | 0 | 0 | 3E−04 | 6E−04 | 0.0018925 | 0.000778 | 0.001574688 | 0.000394 |
| hsa-miR-432 | 0 | 0 | 0 | 1E−03 | 0 | 0 | 0.006 | 0.004 | 0.0024986 | 0.003414 | 0.001132528 | 0.001766 |
| hsa-miR-34a | 0 | 0 | 0 | 0.001 | 0 | 0 | 0.003 | 1E−03 | 0.0022364 | 0.001708 | 0.000804242 | 0.000688 |
| hsa-miR-135b | 0 | 0 | 0 | 0.003 | 0.13 | 0.04 | 0.005 | 0.126 | 0.002331 | 0.075167 | 0.000823631 | 0.061561 |
| hsa-miR-382 | 0 | 0 | 0 | 0.005 | 0 | 0 | 0.004 | 0.004 | 0.0031508 | 0.002785 | 0.001354407 | 0.001891 |
| hsa-miR-134 | 0 | 0 | 0 | 0.005 | 0 | 0 | 0.004 | 0.001 | 0.0030923 | 0.002042 | 0.001074757 | 0.0014 |
| hsa-miR-425 | 0 | 0 | 0.01 | 0.005 | 0 | 0 | 0.003 | 0.004 | 0.0043306 | 0.003661 | 0.001879961 | 0.000687 |
| hsa-let-7e | 0 | 0 | 0 | 0.002 | 0 | 0 | 3E−04 | 6E−04 | 0.0029352 | 0.000799 | 0.001142712 | 0.000444 |
| hsa-miR-139 | 0 | 0 | 0 | 0.002 | 0 | 0 | 3E−04 | 6E−04 | 0.001734 | 0.000841 | 0.000644031 | 0.000476 |
| hsa-miR-133b | 0 | 0 | 0.01 | 0.001 | 0 | 0 | 3E−04 | 6E−04 | 0.003334 | 0.000778 | 0.003161788 | 0.000394 |
| hsa-miR-9 | 0 | 0 | 0 | 0.001 | 0 | 0 | 8E−04 | 0.012 | 0.0021813 | 0.004626 | 0.001348212 | 0.005079 |
| hsa-miR-194 | 0 | 0 | 0.01 | 0.004 | 0 | 0 | 8E−04 | 0.007 | 0.0041469 | 0.002928 | 0.002088798 | 0.00277 |
| hsa-miR-511 | 0 | 0 | 0.01 | 0.004 | 0 | 0 | 0.008 | 6E−04 | 0.0044236 | 0.003642 | 0.003909569 | 0.002928 |
| hsa-miR- | 0 | 0 | 0 | 0.002 | 0 | 0 | 0.002 | 0.001 | 0.0024432 | 0.001269 | 0.000613081 | 0.000325 |
| 487b | | | | | | | | | | | | |
| hsa-miR-650 | 0 | 0 | 0 | 0.005 | 0.01 | 0.01 | 0.002 | 0.003 | 0.0038363 | 0.005209 | 0.001425493 | 0.003831 |
| hsa-miR-489 | 0 | 0 | 0 | 0.001 | 0 | 0 | 0.002 | 6E−04 | 0.0014405 | 0.001305 | 0.000413137 | 0.000784 |
| hsa-miR-18a- | 0 | 0 | 0.01 | 0.004 | 0.01 | 0.01 | 0.001 | 0.009 | 0.00388 | 0.006003 | 0.001851069 | 0.003433 |
| hsa-miR-514 | 0 | 0 | 0 | 7E−04 | 0 | 0 | 3E−04 | 6E−04 | 0.0011961 | 0.000841 | 0.000542554 | 0.000478 |
| hsa-miR-296 | 0 | 0 | 0 | 0.002 | 0 | 0 | 9E−04 | 6E−04 | 0.0017063 | 0.000974 | 0.000242977 | 0.000352 |
| hsa-miR-107 | 0 | 0 | 0 | 0.002 | 0 | 0 | 3E−04 | 0.001 | 0.0016319 | 0.001207 | 0.000373525 | 0.000697 |
| hsa-miR-183 | 0 | 0 | 0 | 0.001 | 0.01 | 0 | 4E−04 | 0.004 | 0.0012876 | 0.003848 | 0.000291523 | 0.002781 |
| hsa-miR-485-3p | 0 | 0 | 0 | 0.002 | 0 | 0 | 0.003 | 7E−04 | 0.0016457 | 0.001624 | 0.000277811 | 0.001244 |
| hsa-miR-200a | 0 | 0 | 0 | 0.003 | 0.01 | 0 | 0.001 | 0.006 | 0.0020821 | 0.005572 | 0.00072668 | 0.005234 |
| hsa-miR-502 | 0 | 0 | 0 | 0.001 | 0 | 0 | 4E−04 | 0.001 | 0.0013136 | 0.001139 | 0.000441432 | 0.000569 |
| hsa-miR-424 | 0 | 0 | 0 | 7E−04 | 0 | 0 | 0.004 | 7E−04 | 0.0009332 | 0.00165 | 0.00047267 | 0.001341 |
| hsa-miR-501 | 0 | 0 | 0 | 7E−04 | 0 | 0 | 4E−04 | 6E−04 | 0.0020018 | 0.000894 | 0.001763867 | 0.000465 |
| hsa-miR-187 | 0 | 0 | 0 | 0.002 | 0.02 | 0.01 | 8E−04 | 0.004 | 0.0014595 | 0.007944 | 0.000697025 | 0.008374 |
| hsa-miR-340 | 0 | 0 | 0.01 | 0.004 | 0 | 0 | 0.003 | 6E−04 | 0.0034737 | 0.001606 | 0.002661528 | 0.000828 |
| hsa-miR-491 | 0 | 0 | 0.01 | 8E−04 | 0 | 0 | 9E−04 | 0.003 | 0.0022333 | 0.001602 | 0.002778041 | 0.000884 |
| hsa-miR-189 | 0 | 0 | 0 | 0.002 | 0 | 0 | 3E−04 | 0.002 | 0.0015199 | 0.001151 | 0.000368944 | 0.00064 |
| hsa-miR-550 | 0 | 0 | 0 | 0.002 | 0 | 0 | 0.002 | 0.004 | 0.0018871 | 0.001937 | 0.00116859 | 0.001394 |
| | H1 | H02 | H08 | H03 | SCC1 | SCC2 | SSC3 | SSC4 | Avg H | Avg SCC | | |

TABLE 15

| Detector | BCC1 | BCC2 | BCC3 | SCC1 | SCC2 | SCC3 | SCC4 |
|---|---|---|---|---|---|---|---|
| hsa-miR-215-4373084 | 1 | 0.110083 | 0.508182 | 4.33E+09 | 0.365301 | 0.092955 | 0.151015 |
| hsa-miR-203-4373095 | 1 | 1.015778 | 1.004846 | 11.82448 | 12.81889 | 1.149132 | 25.0875 |
| hsa-miR-223-4373075 | 1 | 0.06084 | 9.535258 | 7.038193 | 1.609319 | 3.477918 | 6.6621 |
| hsa-miR-31-4373190 | 1 | 0.073064 | 0.904946 | 45.34888 | 12.16846 | 0.996621 | 25.20362 |
| hsa-miR-16-4373121 | 1 | 1.295535 | 1.078293 | 2.141109 | 1.187491 | 1.43016 | 1.888512 |
| hsa-miR-26a-4373070 | 1 | 1 | 0.563615 | 0.523251 | 0.300326 | 0.251457 | 0.23447 |
| hsa-miR-24-4373072 | 1 | 0.603908 | 0.841604 | 1.847657 | 1.400429 | 0.53451 | 2.647517 |
| hsa-miR-200c-4373096 | 1 | 1.083401 | 0.251993 | 2.167581 | 1.503028 | 0.164117 | 3.278432 |
| hsa-miR-19b-4373098 | 1 | 0.707971 | 0.431045 | 1.587048 | 0.651949 | 0.256742 | 1.203152 |
| hsa-miR-20a-4373286 | 1 | 0.490775 | 0.319124 | 1.168982 | 0.393835 | 0.134209 | 0.755026 |
| hsa-miR-27a-4373287 | 1 | 0.305364 | 0.724884 | 2.969296 | 1.866752 | 0.777486 | 3.402526 |
| hsa-miR-205-4373093 | 1 | 1.526413 | 0.545838 | 3.423576 | 2.004964 | 0.12345 | 1.015148 |
| hsa-miR-126-4378064 | 1 | 0.312508 | 6.220864 | 2.964102 | 3.105316 | 0.884014 | 0.84821 |
| hsa-miR-594-4380958 | 1 | 0.435025 | 0.472472 | 0.766423 | 0.223173 | 0.27796 | 0.307991 |
| hsa-miR-21-4373090 | 1 | 0.129008 | 1.724912 | 2.625097 | 1.732597 | 1.991462 | 3.400335 |
| hsa-let-7b-4373168 | 1 | 1.19154 | 0.806487 | 0.946254 | 0.598526 | 0.38945 | 1.153965 |
| hsa-miR-92-4373013 | 1 | 0.61786 | 0.387788 | 0.721815 | 0.385387 | 0.111746 | 0.62527 |
| hsa-miR-222-4373076 | 1 | 0.621462 | 0.619355 | 2.275121 | 1.518898 | 2.563856 | 0.707444 |
| hsa-miR-142-3p-4373136 | 1 | 0.069425 | 3.78792 | 2.037668 | 1.662949 | 1.714066 | 0.806964 |
| hsa-miR-26b-4373069 | 1 | 0.996433 | 1.155695 | 1.327451 | 0.610966 | 0.571573 | 0.410887 |
| hsa-miR-93-4373012 | 1 | 0.408092 | 0.379509 | 1.172227 | 0.534113 | 0.228945 | 1.678417 |
| hsa-miR-125a-4373149 | 1 | 0.904828 | 0.873687 | 1.061484 | 0.497854 | 0.522875 | 0.235498 |
| hsa-miR-19a-4373099 | 1 | 0.421624 | 0.556721 | 1.838259 | 0.724748 | 0.371163 | 1.388108 |
| hsa-miR-30b-4373290 | 1 | 0.980999 | 0.535902 | 0.502816 | 0.4208 | 0.287261 | 1.207306 |
| hsa-miR-30c-4373060 | 1 | 1.272423 | 0.605412 | 0.685583 | 0.478491 | 0.262534 | 1.01744 |
| hsa-miR-191-4373109 | 1 | 1.309106 | 2.028047 | 1.962124 | 1.444144 | 1.588414 | 1.970965 |
| hsa-miR-141-4373137 | 1 | 0.584314 | 0.125239 | 1.044745 | 0.529074 | 0.051916 | 2.083669 |
| hsa-miR-125b-4373148 | 1 | 0.520947 | 27029360 | 0.180643 | 0.101197 | 0.18764 | 0.067065 |
| hsa-miR-484-4381032 | 1 | 0.161395 | 1.071621 | 1.200039 | 0.712921 | 0.735797 | 0.78589 |
| hsa-miR-565-4380942 | 1 | 0.200486 | 0.421875 | 1.475363 | 0.548977 | 0.795054 | 0.929855 |
| hsa-miR-135b-4373139 | 1 | 0.029134 | 0.287969 | 14.18888 | 4.642861 | 0.561674 | 13.9398 |
| hsa-miR-200a-4378069 | 1 | 0.535916 | 0.167469 | 1.218123 | 0.430452 | 0.051152 | 0.646883 |
| hsa-miR-320-4373055 | 1 | 1.709299 | 0.790598 | 1.848469 | 0.907413 | 0.811681 | 2.556854 |
| hsa-let-7a-4373169 | 1 | 1.310539 | 0.577987 | 1.237345 | 0.676337 | 0.39958 | 1.683283 |
| hsa-miR-146a-4373132 | 1 | 1.274113 | 2.717775 | 1.609167 | 2.063803 | 2.636763 | 0.675715 |
| hsa-miR-30a-5p-4373061 | 1 | 0.947151 | 1.063265 | 0.640804 | 0.518525 | 0.319904 | 3.064945 |
| hsa-miR-29a-4373065 | 1 | 2.984162 | 2.8894 | 1.468498 | 2.018052 | 2.384661 | 1.426746 |
| hsa-miR-27b-4373068 | 1 | 0.645802 | 0.506176 | 1.449131 | 1.209893 | 0.223372 | 0.994461 |
| hsa-miR-152-4373126 | 1 | 0.679855 | 2.387898 | 5.556005 | 3.780523 | 4.009499 | 3.310078 |
| hsa-miR-106b-4373155 | 1 | 0.357753 | 0.529393 | 0.785971 | 0.375214 | 0.327161 | 1.624115 |
| hsa-miR-374-4373028 | 1 | 0.379354 | 1.032281 | 1.549297 | 1.266349 | 1.145503 | 0.970612 |
| hsa-miR-146b-4373178 | 1 | 1.024323 | 1.928893 | 0.843087 | 0.892081 | 1.481239 | 0.474075 |
| hsa-miR-200b-4381028 | 1 | 1.351596 | 0.687208 | 6.099843 | 2.046898 | 0.156498 | 2.629259 |
| hsa-miR-103-4373158 | 1 | 0.711675 | 2.244137 | 2.440012 | 1.749206 | 1.591633 | 3.155171 |
| hsa-miR-331-4373046 | 1 | 0.429165 | 0.895466 | 1.218131 | 0.808185 | 0.727138 | 1.648964 |
| hsa-let-7g-4373163 | 1 | 0.093309 | 0.105222 | 0.079659 | 0.07188 | 0.053703 | 0.087025 |
| hsa-miR-210-4373089 | 1 | 0.472002 | 0.25916 | 2.832818 | 2.063635 | 1.009492 | 4.428352 |
| hsa-miR-15b-4373122 | 1 | 0.813455 | 1.051833 | 2.054752 | 1.976611 | 1.924531 | 3.508205 |
| hsa-miR-195-4373105 | 1 | 1.027899 | 2.960488 | 0.934407 | 0.907641 | 0.541474 | 1.175747 |
| hsa-miR-193a-4373107 | 1 | 0.478832 | 1.070468 | 2.29769 | 1.039013 | 1.620748 | 2.116206 |
| hsa-miR-140-4373138 | 1 | 0.176903 | 3.441733 | 1.036623 | 0.903812 | 1.234455 | 0.675172 |
| hsa-miR-221-4373077 | 1 | 2.027891 | 0.508182 | 15.18124 | 8.01307 | 12.94688 | 3.575805 |
| hsa-miR-126-4373269 | 1 | 0.256819 | 4.771927 | 1.997557 | 1.775833 | 0.893272 | 0.748507 |
| hsa-miR-486-4378096 | 1 | 2.440871 | 1.693058 | 9.612988 | 4.232201 | 1.99408 | 2.782634 |
| hsa-miR-148a-4373130 | 1 | 1.058636 | 0.775285 | 0.995002 | 0.713789 | 0.219085 | 1.631748 |
| hsa-miR-151-4373179 | 1 | 0.793542 | 0.996551 | 2.67005 | 1.356292 | 1.17035 | 1.646311 |
| hsa-miR-30d-4373059 | 1 | 1.280958 | 0.604081 | 0.657393 | 0.544815 | 0.437317 | 3.681488 |
| hsa-miR-186-4373112 | 1 | 0.445602 | 1.807262 | 1.644212 | 0.887036 | 0.60639 | 1.749229 |
| hsa-miR-22-4373079 | 1 | 0.090054 | 1.867484 | 2.232472 | 1.158907 | 1.297328 | 1.52673 |
| hsa-miR-365-4373194 | 1 | 1.62156 | 3.441866 | 3.559452 | 4.016133 | 1.758305 | 10.07019 |
| hsa-miR-17-5p-4373119 | 1 | 0.414697 | 0.198982 | 0.976235 | 0.364871 | 0.129406 | 1.203073 |
| hsa-miR-142-5p-4373135 | 1 | 0.448905 | 24.28984 | 11.72056 | 9.934704 | 8.629835 | 4.770657 |
| hsa-miR-342-4373040 | 1 | 0.258658 | 1.78441 | 1.028241 | 1.351149 | 1.885729 | 1.588956 |
| hsa-miR-25-4373071 | 1 | 0.225033 | 0.624322 | 0.74523 | 0.566999 | 0.323506 | 1.49172 |
| hsa-miR-214-4373085 | 1 | 0.407845 | 1.291817 | 0.368015 | 0.850718 | 1.220077 | 0.861716 |
| hsa-miR-660-4380925 | 1 | 0.472276 | 1.687986 | 2.834517 | 1.59534 | 0.943243 | 3.749972 |
| hsa-miR-429-4373203 | 1 | 0.890971 | 0.49857 | 2.458728 | 0.762737 | 0.072466 | 1.293957 |
| hsa-miR-182-4373271 | 1 | 0.316456 | 0.12683 | 0.14265 | 0.08629 | 0.009992 | 0.13976 |
| hsa-miR-196a-4373104 | 1 | 0.037158 | 0.48224 | 0.589507 | 0.251635 | 0.312553 | 0.47191 |
| hsa-miR-181b-4373116 | 1 | 0.61727 | 0.559476 | 0.280858 | 0.463063 | 0.563678 | 0.128238 |
| hsa-miR-155-4373124 | 1 | 0.106847 | 4.239555 | 1.594497 | 3.275643 | 7.739182 | 1.154309 |
| hsa-miR-532-4380928 | 1 | 0.86884 | 2.445796 | 6.064249 | 4.1583 | 1.973402 | 5.520328 |
| hsa-miR-199a-4378068 | 1 | 0.308232 | 1.004498 | 0.490903 | 0.549953 | 0.916468 | 0.725164 |
| hsa-miR-15a-4373123 | 1 | 0.959605 | 1.602202 | 4.424655 | 1.253896 | 2.106226 | 3.359618 |
| hsa-miR-451-4373209 | 1 | 0.854213 | 0.633198 | 5.100742 | 2.431029 | 1.074895 | 1.431108 |
| hsa-let-7c-4373167 | 1 | 0.589527 | 0.284955 | 0.144367 | 0.112737 | 0.134034 | 0.095625 |
| hsa-miR-100-4373160 | 1 | 0.329954 | 0.227658 | 0.135134 | 0.165194 | 0.408883 | 0.046707 |
| hsa-miR-29c-4373289 | 1 | 0.842436 | 6.228112 | 1.540478 | 1.449526 | 0.973174 | 0.918146 |
| hsa-miR-181d-4373180 | 1 | 0.335394 | 0.235457 | 0.254602 | 0.215882 | 0.380302 | 0.224058 |
| hsa-miR-187-4373111 | 1 | 0.353285 | 1.082625 | 5.773639 | 2.126344 | 0.237886 | 1.101106 |
| hsa-let-7f-4373164 | 1 | 0.406475 | 0.908432 | 1.226337 | 0.815701 | 0.703144 | 1.400864 |
| hsa-miR-149-4373128 | 1 | 0.651823 | 0.318889 | 0.536887 | 0.423518 | 0.052589 | 0.168847 |
| hsa-miR-99a-4373008 | 1 | 0.531446 | 0.332435 | 0.228915 | 0.178023 | 0.408337 | 0.056909 |
| hsa-miR-130a-4373145 | 1 | 0.762792 | 0.275599 | 0.104656 | 0.142123 | 0.162991 | 0.33982 |
| hsa-miR-30e-5p-4373058 | 1 | 0.97033 | 0.479842 | 0.517799 | 0.396796 | 0.169651 | 0.256765 |
| hsa-miR-301-4373064 | 1 | 0.500959 | 0.604078 | 1.659946 | 0.335085 | 1.471433 | 1.36934 |
| hsa-miR-28-4373067 | 1 | 0.865213 | 1.932045 | 1.454035 | 2.32724 | 1.530016 | 2.177808 |
| hsa-miR-32-4373056 | 1 | 0.654245 | 1.661777 | 3.254509 | 0.969924 | 0.827927 | 5.494937 |
| hsa-miR-200a-4373273 | 1 | 0.558671 | 0.463261 | 3.108419 | 0.534441 | 0.273743 | 1.556686 |
| hsa-miR-197-4373102 | 1 | 1.179942 | 1.811802 | 0.915373 | 1.011783 | 0.76118 | 1.948499 |
| hsa-miR-196b-4373103 | 1 | 0.010505 | 0.580908 | 0.281855 | 0.115503 | 0.098341 | 0.595301 |
| hsa-miR-324-3p-4373053 | 1 | 0.37699 | 0.49075 | 0.59016 | 0.636803 | 0.376844 | 0.713901 |
| hsa-miR-361-4373035 | 1 | 1.773407 | 1.194825 | 2.855074 | 1.699791 | 0.860385 | 2.632734 |
| hsa-miR-101-4373159 | 1 | 0.577337 | 0.346023 | 0.423697 | 0.324624 | 0.168693 | 0.807082 |
| hsa-miR-23b-4373073 | 1 | 0.653997 | 0.455353 | 0.432417 | 0.477389 | 0.238599 | 0.781576 |
| hsa-miR-192-4373108 | 1 | 3.73006 | 0.957153 | 1.449735 | 1.555709 | 0.52824 | 3.053234 |
| hsa-miR-23a-4373074 | 1 | 0.621468 | 0.863654 | 2.761689 | 1.232569 | 0.807013 | 3.918567 |
| hsa-miR-30e-3p-4373057 | 1 | 1.985552 | 1.759796 | 0.68234 | 1.040988 | 0.766375 | 1.976197 |
| hsa-miR-193b-4373185 | 1 | 0.313781 | 0.431531 | 1.751903 | 1.431433 | 0.247996 | 4.328941 |
| hsa-miR-423-4373015 | 1 | 0.49998 | 0.651605 | 0.466358 | 0.231839 | 0.327203 | 0.373706 |
| hsa-miR-20b-4373263 | 1 | 0.152599 | 0.835699 | 2.148508 | 1.082601 | 0.776029 | 1.659315 |
| hsa-miR-10b-4373152 | 1 | 0.513945 | 1.872734 | 2.144996 | 0.735119 | 0.403059 | 0.647362 |
| hsa-miR-452-4378077 | 1 | 0.064615 | 0.57602 | 2.102273 | 1.512963 | 0.272183 | 2.37259 |
| hsa-miR-183-4373114 | 1 | 0.14579 | 0.098861 | 0.287242 | 0.153698 | 0.0164 | 0.154566 |
| hsa-miR-99b-4373007 | 1 | 0.685853 | 1.25138 | 0.936933 | 0.558467 | 0.652945 | 0.219523 |
| hsa-miR-127-4373147 | 1 | 0.358125 | 2.26189 | 0.433747 | 0.851718 | 1.697498 | 1.168418 |
| hsa-miR-98-4373009 | 1 | 0.331348 | 1.101819 | 1.004956 | 0.850785 | 0.471383 | 0.824832 |
| hsa-miR-345-4373039 | 1 | 0.428252 | 0.503796 | 0.284835 | 0.295416 | 0.246805 | 0.218027 |
| hsa-miR-10a-4373153 | 1 | 0.151056 | 5.899538 | 1.54184 | 1.503071 | 3.679669 | 0.950492 |
| hsa-miR-18a-4373118 | 1 | 0.110083 | 1.232236 | 1.538479 | 2.044691 | 0.366574 | 2.519091 |
| hsa-miR-362-4378092 | 1 | 0.165874 | 1.392587 | 1.334554 | 1.782005 | 0.770763 | 1.643527 |
| hsa-miR-130b-4373144 | 1 | 0.452106 | 0.253348 | 0.168089 | 0.304862 | 0.775201 | 0.802475 |
| hsa-miR-9-4373285 | 1 | 0.071802 | 0.570341 | 0.557148 | 0.166621 | 0.096341 | 1.453875 |
| hsa-miR-335-4373045 | 1 | 0.698552 | 0.628675 | 0.536673 | 0.669225 | 0.164092 | 0.320592 |
| hsa-miR-425-4373202 | 1 | 0.315197 | 2.324836 | 1.006411 | 1.3071 | 0.829824 | 1.11408 |
| hsa-let-7d-4373166 | 1 | 1.108618 | 0.8287 | 0.896462 | 1.696993 | 0.987244 | 2.545717 |
| hsa-miR-324-5p-4373052 | 1 | 0.071797 | 0.28881 | 0.21282 | 0.156255 | 0.173524 | 0.331964 |
| hsa-miR-218-4373081 | 1 | 0.97664 | 0.755531 | 0.090429 | 0.314121 | 0.445675 | 0.273712 |
| hsa-miR-432-4373280 | 1 | 0.040336 | 2.056607 | 0.607421 | 0.240105 | 1.071054 | 0.738611 |
| hsa-miR-511-4373236 | 1 | 0.139458 | 0.988345 | 0.74353 | 1.011806 | 2.083497 | 0.163039 |
| hsa-miR-145-4373133 | 1 | 0.073288 | 3.682884 | 0.312455 | 1.081057 | 0.384752 | 1.068116 |
| hsa-miR-107-4373154 | 1 | 0.210535 | 0.508182 | 0.547433 | 0.365301 | 0.092955 | 0.295062 |
| hsa-miR-378-4373024 | 1 | 0.42056 | 0.508182 | 0.540577 | 0.664291 | 0.233287 | 0.151015 |
| hsa-miR-34a-4373278 | 1 | 0.092159 | 1.580715 | 0.431348 | 0.305823 | 0.579409 | 0.22396 |
| hsa-miR-375-4373027 | 1 | 0.556899 | 0.508182 | 0.461097 | 0.365301 | 0.092955 | 0.151015 |
| hsa-miR-199b-4373100 | 1 | 0.187569 | 0.55499 | 0.13404 | 0.270661 | 0.598895 | 0.166304 |
| hsa-miR-422b-4373016 | 1 | 0.083476 | 0.409378 | 0.464005 | 0.492204 | 0.113384 | 0.173299 |
| RNU6B-4373381 | 1 | 0.479008 | 1.143857 | 0.447528 | 1.485905 | 0.311363 | 0.173299 |
| hsa-miR-340-4373041 | 1 | 0.131621 | 0.547047 | 0.209862 | 0.233648 | 0.361033 | 0.082265 |
| hsa-miR-199a-4373272 | 1 | 0.391259 | 2.343889 | 0.382264 | 0.692501 | 0.92783 | 0.183238 |
| hsa-miR-328-4373049 | 1 | 1.059149 | 1.209453 | 0.110671 | 0.141445 | 0.1542 | 0.304769 |
| hsa-miR-615-4380991 | 1 | 0.080328 | 0.52755 | 0.251542 | 0.355053 | 0.097863 | 0.508443 |
| hsa-miR-490-4373215 | 1 | 0.064615 | 0.300024 | 0.323562 | 0.364147 | 0.071288 | 0.183238 |
| hsa-miR-501-4373226 | 1 | 0.067933 | 1.777013 | 0.305003 | 0.395552 | 0.118679 | 0.163039 |
| hsa-miR-189-4378067 | 1 | 0.133133 | 0.307797 | 0.263575 | 0.342948 | 0.076556 | 0.449778 |
| hsa-miR-452-4373281 | 1 | 0.064615 | 0.300024 | 0.291367 | 0.362151 | 0.071288 | 1.347474 |
| hsa-miR-143-4373134 | 1 | 0.083014 | 1.11533 | 0.177332 | 0.385282 | 0.163843 | 0.313042 |
| hsa-miR-376a-4373026 | 1 | 0.225551 | 1.058763 | 0.257517 | 0.342948 | 3.653461 | 0.150809 |
| hsa-miR-376b-4373196 | 1 | 0.050906 | 0.307797 | 0.257517 | 0.342948 | 0.076556 | 0.150809 |
| hsa-miR-213-4373086 | 1 | 0.019793 | 0.136551 | 0.099753 | 0.164179 | 0.094776 | 0.057805 |
| hsa-miR-7-4373014 | 1 | 0.059339 | 0.442605 | 0.299056 | 0.492204 | 0.134457 | 3.703558 |
| hsa-miR-134-4373141 | 1 | 0.187834 | 0.892494 | 0.299056 | 0.492204 | 1.192838 | 0.391234 |
| U44 | 1 | 2.393588 | 2.998053 | 9.230149 | 4.572633 | 2.793849 | 4.355897 |

TABLE 16

| miRNA | | |
|---|---|---|
| (hsa-miR) | Accession No | Sequence |
| miR-203 | MIMAT0000264 | GUGAAAUGUUUAGGACCACUAG |
| miR-21 | MIMAT0000076 | UAGCUUAUCAGACUGAUGUUGA |
| miR-31 | MIMAT0000089 | GGCAAGAUGCUGGCAUAGCUG |
| miR-16 | MIMAT0000069 | UAGCAGCACGUAAAUAUUGGCG |
| miR-15b | MIMAT0000417 | UAGCAGCACAUCAUGGUUUACA |
| miR-125a | MIMAT0000443 | UCCCUGAGACCCUUUAACCUGUG |
| miR-125b | MIMAT0000423 | UCCCUGAGACCCUAACUUGUGA |
| miR-200c | MIMAT0000617 | UAAUACUGCCGGGUAAUGAUGG |
| miR-1 | MIMAT0000416 | UGGAAUGUAAAGAAGUAUGUA |
| miR-10a | MIMAT0000253 | UACCCUGUAGAUCCGAAUUUGUG |
| miR-10b | MIMAT0000254 | UACCCUGUAGAACCGAAUUUGU |
| miR-20b | MIMAT0001413 | CAAAGUGCUCAUAGUGCAGGUAG |
| miR-23a | MIMAT0000078 | AUCACAUUGCCAGGGAUUUCC |
| miR-23b | MIMAT0000418 | AUCACAUUGCCAGGGAUUACC |
| miR-24 | MIMAT0000079 | GUGCCUACUGAGCUGAUAUCAGU |
| miR-26a | MIMAT0000082 | UUCAAGUAAUCCAGGAUAGGCU |
| miR-26b | MIMAT0000083 | UUCAAGUAAUUCAGGAUAGGU |
| miR-27a | MIMAT0000084 | UUCACAGUGGCUAAGUUCCGC |
| miR-28 | MIMAT0000085 | AAGGAGCUCACAGUCUAUUGAG |
| miR-29a | MIMAT0000086 | UAGCACCAUCUGAAAUCGGUU |
| miR-29c | MIMAT0000681 | UAGCACCAUUUGAAAUCGGUUA |
| miR-30a-5p | MIMAT0000087 | UGUAAACAUCCUCGACUGGAAG |
| miR-30a-3p | MIMAT0000088 | CUUUCAGUCGGAUGUUUGCAGC |
| miR-30b | MIMAT0000420 | UGUAAACAUCCUACACUCAGCU |
| miR-30c | MIMAT0000244 | UGUAAACAUCCUACACUCUCAGC |
| miR-30e-5p | MIMAT0000692 | UGUAAACAUCCUUGACUGGA |
| miR-30e-3p | MIMAT0000693 | CUUUCAGUCGGAUGUUUACAGC |
| miR-95 | MIMAT0000094 | UUCAACGGGUAUUUAUUGAGCA |
| miR-99a | MIMAT0000097 | AACCCGUAGAUCCGAUCUUGUG |
| miR-99b | MIMAT0000689 | CACCCGUAGAACCGACCUUGCG |
| miR-100 | MIMAT0000098 | AACCCGUAGAUCCGAACUUGUG |
| miR-101 | MIMAT0000099 | UACAGUACUGUGAUAACUGAAG |
| miR-126 | MIMAT0000445 | UCGUACCGUGAGUAAUAAUGCG |
| miR-127 | MIMAT0000446 | UCGGAUCCGUCUGAGCUUGGCU |
| miR-130a | MIMAT0000425 | CAGUGCAAUGUUAAAAGGGCAU |
| miR-133b | MIMAT0000770 | UUGGUCCCCUUCAACCAGCUA |
| miR-135b | MIMAT0000758 | UAUGGCUUUUCAUUCCUAUGUGA |
| miR-139 | MIMAT0000250 | UCUACAGUGCACGUGUCU |
| miR-145 | MIMAT0000437 | GUCCAGUUUUCCCAGGAAUCCCU |
| miR-133b | MIMAT0000770 | UUGGUCCCCUUCAACCAGCUA |
| miR-140 | MIMAT0000431 | AGUGGUUUUACCCUAUGGUAG |
| miR-143 | MIMAT0000435 | UGAGAUGAAGCACUGUAGCUCA |
| miR-148a | MIMAT0000243 | UCAGUGCACUACAGAACUUUGU |
| miR-148b | MIMAT0000759 | UCAGUGCAUCACAGAACUUUGU |
| miR-149 | MIMAT0000450 | UCUGGCUCCGUGUCUUCACUCCC |
| miR-152 | MIMAT0000438 | UCAGUGCAUGACAGAACUUGG |
| miR-181d | MIMAT0002821 | AACAUUCAUUGUUGUCGGUGGGU |
| miR-182 | MIMAT0000259 | UUUGGCAAUGGUAGAACUCACACU |
| miR-191 | MIMAT0000440 | CAACGGAAUCCCAAAAGCAGCUG |
| miR-193a | MIMAT0000459 | AACUGGCCUACAAAGUCCCAG |
| miR-193b | MIMAT0002819 | AACUGGCCCUCAAAGUCCCGCU |
| miR-195 | MIMAT0000461 | UAGCAGCACAGAAAUAUUGGC |
| miR-196a | MIMAT0000226 | UAGGUAGUUUCAUGUUGUUGG |
| miR-196b | MIMAT0001080 | UAGGUAGUUUCCUGUUGUUGGG |
| miR-197 | MIMAT0000227 | UUCACCACCUUCUCCACCCAGC |
| miR-199a | MIMAT0000231 | CCCAGUGUUCAGACUACCUGUUC |
| miR-199b | MIMAT0000263 | CCCAGUGUUUAGACUAUCUGUUC |
| miR-204 | MIMAT0000265 | UUCCCUUUGUCAUCCUAUGCCU |
| miR-210 | MIMAT0000267 | CUGUGCGUGUGACAGCGGCUGA |
| miR-211 | MIMAT0000268 | UUCCCUUUGUCAUCCUUCGCCU |
| miR-214 | MIMAT0000271 | ACAGCAGGCACAGACAGGCAGU |
| miR-218 | MIMAT0000275 | UUGUGCUUGAUCUAACCAUGU |
| miR-221 | MIMAT0000278 | AGCUACAUUGUCUGCUGGGUUUC |
| miR-222 | MIMAT0000279 | AGCUACAUCUGGCUACUGGGUCUC |
| miR-223 | MIMAT0000280 | UGUCAGUUUGUCAAAUACCCC |
| miR-224 | MIMAT0000281 | CAAGUCACUAGUGGUUCCGUU |
| miR-296 | MIMAT0000690 | AGGGCCCCCCCUCAAUCCUGU |
| miR-324-5p | MIMAT0000761 | CGCAUCCCCUAGGGCAUUGGUGU |
| miR-324-3p | MIMAT0000762 | ACUGCCCCAGGUGCUGCUGG |
| miR-328 | MIMAT0000752 | CUGGCCCUCUCUGCCCUUCCGU |
| miR-335 | MIMAT0000765 | UCAAGAGCAAUAACGAAAAAUGU |
| miR-345 | MIMAT0000772 | GCUGACUCCUAGUCCAGGGCUC |
| miR-365 | MIMAT0000710 | UAAUGCCCCUAAAAAUCCUUAU |
| miR-374 | MIMAT0000727 | UUAUAAUACAACCUGAUAAGUG |
| miR-375 | MIMAT0000728 | UUUGUUCGUUCGGCUCGCGUGA |
| miR-378 | MIMAT0000731 | CUCCUGACUCCAGGUCCUGUGU |
| miR-382 | MIMAT0000737 | GAAGUUGUUCGUGGUGGAUUCG |
| miR-383 | MIMAT0000738 | AGAUCAGAAGGUGAUUGUGGCU |
| miR-411 | MIMAT0003329 | UAGUAGACCGUAUAGCGUACG |
| miR-423 | MIMAT0001340 | AGCUCGGUCUGAGGCCCCUCAG |
| miR-424 | MIMAT0001341 | CAGCAGCAAUUCAUGUUUUGAA |
| miR-425-5p | MIMAT0003393 | AAUGACACGAUCACUCCCGUUGA |
| miR-451 | MIMAT0001631 | AAACCGUUACCAUUACUGAGUUU |
| miR-452 | MIMAT0001635 | AACUGUUUGCAGAGGAAACUGA |
| miR-486 | MIMAT0002177 | UCCUGUACUGAGCUGCCCCGAG |
| miR-487b | MIMAT0003180 | AAUCGUACAGGGUCAUCCACUU |
| miR-497 | MIMAT0002820 | CAGCAGCACACUGUGGUUUGU |
| miR-509 | MIMAT0002881 | UGAUUGGUACGUCUGUGGGUAGA |
| miR-514 | MIMAT0002883 | AUUGACACUUCUGUGAGUAG |
| miR-532 | MIMAT0002888 | CAUGCCUUGAGUGUAGGACCGU |
| miR-615 | MIMAT0003283 | UCCGAGCCUGGGUCUCCCUCU |
| miR-660 | MIMAT0003338 | UACCCAUUGCAUAUCGGAGUUG |
| let-7a | MIMAT0000062 | UGAGGUAGUAGGUUGUAUAGUU |
| let-7b | MIMAT0000063 | UGAGGUAGUAGGUUGUGUGGUU |
| let-7c | MIMAT0000064 | UGAGGUAGUAGGUUGUAUGGUU |
| let-7d | MIMAT0000065 | AGAGGUAGUAGGUUGCAUAGU |
| Let-7e | MIMAT0000066 | UGAGGUAGGAGGUUGUAUAGU |
| let-7f | MIMAT0000067 | UGAGGUAGUAGAUUGUAUAGUU |
| let-7g | MIMAT0000414 | UGAGGUAGUAGUUUGUACAGUU |

1. A method of assessing non-melanoma skin cancer in an individual comprising;
determining the expression of one or more of the miRNAs selected from the group consisting of miR-203, miR-21, miR-31, miR-16, miR-15b, miR-125a, miR-125b, miR-200c, miR-1, miR-10a, miR-10b, miR-20b, miR-23a, miR-23b, miR-24, miR-26a, miR-26b, miR-27a, miR-28, miR-29a, miR-29c, miR-30a-5p, miR-30a-3p, miR-30b, miR-30c, miR-30e-5p, miR-30e-3p, miR-95, miR-99a, miR-99b, miR-100, miR-101, miR-126, miR-127, miR-130a, miR-133b, miR-135b, miR-139, miR-145, miR-140, miR-143, miR-148a, miR-148b, miR-149, miR-152, miR-181d, miR-182, miR-191, miR-193a, miR-193b, miR-195, miR-196a, miR-196b, miR-197, miR-199a, miR-199b, miR-204, miR-210, miR-211, miR-214, miR-218, miR-221, miR-222, miR-223, miR-224, miR-296, miR-324-5p, miR-324-3p, miR-328, miR-335, miR-345, miR-365, miR-374, miR-375, miR-378, miR-382, miR-383, miR-411, miR-423, miR-424, miR-425-5p, miR-451, miR-452, miR-486, miR-487b, miR-497, miR-509, miR-514, miR-532, miR-615, miR-660, let-7a, let-7b, let-7c, let-7d, let-7e, let-7f and let-7g in a sample obtained from the individual, wherein a change in expression of the one or more miRNAs in the sample relative to controls is indicative that the individual has a non-melanoma skin cancer.

2. (canceled)

3. A method according to claim 1 comprising determining the expression of one or more of the miRNAs selected from the group consisting of miR-203, miR-16, miR-15b, miR-125a, miR-125b, miR-200c, miR-1, miR-10a, miR-10b, miR-20b, miR-23a, miR-23b, miR-24, miR-26a, miR-26b, miR-27a, miR-28, miR-29a, miR-29c, miR-30a-5p, miR-30a-3p, miR-30b, miR-30c, miR-30e-5p, miR-30e-3p, miR-95, miR-99a, miR-99b, miR-100, miR-101, miR-126, miR-127, miR-130a, miR-133b, miR-139, miR-145, miR-140, miR-143, miR-148a, miR-148b, miR-149, miR-152, miR-181d, miR-191, miR-193a, miR-193b, miR-195, miR-196a, miR-196b, miR-197, miR-199a, miR-199b, miR-204, miR-210, miR-211, miR-214, miR-218, miR-221, miR-222, miR-224, miR-296, miR-324-5p, miR-324-3p, miR-328, miR-335, miR-345, miR-365, miR-374, miR-375, miR-378, miR-382, miR-383, miR-411, miR-423, miR-425-5p, miR-451, miR-452, miR-486, miR-487b, miR-497, miR-509, miR-532, miR-615, miR-660, let-7a, let-7b, let-7c, let-7d, let-7e, let-7f and let-7g in the sample, wherein a decrease in the expression of one or more of these miRNAs in the sample relative to controls is indicative that the individual has a non-melanoma skin cancer.

4. (canceled)

5. A method according to claim 1 comprising;
determining the expression of one or more of the miRNAs selected from the group consisting of hsa-miR-21, hsa-miR-31, hsa-miR-182, hsa-miR-135b, hsa-miR-223, hsa-miR-296, hsa-miR-424 and hsa-miR-514 in the sample, wherein an increase in the expression of one or more of these miRNAs in the sample relative to controls is indicative that the individual has a non-melanoma skin cancer.

6. (canceled)

7. A method according to claim 1 wherein the non-melanoma skin cancer is squamous cell carcinoma or basal cell carcinoma.

8-20. (canceled)

21. A method of treatment of a non-melanoma skin cancer in an individual comprising;
increasing or reducing the expression or activity of one or more miRNAs selected from the group consisting of miR-203, miR-21, miR-31, miR-16, miR-15b, miR-125a, miR-125b, miR-200c, miR-1, miR-10a, miR-10b, miR-20b, miR-23a, miR-23b, miR-24, miR-26a, miR-26b, miR-27a, miR-28, miR-29a, miR-29c, miR-30a-5p, miR-30a-3p, miR-30b, miR-30c, miR-30e-5p, miR-30e-3p, miR-95, miR-99a, miR-99b, miR-100, miR-101, miR-126, miR-127, miR-130a, miR-133b, miR-135b, miR-139, miR-145, miR-140, miR-143, miR-148a, miR-148b, miR-149, miR-152, miR-181d, miR-182, miR-191, miR-193a, miR-193b, miR-195, miR-196a, miR-196b, miR-197, miR-199a, miR-199b, miR-204, miR-210, miR-211, miR-214, miR-218, miR-221, miR-222, miR-223, miR-224, miR-296, miR-324-5p, miR-324-3p, miR-328, miR-335, miR-345, miR-365, miR-374, miR-375, miR-378, miR-382, miR-383, miR-411, miR-423, miR-424, miR-425-5p, miR-451, miR-452, miR-486, miR-487b, miR-497, miR-509, miR-514, miR-532, miR-615, miR-660, let-7a, let-7b, let-7c, let-7d, let-7e, let-7f and let-7g in skin cells of the individual.

22. A method according to claim 21 comprising;
increasing the expression or activity of one or more target miRNAs selected from the group consisting of miR-203, miR-16, miR-15b, miR-125a, miR-125b, miR-200c, miR-1, miR-10a, miR-10b, miR-20b, miR-23a, miR-23b, miR-24, miR-26a, miR-26b, miR-27a, miR-28, miR-29a, miR-29c, miR-30a-5p, miR-30a-3p, miR-30b, miR-30c, miR-30e-5p, miR-30e-3p, miR-95, miR-99a, miR-99b, miR-100, miR-101, miR-126, miR-127, miR-130a, miR-133b, miR-139, miR-145, miR-140, miR-143, miR-148a, miR-148b, miR-149, miR-152, miR-181d, miR-191, miR-193a, miR-193b, miR-195, miR-196a, miR-196b, miR-197, miR-199a, miR-199b, miR-204, miR-210, miR-211, miR-214, miR-218, miR-221, miR-222, miR-224, miR-296, miR-324-5p, miR-324-3p, miR-328, miR-335, miR-345, miR-365, miR-374, miR-375, miR-378, miR-382, miR-383, miR-411, miR-423, miR-425-5p, miR-451, miR-452, miR-486, miR-487b, miR-497, miR-509, miR-532, miR-615, miR-660, let-7a, let-7b, let-7c, let-7d, let-7e, let-7f and let-7g in the skin cells.

23. A method according to claim 21 comprising;
reducing the expression or activity of one or more target miRNAs selected from the group consisting of hsa-miR-21, hsa-miR-31, hsa-miR-182, hsa-miR-135b, hsa-miR-223, hsa-miR-296, hsa-miR-424 and hsa-miR-514 in the cells.

24. A method according to claim 21 wherein the skin cancer is squamous cell carcinoma or basal cell carcinoma.

25. A method according to claim 23 wherein skin cancer is squamous cell carcinoma and the method comprises:
reducing the expression or activity of one or more target miRNAs selected from the group consisting of hsa-miR-21, hsa-miR-31, hsa-miR-135b and hsa-miR-223 in the cells.

26. (canceled)

27. A method according to claim 21 wherein skin cancer is basal cell carcinoma and the method comprises:
reducing the expression or activity of one or more target miRNAs selected from the group consisting of hsa-miR-424, hsa-miR-514, hsa-miR-182 and hsa-miR-296 in the cells.

28. (canceled)

29. A method according to claim 23 wherein the expression or activity of one or more target miRNAs is reduced by administering a therapeutically effective amount of an miRNA inhibitor to the individual.

30. A method according to claim 29 wherein the miRNA inhibitor is a sense or anti-sense oligonucleotide which inhibits the activity or production of the target miRNA or increase its rate of depletion

31. A method according to claim 21, wherein the skin cancer is squamous cell carcinoma and the method comprises increasing the amount or activity of one or more target miRNAs selected from the group consisting of miR-16, miR-125a, miR-125b, miR-1, miR-10a, miR-10b, miR-23a, miR-23b, miR-26a, miR-26b, miR-29a, miR-29c, miR-30a-5p, miR-30a-3p, miR-30b, miR-30c, miR-30e-3p, miR-99a, miR-99b, miR-100, miR-101, miR-126, miR-127, miR-130a, miR-133b, miR-139, miR-145, miR-140, miR-143, miR-148a, miR-148b, miR-149, miR-152, miR-181d, miR-191, miR-195, miR-196a, miR-196b, miR-197, miR-199a, miR-199b, miR-204, miR-211, miR-214, miR-218, miR-296, miR-324-5p, miR-324-3p, miR-328, miR-335, miR-345, miR-365, miR-374, miR-375, miR-378, miR-383, miR-411, miR-423, miR-451, miR-452, miR-486, miR-487b, miR-497, miR-509, miR-615, let-7a, let-7b, let-7c, let-7d, let-7e, let-7f and let-7g in the cells.

32. A method according to claim 21, wherein the skin cancer is basal cell carcinoma and the method comprises comprising increasing the amount or activity of one or more target miRNAs selected from the group consisting of miR-203, miR-16, miR-15b, miR-200c, miR-1, miR-10a, miR-10b, miR-20b, miR-23a, miR-23b, miR-24, miR-27a, miR-28, miR-30a-3p, miR-30e-3p, miR-95, miR-126, miR-143, miR-148b, miR-149, miR-152, miR-193a, miR-193b, miR-195, miR-196a, miR-199a, miR-199b, miR-204, miR-210, miR-214, miR-221, miR-222, miR-224, miR-335, miR-365, miR-375, miR-382, miR-383, miR-411, miR-425-5p, miR-451, miR-452, miR-486, miR-532, miR-660, let-7a, let-7d, and let-7f in the cells.

33. A method according to claim 22 wherein the expression or activity of the target miRNA is increased by administering to an individual in need thereof a therapeutically effective amount of;
(i) the target miRNA or a precursor thereof,
(ii) a nucleic acid encoding the target miRNA or a precursor thereof,
(iii) an analogue, derivative or modified form of the target miRNA which retains activity.

34-38. (canceled)

39. A method of screening for a compound useful in the treatment of skin cancer comprising;
contacting a cell with a test compound and;
determining the expression of one or more miRNAs selected from the group consisting of miR-203, miR-21, miR-31, miR-16, miR-15b, miR-125a, miR-125b, miR-200c, miR-1, miR-10a, miR-10b, miR-20b, miR-23a, miR-23b, miR-24, miR-26a, miR-26b, miR-27a, miR-28, miR-29a, miR-29c, miR-30a-5p, miR-30a-3p, miR-30b, miR-30c, miR-30e-5p, miR-30e-3p, miR-95, miR-99a, miR-99b, miR-100, miR-101, miR-126, miR-127, miR-130a, miR-133b, miR-135b, miR-139, miR-145, miR-140, miR-143, miR-148a, miR-148b, miR-149, miR-152, miR-181d, miR-182, miR-191, miR-193a, miR-193b, miR-195, miR-196a, miR-196b, miR-197, miR-199a, miR-199b, miR-204, miR-210, miR-211, miR-214, miR-218, miR-221, miR-222, miR-223, miR-224, miR-296, miR-324-5p, miR-324-3p, miR-328, miR-335, miR-345, miR-365, miR-374, miR-375, miR-378, miR-382, miR-383, miR-411, miR-423, miR-424, miR-425-5p, miR-451, miR-452, miR-486, miR-487b, miR-497, miR-509, miR-514, miR-532, miR-615, miR-660, let-7a, let-7b, let-7c, let-7d, let-7e, let-7f and let-7g relative to controls,
wherein an increase or decrease in expression in the presence of the test compound is indicative that the compound is useful in the treatment of an skin cancer.

40. A method according to claim 39 comprising
determining the expression of one or more miRNAs selected from the group consisting of hsa-miR-21, hsa-miR-31, hsa-miR-182, hsa-miR-135b, hsa-miR-223, hsa-miR-296, hsa-miR-424 and hsa-miR-514 in the cell,
wherein a decrease in expression in the presence of the test compound relative to its absence is indicative that the compound is useful in the treatment of an skin cancer.

41. A method according to claim 39 comprising determining the expression of one or more miRNAs selected from the group consisting of miR-203, miR-16, miR-15b, miR-125a, miR-125b, miR-200c, miR-1, miR-10a, miR-10b, miR-20b, miR-23a, miR-23b, miR-24, miR-26a, miR-26b, miR-27a, miR-28, miR-29a, miR-29c, miR-30a-5p, miR-30a-3p, miR-30b, miR-30c, miR-30e-5p, miR-30e-3p, miR-95, miR-99a, miR-99b, miR-100, miR-101, miR-126, miR-127, miR-130a, miR-133b, miR-139, miR-145, miR-140, miR-143, miR-148a, miR-148b, miR-149, miR-152, miR-181d, miR-191, miR-193a, miR-193b, miR-195, miR-196a, miR-196b, miR-197, miR-199a, miR-199b, miR-204, miR-210, miR-211, miR-214, miR-218, miR-221, miR-222, miR-224, miR-296, miR-324-5p, miR-324-3p, miR-328, miR-335, miR-345, miR-365, miR-374, miR-375, miR-378, miR-382, miR-383, miR-411, miR-423, miR-425-5p, miR-451, miR-452, miR-486, miR-487b, miR-497, miR-509, miR-532, miR-615, miR-660, let-7a, let-7b, let-7c, let-7d, let-7e, let-7f and let-7g,
wherein an increase in expression of the one or more miRNAs in the presence of the test compound relative to its absence is indicative that the compound is useful in the treatment of an skin cancer.

42. A method according to claim 39 wherein the skin cancer is squamous cell carcinoma or basal cell carcinoma.

43-49. (canceled)

50. A method according to claim 39 further comprising determining the ability of said test compound to ameliorate the symptoms of a skin cancer in a non-human animal model.

51-53. (canceled)
